SEQUENCE LISTING (18 sequences)

SEQ ID NO: 1 - DNA, 42 bp, Artificial Sequence
  synthetic nucleic acid sequence
ctgcagcgtcagctgcagctgcagaagcctcaattgctccca

SEQ ID NO: 2 - DNA, 41 bp, Artificial Sequence
  synthetic nucleic acid sequence
ttttgggaccactgtctctccgtgggtggtgcccatggatg

SEQ ID NO: 3 - DNA, 41 bp, Artificial Sequence
  synthetic nucleic acid sequence
ttttgggaccactgtctctccgtgagtggtgcccatggatg

SEQ ID NO: 4 - DNA, 41 bp, Artificial Sequence
  synthetic nucleic acid sequence
ttttgggaccactgtctctccttgagtggtgcccatggatg

SEQ ID NO: 5 - DNA, 41 bp, Artificial Sequence
  synthetic nucleic acid sequence
cagctcactcaccactctgtcaacggctgcaacctgcggcc

SEQ ID NO: 6 - DNA, 41 bp, Artificial Sequence
  synthetic nucleic acid sequence
cagctcactcaccactctgtcaacagctgcaacctgcggcc

SEQ ID NO: 7 - DNA, 49 bp, Artificial Sequence
  synthetic nucleic acid sequence
aaagcccacaaatccaagacccattgagaagtgggtgggaaataaggta

SEQ ID NO: 8 - DNA, 66 bp, Artificial Sequence
  synthetic nucleic acid sequence
atccaagacccatatggaagagcagaagctgatctcagaggaggacctgtgagaagtgggtgggaa

SEQ ID NO: 9 - DNA, 41 bp, Artificial Sequence
  synthetic nucleic acid sequence
gcccacaaatccaagacccattgagaagtgggtgggaaata

SEQ ID NO: 10 - DNA, 77 bp, Artificial Sequence
  synthetic nucleic acid sequence
gcccacaaatccaagacccatatggaagagcagaagctgatctcagaggaggacctgtgagaagtgggtgggaaata

SEQ ID NO: 11 - DNA, 41 bp, Artificial Sequence; identical to SEQ ID NO: 9
  synthetic nucleic acid sequence

SEQ ID NO: 12 - DNA, 77 bp, Artificial Sequence; identical to SEQ ID NO: 10
  synthetic nucleic acid sequence

SEQ ID NO: 13 - DNA, 41 bp, Artificial Sequence; identical to SEQ ID NO: 9
  synthetic nucleic acid sequence

SEQ ID NO: 14 - DNA, 69 bp, Artificial Sequence
  synthetic nucleic acid sequence
gcccacaaatccaagacccatatggaagagcagaagctgatctcagaggaggacctcaggtgggaaata

SEQ ID NO: 15 - DNA, 41 bp, Artificial Sequence; identical to SEQ ID NO: 9
  synthetic nucleic acid sequence

SEQ ID NO: 16 - DNA, 42 bp, Artificial Sequence
  synthetic nucleic acid sequence
gcccacaaatccaagacccatatgggaagagcagaagcaata

SEQ ID NO: 17 - DNA, 41 bp, Artificial Sequence; identical to SEQ ID NO: 9
  synthetic nucleic acid sequence

SEQ ID NO: 18 - DNA, 53 bp, Artificial Sequence
  synthetic nucleic acid sequence
gcccacaaatccaagacccatatggaagagcagaagctgatctcagggaaata

(19) United States
(12) Patent Application Publication
(10) Pub. No.: US 2019/0218544 A1
(43) Pub. Date: Jul. 18, 2019

(54) GENE EDITING, IDENTIFYING EDITED CELLS, AND KITS FOR USE THEREIN

(71) Applicant: Takara Bio USA, Inc., Mountain View, CA (US)

(72) Inventors: Montserrat Morell Fernández, Menlo Park, CA (US); Patrick Martin, San Carlos, CA (US); Michael Haugwitz, Belmont, CA (US); Andrew Alan Farmer, Los Altos, CA (US); Baz Smith, Mountain View, CA (US)

(21) Appl. No.: 16/170,940

(22) Filed: Oct. 25, 2018

Related U.S. Application Data
(60) Provisional application No. 62/579,710, filed on Oct. 31, 2017.
(60) Provisional application No. 62/586,670, filed on Nov. 15, 2017.

(51) Int. Cl. C12N 15/10; C12Q 1/02; C12Q 1/6827; C12Q 1/6809; C12Q 1/686; C12Q 1/6876

(52) U.S. Cl. CPC C12N 15/102; C12Q 1/025; C12Q 1/6827; C12Q 2600/16; C12Q 1/686; C12Q 1/6876; C12Q 1/6809

(57) ABSTRACT

Provided are methods of identifying a cell that has been edited with a gene editing reagent. Also provided are methods of selecting a clone of a cell that has been identified as an edited cell containing a desired edit, as well as methods of gene editing that include such selecting of an edited clone. Methods of assessing the efficacy of gene editing reagents and methods employing a gene editing reagent efficacy assessment are also provided. The instant disclosure further provides kits for use in practicing the subject methods.

### CROSS-REFERENCE TO RELATED APPLICATIONS

Pursuant to 35 U.S.C. § 119(e), this application claims priority to the filing date of the United States Provisional Patent Application Ser. No. 62/579,710 filed Oct. 31, 2017 and 62/586,670 filed Nov. 15, 2017; the disclosures of which applications are herein incorporated by reference.

### INTRODUCTION

Site-specific genome editing, taking advantage of well-established as well as new powerful technologies, has found wide use in various settings. Approaches that may be employed are at various levels of development and span a wide spectrum of complexity. For example, some more complex technologies using engineered nucleases, such as such as zinc finger nucleases (ZFNs) and transcription activator like effector nucleases (TALENs), have been in development longer than some newer, less technically complex technologies, such as Clustered Regularly Interspaced Short Palindromic Repeats/CRISPR associated 9 (CRISPR/Cas9) systems. Despite the differing age of such different approaches, all of these technologies remain promising options to produce targeted edits within a wide range of cell types.

Immediately adopted by the research community, precise gene editing technologies have seen a rapid advancement in sophistication as well as adoption into applied sectors, such as agriculture and human health. For example, genome editing technologies have been effectively employed to harness the native genomic repertoire of various plants by precise alteration of DNA sequences in species as diverse as tomato, cabbage, cucumber, grape, citrus, and flowers, including chrysanthemum and lotus. Spanning public health and agriculture, gene editing has been investigated in insects to inhibit the proliferation of disease vectors and prevent insect-mediated crop destruction. With regard to human health, targeted genome editing holds great potential for treating human diseases, not only in the context of correct gene therapy, but also in strategic avenues of antiviral therapy and oncology by through targeted viral inactivation and modification of oncogene addicted cancer types, respectively.

### SUMMARY

Provided are methods of identifying a cell that has been edited with a gene editing reagent. Also provided are methods of selecting a clone of a cell that has been identified as an edited cell containing a desired edit, as well as methods of gene editing that include such selecting of an edited clone. Methods of assessing the efficacy of gene editing reagents and methods employing a gene editing reagent efficacy assessment are also provided. The instant disclosure further provides kits for use in practicing the subject methods.

### BRIEF DESCRIPTION OF THE FIGURES

FIG. 1 provides a schematic depiction of an edit consisting of a single nucleotide change.

FIG. 2 provides a schematic depiction of a junction complex sufficient for flap endonuclease-mediated cleavage that includes a desired edit at an edit site.

FIG. 3 provides a schematic depiction of a complex at an unedited target site that does not result in flap endonuclease-mediated cleavage.

FIG. 4 provides a schematic depiction of parallel assays in the case of an unedited target site and an edited target site.

FIG. 5 provides schematic depictions of deletion edits.

FIG. 6 provides a schematic depiction of an upstream insertion edit.

FIG. 7 provides a schematic depiction of one embodiment of a downstream insertion edit.

FIG. 8A-8H provide a schematic depiction of another embodiment of a downstream insertion edit as well as various flap probe and displacer oligonucleotide alignments to an insertion edit that may be employed.

FIG. 9 provides a schematic depiction of a detection strategy employing a fluorescently labeled flap probe.

FIG. 10 provides a schematic depiction of a detection strategy employing a detection cassette.

FIG. 11 provides a schematic depiction of a detection strategy employing a second flap probe, at the unedited target site, that is not cleaved by a flap endonuclease.

FIG. 12 provides a schematic depiction of a detection strategy employing a second flap probe, at the unedited target site, that is cleaved by a flap endonuclease but does not hybridize with the detection cassette.

FIG. 13 provides a schematic depiction of a detection strategy employing two different flap probes and two different detection cassettes.

FIG. 14 depicts a flap probe that includes a cleavage blocking hairpin structure.

FIG. 15 demonstrates an activity assay of an embodiment of a detection reaction using various concentrations of a FEN-1 reference enzyme.

FIG. 16 demonstrates that the herein described methods may be employed with amplified DNA templates of varying size and purity.

FIG. 17 demonstrates that the herein described methods can discriminate between the homozygous and heterozygous presence of edited and unedited alleles.

FIG. 18 demonstrates the use of the herein described methods to genotype samples and confirmation by sequencing.

FIG. 19 further demonstrates the use of the herein described methods to genotype samples and confirmation by sequencing.

FIG. 20A-20G depict the application of an embodiment of the herein described methods to detect various different individual nucleotide changes using various different amounts of starting cells and/or templates of different sizes (e.g., FIG. 20F employed a template of 700 bp in length and FIG. 20G employed a template of 300 bp in length).

FIG. 21 demonstrates that an embodiment of the herein described methods can detect low concentrations of a target edit.

FIG. 22 demonstrates the simultaneous detection of two different signals to differentiate individual nucleotide differences.

FIG. 23 demonstrates the effect on detected signal of using flap probes with different melting temperatures.

FIG. 24 demonstrates that addition of exogenous DNA to the annealing buffer in an embodiment of the herein described methods results in an increased signal-to-noise ratio.

FIG. 25 demonstrates the use of a flap probe containing a cleavage-blocking moiety to increase the signal-to-noise ratio in an embodiment of the herein described methods.

FIG. 26 demonstrates the use of dual fluorescent cassettes to detect edit heterozygosity and homozygosity in an embodiment of the herein described methods.

FIG. 27A-27B schematically depict non-limiting exemplary workflows for the detection of single nucleotide change and insertion edits.

FIG. 28A-28B demonstrate successful detection of single nucleotide edits in a heterogeneous cell population using the herein described methods as well as a comparison of the effectiveness of various different editing reagents for introducing the edit.

FIG. 29 demonstrates the successful detection of single nucleotide edits at a site within the FAH gene in individual clonal cell lines as well as the identification of homozygously and heterozygously edited cells.

FIG. 30 demonstrates the successful detection of single nucleotide edits at an alternative site with the FAH gene in individual clonal cell lines as well as the identification of homozygously and heterozygously edited cells.

FIG. 31A-31D demonstrate successful detection of large insertion edits in a heterogeneous cell population using the herein described methods as well as a comparison of the effectiveness of different editing reagents for introducing the edit.

FIG. 32 demonstrates the successful detection of large insertion edits in individual clonal cell lines as well as the identification of proper and erroneous incorporation at the 3′ and 5′ ends of the insertion using methods described herein.

FIG. 33 provides sequencing validation of the results presented in FIG. 32.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The terms “nucleic acid”, “polynucleotide” and “oligonucleotide” are used interchangeably herein to include a polymeric form of nucleotides, either ribonucleotides or deoxyribonucleotides. This term refers only to the primary structure of the molecule. Thus, the terms include triple-, double- and single-stranded DNA, as well as triple-, double- and single-stranded RNA, except where specifically designated otherwise. The terms also include such molecules with modifications, such as by methylation and/or by capping, and unmodified forms of a polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing non-nucleotidic backbones, polymers, and other synthetic sequence-specific nucleic acid polymers providing that the polymers contain nucleobases in a configuration which allows for base pairing and base stacking, such as is found in DNA and RNA. Polynucleotides also encompass those containing one or more “nucleoside analogs” or “nucleotide analogs”, which are nucleoside or nucleotide analogs of naturally occurring nucleosides and nucleotides as in e.g., RNA and DNA.

As used herein, the terms “3′ adjacent” and “5′ adjacent” with reference to nucleic acid sequences mean the nucleotide or sequence of nucleotides immediately next to the element that is referred to in the 3′ or 5′ direction, respectively. For example, a nucleotide described as 3′ adjacent to a target site, is the nucleotide immediately next to the target site in the 3′ direction. Correspondingly, a nucleotide described as 5′ adjacent to a target site, is the nucleotide immediately next to the target site in the 5′ direction. Where two nucleotides or sequences are adjacent, i.e., 3′ or 5′ adjacent, there will generally be no intervening nucleotides and/or sequence(s) between the two nucleotides or sequences, unless specifically described otherwise.

As used herein with reference to nucleotides and/or sequences of nucleotides, the terms “proximity”, “within the proximity of”, “near”, and the like, will generally refer to nucleotides and/or nucleotide sequences that are sufficiently close in physical distance so as to be capable of interacting with each other, capable of interacting with a common agent such as a common polynucleotide, and/or capable of interacting through the interaction of two or more agents that physically interact. For example, two nucleotides, two nucleotide sequences, or a nucleotide and a nucleotide sequence that are in proximity with one another may be sufficiently close such that they may hybridize to the same oligonucleotide or such that they may incorporate into the same complex of agents (including e.g., a junction complex). Nucleotides and/or nucleotide sequences in the proximity of one another may or may not be adjacent (including 3′ or 5′ adjacent as described above) and may or may not include one or more intervening nucleotides between them. In some instances, nucleotides/sequences in proximity to one another may be separated by at least 1 but not more than 100 intervening nucleotides, including e.g., at least 2, at least 3, at least 4, at least 5, at least 6, at least 7, at least 8, at least 9, at least 10, at least 15, at least 20, at least 25, at least 30, but no more than 100, 90, 80, 70, 60 or less intervening nucleotides. Two or more nucleotides/sequences that are in proximity with each other may or may not be present on the same nucleic acid strand.

The term “gene” refers to a particular unit of heredity present at a particular locus within the genetic component of an organism. A gene may be a nucleic acid sequence, e.g., a DNA or RNA sequence, present in a nucleic acid genome, a DNA or RNA genome, of an organism and, in some instances, may be present on a chromosome. Typically, a gene will be a DNA sequence that encodes for an mRNA that encodes a protein. A gene may be comprised of a single exon and no introns or multiple exons and one or more introns.

One of two or more identical or alternative forms of a gene present at a particular locus is referred to as an “allele” and, for example, a diploid organism will typically have two alleles of a particular gene. A diploid organism carrying two different alleles of a gene is said to be heterozygous for that gene, whereas a homozygote carries two copies of the same allele. A large number of genes are present in multiple allelic forms in a population. New alleles of a particular gene may be generated either naturally or artificially through natural or induced mutation and propagated through breeding or cloning. A gene or allele may be isolated from the genome of an organism and replicated and/or manipulated or a gene or allele may be modified in situ through gene therapy methods. The locus of a gene or allele may have associated regulatory elements and gene therapy, in some instances, may include modification of the regulatory elements of a gene or allele while leaving the coding sequences of the gene or allele unmodified.

Two nucleotide sequences are “complementary” to one another or have “complementarity” when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel or reverse-complement sense wherein a complementary region of a first sequence in the 5′ to 3′ orientation binds to its complementary sequence in the 3′ to 5′ orientation relative to the second and each A, T(U), G, and C of one sequence is then aligned with a T(U), A, C, and G, respectively, of the other sequence. RNA sequences can also include complementary G=U or U=G base pairs. Thus, two sequences need not have perfect homology to be “complementary”. Usually two sequences are sufficiently complementary when at least about 85% of the nucleotides share base pair organization over a defined length of the molecule, including but not limited to in some instances at least about 90%, at least about 95%, at least about 96%, at least about 97%, at least about 98%, at least about 99%, and 100% base pair organization over a defined length of the molecule.

The term “assessing” includes any form of measurement and includes determining if an element is present or not. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

As used herein, “target site” can generally refer to a site that the presence or absence of which is to be detected by the methods of the disclosure. A target site can be an unedited (i.e., wildtype) location in a gene. A target site can be an edited location in a gene (e.g., a SNP, or insertion, or deletion). A target site may arise from an editing event or any other genetic event resulting in an insertion or deletion (e.g., chromosomal translocations, deletions, recombinations). A target site may not be edited. A target site can be 1 nucleotide in length (e.g., a SNP). A target site can be located within or adjacent to an insertion or deletion.

### DETAILED DESCRIPTION

Provided are methods of identifying a cell that has been edited with a gene editing reagent. Also provided are methods of selecting a clone of a cell that has been identified as an edited cell containing a desired edit, as well as methods of gene editing that include such selecting of an edited clone. Methods of assessing the efficacy of gene editing reagents and methods employing a gene editing reagent efficacy assessment are also provided. The instant disclosure further provides kits for use in practicing the subject methods.

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present methods are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order which is logically possible.

### Methods

As summarized above, provided are methods of identifying edited cells, including cells that have been edited with one or more gene editing reagents as well as methods of editing a nucleic acid of a cell and identifying one or more cells that have been edited by the one or more gene editing reagents. Gene editing events that may be assessed and/or detected using the present methods will vary, but will generally include those where sequence upstream and downstream of a target site is known. Such editing events may include, but are not limited to, e.g., where the target site consists of a single nucleotide and sequence 3′ (i.e., 3′ adjacent) and 5′ (i.e., 5′ adjacent) of the single nucleotide target site are known. In such instances, the desired edit is also generally known, e.g., where the edit includes a nucleotide-to-nucleotide change, e.g., an A to C, A to G, A to T, C to A, C to G, C to T, G to A, G to C, G to T, T to A, T to C, T to G change, or the like. Desired edits are not limited to nucleotide changes and may include essentially any desired modification of nucleic acid sequence also including but not limited to e.g., deletions and insertions.

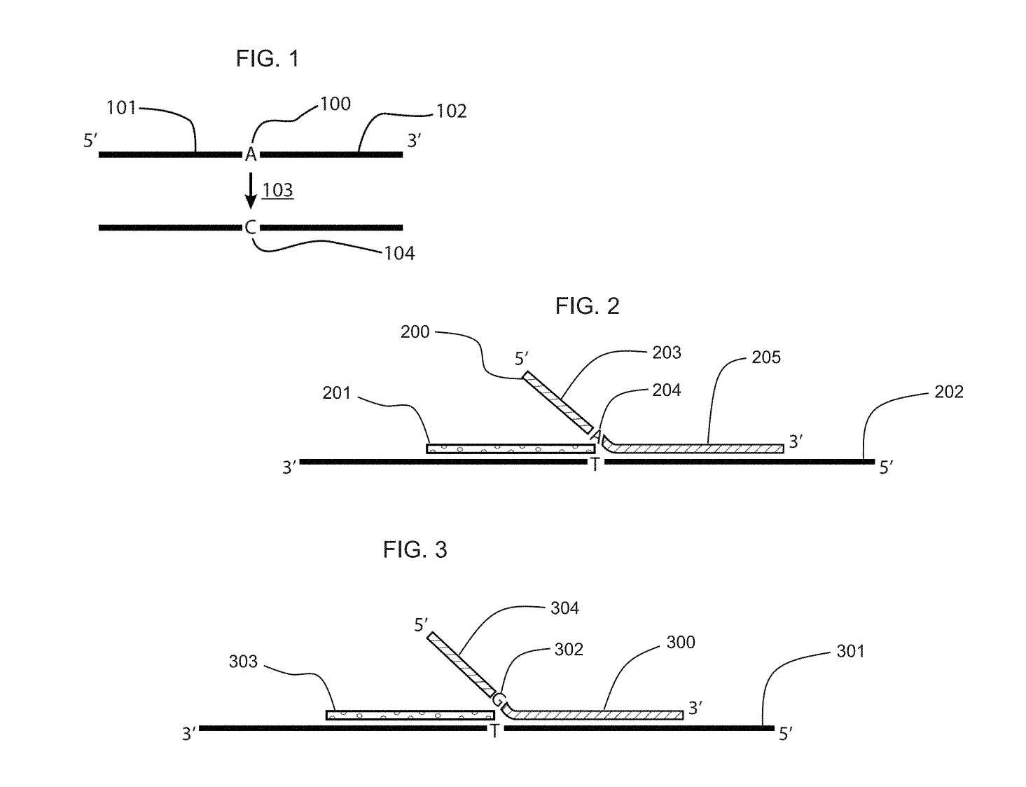

The case of a single nucleotide change at a target site is depicted in FIG. 1. Although the herein described methods may be readily applied to double-stranded target nucleic acids, for simplicity the nucleic acid in FIG. 1 is depicted as single stranded. Correspondingly, as will be readily understood, in the case of double-stranded target nucleic acids, various processes (e.g., editing, annealing, detection, etc.) may be directed to either or both strands.

Where a single nucleic acid change is desired, the unedited nucleic acid (FIG. 1, top) will include a known residue to be edited (100), an adenine (A) in the depicted case, known sequence 5′ of the target site (101) and known sequence 3′ of the target site (102). Following editing (103), the edited nucleic acid (FIG. 1, bottom) will include the desired nucleotide at the target site (104), a cytosine (C) in the depicted case, while maintaining the 5′ and 3′ known sequences adjacent to the edited residue. As described in more detail below, the edited nucleotide (104) may serve as a nucleotide of the target site to which a hinge nucleotide of a flap probe is complementary and aligned in a formed junction complex.

Accordingly, in detecting a nucleic acid that has been edited as desired at a target site the subject methods may make use of known sequence upstream and downstream of the target site to anneal oligonucleotides to the target nucleic acid after the nucleic acid has been contacted with one or more gene editing reagents. Such annealing results in the formation a particular three-stranded polynucleotide complex (i.e., a junction complex) that is recognized by an endonuclease. Recognition and cleavage of this structure by the endonuclease is subsequently detected as a means of assessing the presence and/or absence of a desired edit at the target site, thereby identifying whether the nucleic acid has been edited as desired at the target site.

A representative junction complex resulting in cleavage by an endonuclease is depicted in FIG. 2. As shown, the formed junction complex includes a flap probe (200), a displacer oligonucleotide (201) and a template nucleotide (202). The displacer oligonucleotide (201) includes a 5′ targeting region that is complementary to a region of the target nucleic acid 3′ adjacent to the target site (depicted as a single “T”). The displacer oligonucleotide will also generally include a terminal 3′ nucleotide that may or may not be configured to be complementary to a nucleotide at the target site. For example, the terminal 3′ nucleotide of the displacer oligonucleotide may be complementary to an edited nucleotide at the target site or may be arbitrary with respect to the target site. The flap probe (200) includes a 5′ flap region (203) that includes a hinge nucleotide (204) and a 3′ targeting region (205) that is complementary to a sequence of the template nucleic acid 5′ adjacent to the target site. The complementarity between the flap probe 3′ targeting region and the template nucleic acid aligns the hinge nucleotide (204) with the target site. In addition, the terminal 3′ nucleotide of the displacer oligonucleotide may contribute to the formation of the junction complex. For example, the terminal 3′ nucleotide of the displacer oligonucleotide may overlap with the flap probe at the target site, including e.g., where the terminal 3′ nucleotide is complementary with an edited nucleotide at the target site such that it overlaps with the hinge nucleotide of the flap probe in the formed junction complex.

Useful flap probes will vary depending on the target site or desired edit to which they are directed. In general, a flap probe will be a polynucleotide of greater than 15 nt in length that includes a 5′ flap region that does not substantially hybridize at or near the target site or to sequence of a desired edit, a 3′ targeting region that does hybridize at the target site and/or to at least a portion of the desired edit, and a hinge nucleotide interposed between the 5′ flap region and the 3′ targeting region which will generally be described as being within the 5′ flap region that is released upon flap endonuclease cleavage. Flap probes may range from 20 nt or less to 200 nt or more in length, including but not limited to e.g., 20 to 200 nt, 20 to 150 nt, 20 to 125 nt, 20 to 100 nt, 20 to 90 nt, 20 to 80 nt, 20 to 70 nt, 20 to 60 nt, 20 to 50 nt, 20 to 40 nt, 20 to 30 nt, 25 to 200 nt, 25 to 150 nt, 25 to 125 nt, 25 to 100 nt, 25 to 90 nt, 25 to 80 nt, 25 to 70 nt, 25 to 60 nt, 25 to 50 nt, 25 to 40 nt, 25 to 30 nt, etc. The individual region (i.e., the 5′ flap region and the 3′ targeting region) may also vary in length, including from 10 nt or less to 50 nt or more, including but not limited to e.g., 10 to 50 nt, 10 to 45 nt, 10 to 40 nt, 10 to 35 nt, 10 to 30 nt, 10 to 25 nt, 10 to 20 nt, 10 to 15 nt, 15 to 50 nt, 15 to 45 nt, 15 to 40 nt, 15 to 35 nt, 15 to 30 nt, 15 to 25 nt, 15 to 20 nt, etc. Flap probes may include one or more, or consist entirely of, naturally occurring DNA and/or RNA nucleotides. Flap probes may or may not include one or more non-naturally occurring nucleotide analogs. In some instances, a flap probe may include an extension preventing moiety, including but not limited to e.g., a 3′ spacer, such as a 3′ hexanediol spacer, a 3C phosphoramidite spacer and the like. Flap probes may or may not include attached signal producing or signal quenching moieties and/or, in certain instances, cleavage-preventing moieties, as described in more detail below related to various detection strategies.

Useful displacer oligonucleotides will vary depending on the target site or desired edit to which they are directed. In general, a displacer oligonucleotide will be a polynucleotide of greater than 15 nt in length that includes a 5′ targeting region that hybridizes at the target site and/or to at least a portion of the desired edit, and a terminal 3′ nucleotide that may or may not be complementary to a desired edit at a target site or the desired edit. In various instances, the terminal 3′ nucleotide may be arbitrary with respect to an edited nucleotide at the target site, configured to not hybridize with an edit nucleotide at the target site or arbitrary with respect to the target site besides also being configured to not hybridize with an edit nucleotide at the target site. In some instances, the terminal 3′ nucleotide may be configured to hybridize with an edit nucleotide at a target site. Displacer oligonucleotides may range from 20 nt or less to 200 nt or more in length, including but not limited to e.g., 20 to 200 nt, 20 to 150 nt, 20 to 125 nt, 20 to 100 nt, 20 to 90 nt, 20 to 80 nt, 20 to 70 nt, 20 to 60 nt, 20 to 50 nt, 20 to 40 nt, 30 to 200 nt, 30 to 150 nt, 30 to 125 nt, 30 to 100 nt, 30 to 90 nt, 30 to 80 nt, 30 to 70 nt, 30 to 60 nt, 30 to 50 nt, 30 to 40 nt, etc. Displacer oligonucleotides may include one or more, or consist entirely of, naturally occurring DNA and/or RNA nucleotides. Displacer oligonucleotides may or may not include one or more non-naturally occurring nucleotide analogs. Displacer oligonucleotides will generally not include attached signal producing or signal quenching moieties. Displacer oligonucleotides may be configured, in some instances, to prevent significant formation of secondary structure.

Elements of junction complex formation, cleavage thereof by a flap endonuclease and the subject components of junction complexes (e.g., flap probes, displacer oligonucleotides, etc.) and cleavage reactions useful in certain biotechnological applications include those described in U.S. Patent Application Pub. Nos. US20060147955A1; US20060183207A1; US20080131875A1; US2008/0187926A1; US20140057259A1; US20120231461A1; US20040203035A1; US20050048527A1; US20050186588A1; US20060147955A1; US20080131890A1; US20080131875A1; and U.S. Pat. Nos. 9,133,503; 9,096,893; 8,765,418; 5,843,669; 5,994,069; 6,090,606; 6,555,357; 6,692,917; 7,122,364; 7,381,530; 7,482,121; 7,678,541; the disclosures of which are incorporated herein by reference in their entirety.

As shown in the embodiment of FIG. 2, the hinge nucleotide (depicted as a single “A”) is complementary to the nucleotide present at the target site (i.e., the single “T” nucleotide). The complementarity between the hinge nucleotide and the target site results in a junction complex that is sufficient for cleavage by a flap endonuclease. In some instances, the terminal 3′ nucleotide of the displacer oligonucleotide may contribute to forming a junction complex sufficient for cleavage by a flap endonuclease, including e.g., where the terminal 3′ nucleotide is or is not complementary to an edited nucleotide at the target site. Such cleavage releases the 5′ flap region of the flap probe. This cleavage event may be directly or indirectly detected through various detection strategies, including e.g., those described in detail below. Such detection allows for identification of the template nucleic acid as having a desired nucleotide, e.g., a desired edited nucleotide, at the target site.

Any convenient flap endonuclease may be employed provided it has sufficient specificity for the formed junction complex. Useful flap endonucleases include those enzymes having 5′ nuclease activity that are capable of resolving three stranded junction complexes, such as the junction complexes described herein. Useful flap endonucleases include commercially available flap endonucleases, such as commercially available version of FEN-1, including wild-type and recombinantly modified versions thereof. Useful flap endonucleases include thermostable and thermolabile flap endonucleases, including e.g., thermostable FEN-1, thermolabile FEN-1, and the like. Examples of which include e.g., thermostable FEN1 available from New England Biolabs, Inc. (Ipswich, Mass.), and the like. Other useful flap endonucleases include eukaryotic flap endonucleases and prokaryotic flap endonucleases, including but not limited to e.g., bacterial flap endonucleases (e.g., E. coli flap endonucleases, (e.g., UniProt P38506, RefSeq. NP_417278.4), Salmonella flap endonucleases (e.g., UniProt P39369, RefSeq. WP_001312504.1), etc.); archaea flap endonucleases (e.g., Halobacterium flap endonucleases (e.g., UniProt Q9HQ27, RefSeq. WP_010902986.1), Thermococcus flap endonucleases (e.g., UniProt Q5JGN0, RefSeq. WP_011250232.1), Pyrobaculumand flap endonucleases (e.g., UniProt Q8ZYN2, GenBank AE009441), Archaeoglobus flap endonucleases (e.g., Archaeoglobus fulgidus FEN1, UniProt O29975, RefSeq. WP_010877775.1), etc.); yeast flap endonucleases (e.g., Saccharomyces cerevisiae (UniProt P26793, RefSeq. NP_012809.1), Schizosaccharomyces pombe (UniProt P39750, RefSeq. NP_594972.1), etc.), invertebrate flap endonucleases (e.g., fly (UniProt Q9VRJ0, RefSeq. NP_647943.2), nematode (UniProt Q9N3T2, RefSeq. NP_491168.1), etc.), plant flap endonucleases (e.g., rice (UniProt Q9SXQ6, RefSeq. XP_015639321.1), Arabidopsis (UniProt O65251, RefSeq. NP_001154740.1), etc.), and the like. Non-limiting examples of mammalian flap endonucleases include e.g., human flap endonucleases (e.g., UniProt P39748, RefSeq. NP_004102.1), mouse flap endonucleases (e.g., UniProt P39749, RefSeq. NP_001258544.1), rat flap endonucleases (e.g., UniProt Q5XIP6, RefSeq. NP_445882.1), bovine flap endonucleases (e.g., UniProt Q58DH8, RefSeq. NP_001030285.1), and the like. Such enzymes may cleave the junction complexes described herein in the context of reaction mixtures described or may be recombinantly modified to do so.

A representative complex that does not result in cleavage by a flap endonuclease is depicted in FIG. 3. As shown, complementary regions shared between the flap probe (300) and the template nucleic acid (301), align the hinge nucleotide (302), depicted as a single “G”, with the target site, depicted as a single “T”. The displacer oligonucleotide (303) may also, due to complementarity with the template nucleic acid 3′ of the target site, associate into the complex with the flap probe and the template nucleic acid. Owing to the presence of a terminal 3′ nucleotide on the displacer oligonucleotide, the displacer oligonucleotide and the flap probe may overlap at the target site. However, due to a lack of complementarity between the hinge nucleotide and the target site, the formed complex is not sufficient for cleavage by a flap endonuclease. As such, the 5′ flap region (304) of the flap probe is not cleaved and not released. Thus, the 5′ flap region of the flap probe is not detected. Such a lack of detection in the subject assay may serve to identify the template nucleic acid as not having the desired edit, e.g., a desired edited nucleotide, at the target site.

As such, in some instances, the lack of a signal from components configured to detect a desired edit may be sufficient for identifying a target site as not edited as desired. Alternatively, in some instances, the lack of a desired edit at a target site may be positively identified, including e.g., where detection of an undesired nucleotide at a target site, e.g., an unedited nucleotide or an incorrectly edited nucleotide, is based on the detection of a released 5′ flap region of a flap probe specifically configured for detection of the undesired nucleotide, the presence of an undesired edit, the presence of an unedited nucleotide, or the like. Various strategies for such detection may be employed including e.g., those described below.

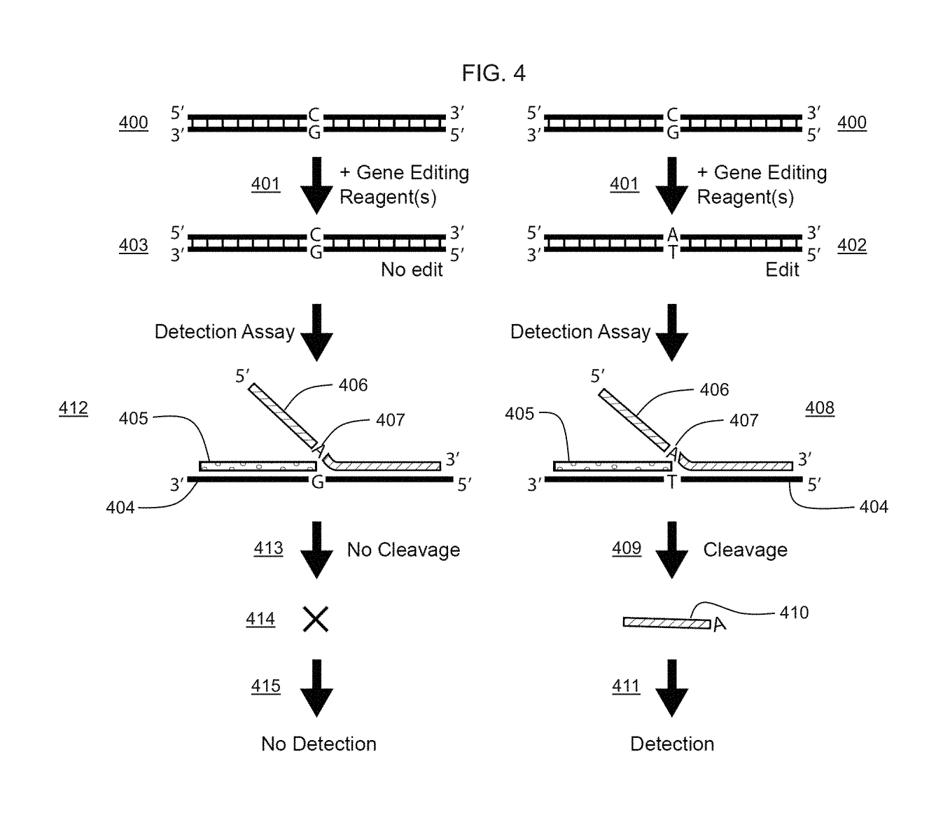

An embodiment of a method of detecting whether a target site of a template nucleic acid has a desired single nucleotide change is depicted in FIG. 4. A starting template nucleic acid (400) may be contacted (401) with one or more gene editing reagents, including e.g., one or more of the gene editing reagents described herein. Contacting with a gene editing reagent may take place in various contexts, including in vitro, in vivo, ex vivo, in ovo, etc., as described in more detail below. Following such contacting, the editing may be successful, producing the desired edit (402), or unsuccessful, producing a target site that does not contain the desired edit, e.g., an unedited target site (403) or misedited/incorrectly target site.

In the depicted example, the starting nucleic acid was contacted with gene editing reagents configured to introduce a desired G to T single nucleotide change. In order to facilitate detection of the desired change and determine whether the assayed nucleic acid contains the desired edit at the target site, a reaction mixture is prepared to perform a detection assay. The detection assay includes contacting the template nucleic acid (404), which may represent either a nucleic acid obtained directly from the cell, nucleic acid obtained from a heterogeneous population of cells, or an amplification product thereof, with a displacer oligonucleotide (405) and a flap probe (406). In this step, the displacer oligonucleotide and the flap probe are annealed to nucleic acid sequences adjacent to the target site. For example, the displacer oligonucleotide is annealed to sequence 3′ adjacent to the target site, owing to complementary sequences shared between the displacer oligonucleotide and sequence 3′ adjacent to the target site. In addition, the flap probe is annealed to sequence 5′ adjacent to the target site, owing to complementary sequences shared between the flap probe and sequence 5′ adjacent to the target site.

Such annealing aligns the hinge nucleotide (407) of the flap probe with the target site nucleotide, a single “G” at the unedited target site and a single “T” at the target site that has been edited as desired. The formed complex may further include a terminal 3′ nucleotide of the displacer oligonucleotide that is adjacent to the displacer oligonucleotide 5′ targeting region. This terminal 3′ nucleotide may be complementary to the edited “T” at the target site or may be arbitrary (i.e., any nucleotide) and may be overlapped by the hinge nucleotide of the flap probe in the formed junction complex.

Where the hinge nucleotide and the target site nucleotide are complementary, as in the nucleic acid edited as desired, the specific three-dimensional junction complex (408) is formed sufficient for cleavage of the flap probe by a flap endonuclease. In some instances, the flap endonuclease may be added (409) following the annealing step to affect cleavage, resulting in a released 5′ flap region (410) of the flap probe. Accordingly, the cleavage event releasing the 5′ flap region may be directly detected, or the reaction may be subjected to a separate detection step that indirectly detects the cleavage event (411). An example of such indirect detection includes e.g., where the released 5′ flap region is configured to act as the displacer oligonucleotide in formation of a subsequent junction complex resulting in an additional cleavage event that is detected (not depicted).

Where the hinge nucleotide and the target site nucleotide are not complementary, as in the depicted unedited nucleic acid, the specific three-dimensional junction complex does not form (412). Accordingly, upon addition of the flap endonuclease (413), no cleavage occurs and the 5′ flap region of the flap probe is not released (414). Thus, in the detection step (415), despite the presence or addition of detection components to the reaction, no signal is produced, and no detection occurs.

Alternatively, detection of an unedited site or a misedited site may be positively identified, e.g., through the use of a flap probe configured to target the unedited or misedited sequence. In some instances, e.g., where the interrogated edit is a single nucleotide change, a single displacer oligonucleotide and two or more different flap probes may be employed to positively identify both the desired edit and unedited/misedited sites.

In some instances, two or more different edited bases at the same site may be detected, including where the two or more edits are desired or where one of the two or more edited bases is desired and the others are undesired. For example, a displacer oligonucleotide and two or more different flap probes targeted to the same site, but different single nucleotide edits, may be employed. As an example, a first flap probe configured to detect an A at the relevant site and a second flap probe configured to detect a T at the relevant site may be employed, where the A or T may be both desired, both undesired, or independently desired and undesired.

While the above has been primarily described with reference to a single nucleotide change, the present methods are not so limited. Various gene editing events may be detected, provided that, following the editing event, sequence 3′ of the target site, sequence 5′ of the target site, and/or sequence inserted at the target site is known. Thus, in the case of a single nucleotide change, the changed (i.e., edited) nucleotide may serve as the target site. In the case of deletion and insertion editing events, various nucleotides may serve as the target site provided a displacer oligonucleotide and a flap probe may be designed to differentiate the edited target site from an unedited or incorrectly edited target site. In some instances, the sequence 3′ and 5′ of the target site is not known. The sequence of the target site generated by the editing event may or may not be known.

Gene editing events may include where a specific insertion or deletion is introduced at a target site, including e.g., where following the insertion or deletion, sequences 3′ and/or 5′ of the target site are known. The size of the subject insertion or deletion may vary and may range from one inserted or deleted nucleotide to an insertion or deletion of 1000 nucleotides or larger. Accordingly, insertions and/or deletions detected using the herein described methods may range from 1 nt to 1000 nt or more including but not limited to e.g., 1 nt or longer, 2 nt or longer, 3 nt or longer, 5 nt or longer, 10 nt or longer, 1 nt, 2 nt, 3 nt, 4 nt, 5 nt, 6 nt, 7 nt, 8 nt, 9 nt, 10 nt, 1 to 3 nt, 1 to 5 nt, 1 to 10 nt, 2 to 5 nt, 2 to 10 nt, 1 to 100 nt, 1 to 200 nt, 1 to 300 nt, 1 to 400 nt, 1 to 500 nt, 1 to 750 nt, 1 to 1000 nt, 5 to 10 nt, 5 to 100 nt, 5 to 200 nt, 5 to 300 nt, 5 to 400 nt, 5 to 500 nt, 5 to 750 nt, 5 to 1000 nt, 10 to 100 nt, 10 to 200 nt, 10 to 300 nt, 10 to 400 nt, 10 to 500 nt, 10 to 750 nt, 10 to 1000 nt, 50 to 100 nt, 50 to 200 nt, 50 to 300 nt, 50 to 400 nt, 50 to 500 nt, 50 to 750 nt, 50 to 1000 nt, 100 to 200 nt, 100 to 300 nt, 100 to 400 nt, 100 to 500 nt, 100 to 750 nt, 100 to 1000 nt, etc.

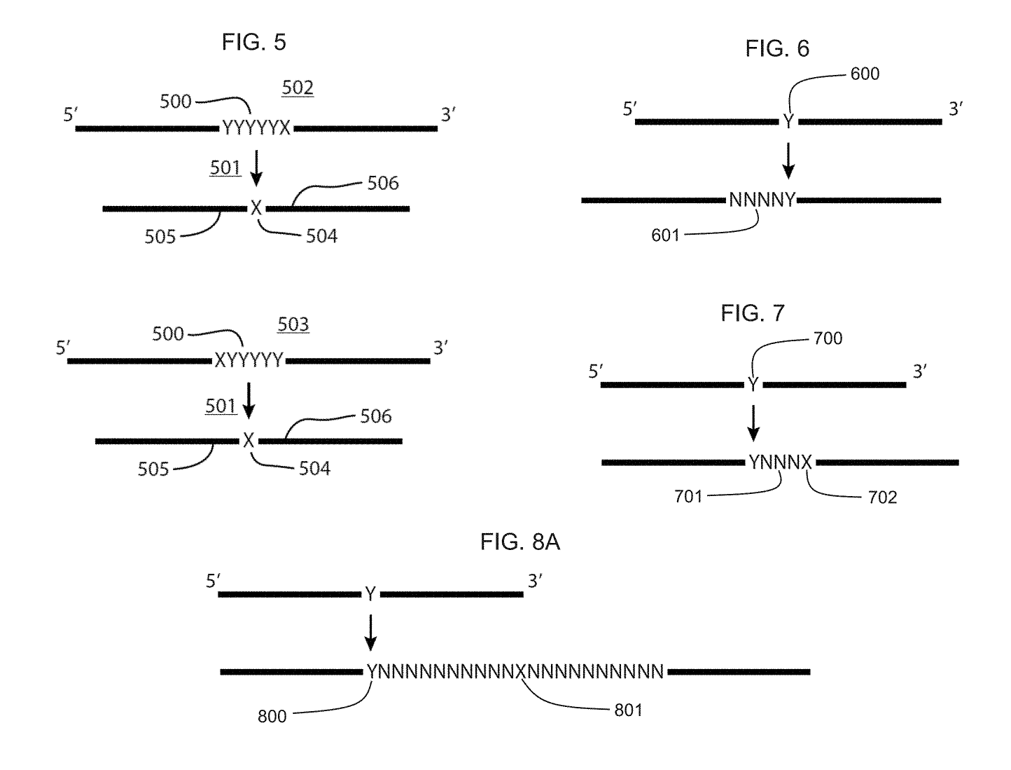

An example of a gene editing event that includes a specific deletion is depicted in FIG. 5. Specifically, an unedited template nucleic acid (500) that includes sequence to be deleted (depicted as “Y”'s) is contacted (501) with gene editing reagents configured to produce the desired deletion. The sequence to be deleted (i.e., “Y”'s) may be 5′ (502) or 3′ (503) of a nucleotide (i.e., “X”) utilized as the target site (504) to which a hinge nucleotide of a flap probe is aligned in a subsequent detection assay. Following the desired deletion event, the target site (504) that has been edited as desired includes a single nucleotide (i.e., an “X”) and known sequence 5′ adjacent (505) and 3′ adjacent (506) to the target site. Accordingly, a flap probe may be designed to have sequence complementary to the 5′ adjacent region (505) and a displacer oligonucleotide may be designed to have sequence complementary to the 3′ adjacent region (506). Thus, when the flap probe and the displacer oligonucleotide are annealed to the edited template nucleic acid, the hinge nucleotide of the flap probe is aligned with the designated target site nucleotide (504) forming a junction complex sufficient for cleavage by a flap endonuclease.

An example of a gene editing event that includes a specific insertion is depicted in FIG. 6. The target site for insertion (600) may be a single nucleotide (depicted as a “Y”) where the sequence to be inserted (601) (depicted as “N”'s) may be inserted upstream (i.e., 5′) of the target site for insertion. FIG. 7 depicts a specific insertion where the target site for insertion (700) may be a single nucleotide (depicted as a “Y”) and the sequence to be inserted (701) (depicted as “N”'s) may be inserted downstream (i.e., 3′) of the target site for insertion.

In the case of an upstream insertion (FIG. 6), the nucleotide serving as the target site for insertion (“Y”) may also serve as the target site nucleotide to which a hinge nucleotide of a flap probe is aligned in a formed junction complex. In such instances, e.g., a displacer oligonucleotide employed may have complementarity to sequence of the template nucleic acid that is 3′ of the target site for insertion and a flap probe employed may have complementarity to the sequence inserted 5′ of the target site for insertion. Such a flap probe may or may not, depending on the size of the insertion and/or the size of the flap probe, also have complementarity to sequence of the template nucleic acid that is 5′ of the end of the inserted sequence.

In the case of a downstream insertion (FIG. 7), the nucleotide serving as the target site for insertion (“Y”) may also serve as the target site nucleotide to which a hinge nucleotide of a flap probe is aligned in a formed junction complex. In such instances, e.g., a flap probe employed may have complementarity to sequence of the template nucleic acid that is 5′ of the target site for insertion and a displacer oligonucleotide employed may have complementarity to the inserted sequence (depicted as “N”'s and an “X”) that is 3′ of the target site for insertion. Such a displacer oligonucleotide may or may not, depending on the size of the insertion and/or the size of the displacer oligonucleotide, also have complementarity to sequence of the template nucleic acid that is 3′ of the end of the inserted sequence.

In another embodiment in the case of a downstream insertion (FIG. 7), a nucleotide of the inserted sequence, such as the most 3′ nucleotide (702) of the insertion (depicted as an “X”) may serve as the target site nucleotide to which a hinge nucleotide of a flap probe is aligned in a formed junction complex. In such instances, e.g., a displacer oligonucleotide employed may have complementarity to sequence of the template nucleic acid that is 3′ of most 3′ nucleotide (702) of the insertion and a flap probe employed may have complementarity to the inserted sequence (depicted as “N”'s). Such a flap probe may or may not, depending on the size of the insertion and/or the size of the flap probe, also have complementarity to sequence of the template nucleic acid that is 5′ of the end of the inserted sequence.

As will be readily appreciated, configurations of complementary sequence between an insertion-edited template nucleic acid and the flap probe and displacer oligonucleotide utilized in detection may vary and may depend on which nucleotide of the edit site is chosen for alignment with the hinge nucleotide, which may also vary. As such, in the case of upstream and downstream insertions, the nucleotide of the edited target site to which the hinge nucleotide is aligned need not necessarily be a nucleotide of the template nucleic acid adjacent to the insertion (i.e., a nucleotide immediately upstream or downstream of the insertion) or a terminal nucleotide of the inserted sequence (i.e., the 3′ or 5′ most nucleotide of the insertion). Instead the nucleotide to which the flap probe hinge nucleotide is aligned may be essentially any nucleotide within the proximity of the insertion (including nucleotides of the template nucleic acid as well as nucleotides of the inserted sequence) that allows for sufficient differentiation of an unedited target site from an edited target site using the methods described herein.

By “any nucleotide within the proximity of the insertion” is meant any nucleotide of the inserted nucleic acid sequence or any nucleotide of the template nucleic acid that may be used to align a flap probe and a displacer oligonucleotide in a manner sufficient to identify the presence or absence of the desired insertion. Identification of a desired insertion may include identifying the presence of the inserted sequence, identifying a desired 3′ junction of the inserted sequence and the template, identifying a desired 5′ junction of the inserted sequence, or a combination thereof. For example, a nucleotide may be within the proximity of the insertion when the nucleotide is any nucleotide of inserted sequence or a nucleotide of the template within one or more bases of a junction (3′ or 5′) of the inserted sequence and the template.

The distance of a nucleotide of the template from a 3′ or 5′ junction of the inserted sequence and the template may vary and may include but is not limited to e.g., the nucleotide at the junction (i.e., the nucleotide of the template adjoining the insertion), a nucleotide one or more bases from the junction, including e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more bases from the junction. The distance of a nucleotide of the template from a 3′ or 5′ insertion junction employed will not be so great; however, as to require an inappropriately long flap probe or displacer oligonucleotide, i.e., a flap probe or displacer oligonucleotide longer than would be reasonably understood from the description of such elements described herein.

By “desired junction” is generally meant that the exact intended edit at the position (3′ or 5′) where the template sequence and the inserted sequence meet is present. By contrast, “undesired junctions” may include unintended or improperly inserted sequence, including but not limited to e.g., junctions of a template sequence and a truncated insertion, junctions of a template sequence and an insertion with one or more extra spurious intervening bases, junctions of a template sequence and a truncated insertion with one or more extra spurious intervening bases, etc.

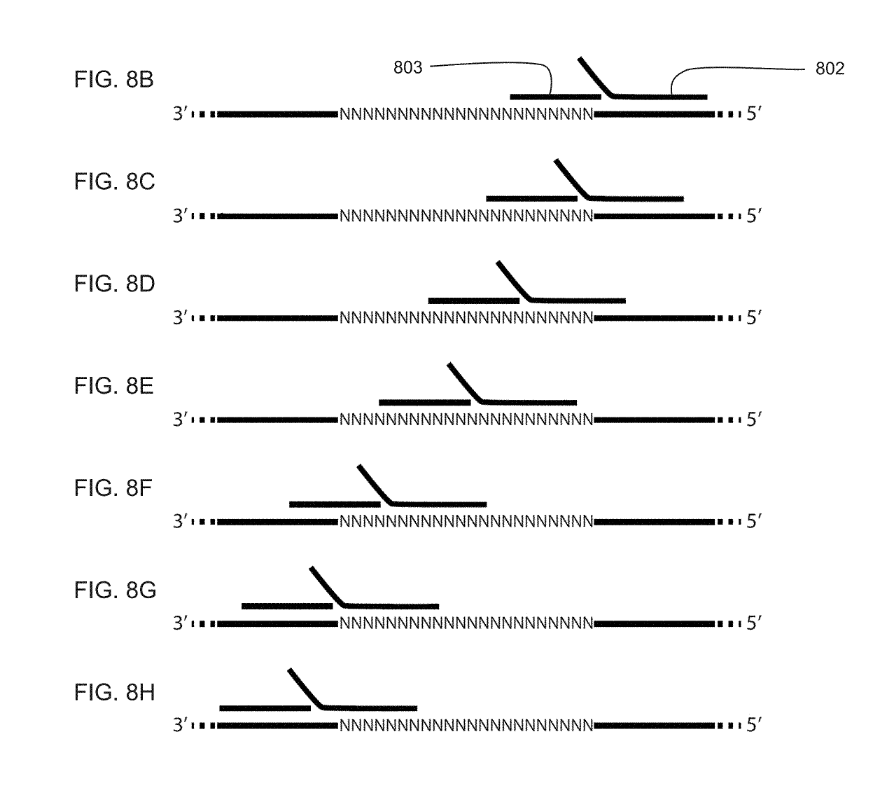

For example, as depicted in FIG. 8A, in some instances, a sequence (depicted as “N”'s and an “X”) may be inserted adjacent to a nucleotide (800) that serves as a target site for insertion (depicted as a “Y”) and a nucleotide (801) within the inserted sequence may be the nucleotide to which the flap probe hinge nucleotide is aligned. Accordingly, in some instances, both the flap probe and the displacer oligonucleotide may include sequence that is complementary to the inserted sequence. In such instances, the flap probe and/or the displacer oligonucleotide may or may not include sequence that is complementary to the template nucleic acid, including sequence of the template nucleic acid 3′ or 5′ of the inserted sequence.

FIG. 8B-8H depict various non-limiting examples of alignments of a flap probe 3′ targeting region (802) and a displacer oligonucleotide 5′ targeting region (803) with portions of a nucleic acid edited to include a desired edit of an inserted sequence (represented by N's).

As depicted in FIG. 8B, the flap probe 3′ targeting region may be complementary to a region of the nucleic acid that is 5′ of the desired edit, the displacer oligonucleotide 5′ targeting region may include a portion that is complementary to a sequence of the desired edit, and the hinge nucleotide may thus align with a nucleotide 5′ of the desired edit.

As depicted in FIG. 8C, the flap probe 3′ targeting region may be complementary to a region of the nucleic acid that is 5′ of the desired edit, the displacer oligonucleotide 5′ targeting region may be complementary to a sequence of the desired edit, and the hinge nucleotide may thus align with a nucleotide of the desired edit.

As depicted in FIG. 8D, the flap probe 3′ targeting region may include both a portion that is complementary to a sequence of the desired edit and a portion that is complementary to a region of the nucleic acid that is 5′ of the desired edit. Also, as depicted, the displacer oligonucleotide 5′ targeting region may be complementary to a sequence of the desired edit and the hinge nucleotide may thus align with a nucleotide of the desired edit.

As depicted in FIG. 8E, the flap probe 3′ targeting region may be complementary to a sequence of the desired edit, the displacer oligonucleotide 5′ targeting region may be complementary to a sequence of the desired edit and the hinge nucleotide may thus align with a nucleotide of the desired edit. In this embodiment, the flap probe 3′ targeting region and the displacer oligonucleotide 5′ targeting region are complementary to different sequences of the desired edit.

As depicted in FIG. 8F, the displacer oligonucleotide 5′ targeting region may include both a portion that is complementary to a sequence of the desired edit and a portion that is complementary to a region of the nucleic acid that is 3′ of the desired edit. Also, as depicted, the flap probe 3′ targeting region may be complementary to a sequence of the desired edit and the hinge nucleotide may thus align with a nucleotide of the desired edit.

As depicted in FIG. 8G, the displacer oligonucleotide 5′ targeting region may be complementary to a region of the nucleic acid that is 3′ of the desired edit, the flap probe 3′ targeting region may be complementary to a sequence of the desired edit and the hinge nucleotide may thus align with a nucleotide of the desired edit.

As depicted in FIG. 8H, the displacer oligonucleotide 5′ targeting region may be complementary to a region of the nucleic acid that is 3′ of the desired edit, the flap probe 3′ targeting region may include a portion that is complementary to a sequence of the desired edit, and the hinge nucleotide may thus align with a nucleotide 3′ of the desired edit.

In some instances, two or more flap probe-displacer oligonucleotide pairs may be employed, e.g., to detect the presence and/or the ends of an inserted sequence. For example, a first flap probe-displacer oligonucleotide pair may be configured to detect the properly inserted 3′ end of the insertion and a second flap probe-displacer oligonucleotide pair may be configured to detect the properly inserted 5′ end of the insertion. Essentially any convenient and appropriate combination of the alignment configurations presented in FIG. 8B-8H may be employed where multiple flap probe-displacer oligonucleotide pairs are desired.

Although the various non-limiting examples of alignments of flap probe 3′ targeting regions and displacer oligonucleotide 5′ targeting regions with portions of edited nucleic acids have been described with reference to the desired edit being an inserted sequence, one of skill in the art in view of the instant disclosure will readily recognize that in some instances the corresponding alignments and complementary portions/regions are also relevant, where appropriate, to desired single nucleotide and deletion edits as well.

The presence or absence of various edits, including nucleotide changes, insertions, deletions and combinations thereof, may be assessed using the herein described methods employing displacer oligonucleotides and flap probes configured to specific target sites and/or desired edits generated in a template nucleic acid and/or sites at, near, or within a desired edit, following the editing event. In some instances, a single flap probe-displacer oligonucleotide pair may be employed to detect or identify a desired edit. For example, in the case of a single nucleotide change, a deletion edit or an insertion edit, a single flap probe-displacer oligonucleotide pair may be employed. In some instances, multiple flap probe-displacer oligonucleotide pairs may be employed. For example, in the case of an insertion edit, multiple flap probe-displacer oligonucleotide pairs may be employed, including, e.g., where a pair is targeted to each end of the inserted sequence as described herein.

Properly configured flap probe and displacer oligonucleotide will result in formation of a junction complex at, near, or within the edit site that is sufficient for cleavage of the flap probe by a flap endonuclease. Such cleavage releases the flap probe, which may be directly or indirectly detected, as described in more detail below. Properly configured flap probe and displacer oligonucleotides will generally not result in formation of a junction complex sufficient for cleavage by a flap endonuclease in the absence of a desired edit. Any complex formed by flap probe, displacer oligonucleotide and template nucleic acid in the absence of a desired edit that the probe and oligonucleotide are configured to detect may be referred to as a complex insufficient for cleavage by a flap endonuclease.

Formation of a subject junction complex (i.e., a junction complex sufficient for cleavage by a flap endonuclease) generally involves annealing complementary regions of the components of the complex (i.e., the flap probe, the displacer oligonucleotide and the template (i.e., target) nucleic acid) to one another. The complementary regions of the components may be configured to have melting temperatures (Tm's) to allow for annealing to occur at suitable reaction conditions.

Useful Tm's may range from less than 50° C. to 75° C. or more depending on reaction conditions, including but not limited to e.g., 50° C. to 75° C., 55° C. to 75° C., 57° C. to 75° C., 60° C. to 75° C., 63° C. to 75° C., 65° C. to 75° C., 70° C. to 75° C., 72° C. to 75° C., 50° C. to 72° C., 50° C. to 70° C., 50° C. to 65° C., 50° C. to 63° C., 50° C. to 60° C., 50° C. to 57° C., 50° C. to 55° C., 60° C. to 63° C., 58° C. to 63° C., 60° C. to 65° C., 58° C. to 65° C., 70° C. to 72° C., 68° C. to 72° C., 70° C. to 74° C., 68° C. to 74° C., etc.

In some instances, a flap probe may be configured with a Tm that is different from the Tm of the displacer oligonucleotide, including where such differences range from 5° C. or more, including e.g., 10° C. or more. In some instances, a flap probe may be configured to have a Tm of 58° C. to 65° C., including but not limited to e.g., 60° C. to 63° C., 58° C. to 63° C., 60° C. to 65° C., etc. In some instances, a flap probe may be configured to have a Tm above 55° C., including e.g., above 56° C., above 57° C., above 58° C., above 58.8° C., etc. In some instances, a displacer oligonucleotide may be configured to have a Tm of 68° C. to 74° C., including but not limited to e.g., 70° C. to 72° C., 68° C. to 72° C., 70° C. to 74° C., etc.

Annealing may involve processing the reaction mixture through one or more temperatures suitable for various processes of the annealing reaction, including e.g., “melting” and/or “dissociating” the nucleic acids, “cooling”, “incubating” and the like. For example, in some instances, annealing may involve incubating the reaction mixture at a temperature at or above 85° C., including e.g., at or above 90° C., at or above 95° C. for a period of minutes (e.g., 1 min. to 10 min., e.g., 5 min.). Then the reaction may be cooled at a controlled rate (i.e., according to a specified “ramp”) to an annealing temperature. The rate of such cooling may vary and may range from less than 0.01° C./s or less to 1° C./s or more, including e.g., about 0.1° C./s. Useful annealing temperatures may be determined based on one or more Tm's of the subject components.

In some instances, an annealing reaction may be performed in the presence of exogenously added nucleic acid, including e.g., exogenously added DNA. Such exogenously added nucleic acid may, e.g., be present in or added to an annealing buffer and may be present at a concentration sufficient to increase the signal-to-noise of the assay as compared to a similar reaction performed without exogenously added nucleic acid. Such concentrations will vary and may range from less than 1 ng/μl to 100 ng/μl or more, including but not limited to e.g., 1 ng/μl to 50 ng/μl, 1 ng/μl to 10 ng/μl, 5 ng/μl to 50 ng/μl, 5 ng/μl to 10 ng/μl, etc. Such exogenously added nucleic acid may be configured to not directly interfere with hybridization of the primary annealing reaction.

Following annealing and formation of the junction complex, the flap endonuclease may be added to the reaction. Accordingly, in some instances, the flap endonuclease may not be present in the reaction mixture during the annealing reaction and, e.g., may be added following the annealing reaction in a separate step. Following addition of the flap endonuclease detection may be performed directly or indirectly, including immediately or after some period of time. Reaction components needed for the detection assay (e.g., detection cassette(s), buffers, etc.) may be added with the addition of the flap endonuclease or may be added in a subsequent and separate step. Exemplary detection strategies that may be employed are described in more detail below.

As summarized above, the instant methods may or may not include amplification of the target nucleic acid that has been contacted with one or more gene editing reagents prior to assaying for the presence of a detected edit, including e.g., prior to contacting the target nucleic acid with one or more reagents of the detection assay (e.g., a flap probe, a displacer oligonucleotide, etc.). Where employed, any convenient method of amplification may be employed, including but not limited to e.g., PCR and variants thereof. Conventional PCR generally includes the use of a pair of primers (i.e., forward and reverse), a dsDNA template and necessary reagents (e.g., dTNPs, polymerase, etc.). PCR variants useful in the subject methods include but are not limited to e.g., asymmetric PCR, nested PCR, Hot-start PCR, touchdown PCR, Assembly PCR (i.e. Polymerase Cycling Assembly), Overlap Extension PRC, Ligation-mediated PCR, RT-PCR, isothermal PCR, and the like.

Template nucleic acids that have been contacted with one or more gene editing reagents, or amplification products thereof, of various sizes may be employed in the subject methods. For example, in some instances, the starting template nucleic acid or amplification product thereof may range from less than 150 bp to more than 1000 bp in length, including but not limited to e.g., 150 bp to 1000 bp, 150 bp to 900 bp, 150 bp to 800 bp, 170 bp to 800 bp, 200 bp to 800 bp, 150 bp to 750 bp, 150 bp to 700 bp, 150 bp to 650 bp, 150 bp to 600 bp, 170 bp to 750 bp, 170 bp to 700 bp, 170 bp to 650 bp, 170 bp to 600 bp, etc.

Template nucleic acids that have been contacted with one or more gene editing reagents, or amplification products thereof, of various purities may be employed in the subject methods. For example, in some instances, the subject template nucleic acids or amplification products thereof may be purified before use in an assay of the instant disclosure. In some instances, the subject template nucleic acids or amplification products thereof may not be purified before use in an assay of the instant disclosure. For example, the subject template nucleic acids or amplification products thereof may be utilized directly in a reaction mixture prepared from lysed cells and thus containing contaminates such as cell debris, non-target nucleic acids, proteins, lipids, etc. In some instances, an amplification product is produced from the nucleic acids of a lysed cell that has been contacted with one or more gene editing reagents and the amplification product is used directly in a detection reaction of the instant disclosure, i.e., without purification of the amplification product.

### Detection Strategies

As summarized above, various detection strategies may be employed. Such strategies may directly detect a cleavage event facilitated by formation of a junction complex sufficient for flap endonuclease-mediated cleavage and presence (or addition) of the flap endonuclease in/to the reaction mixture.

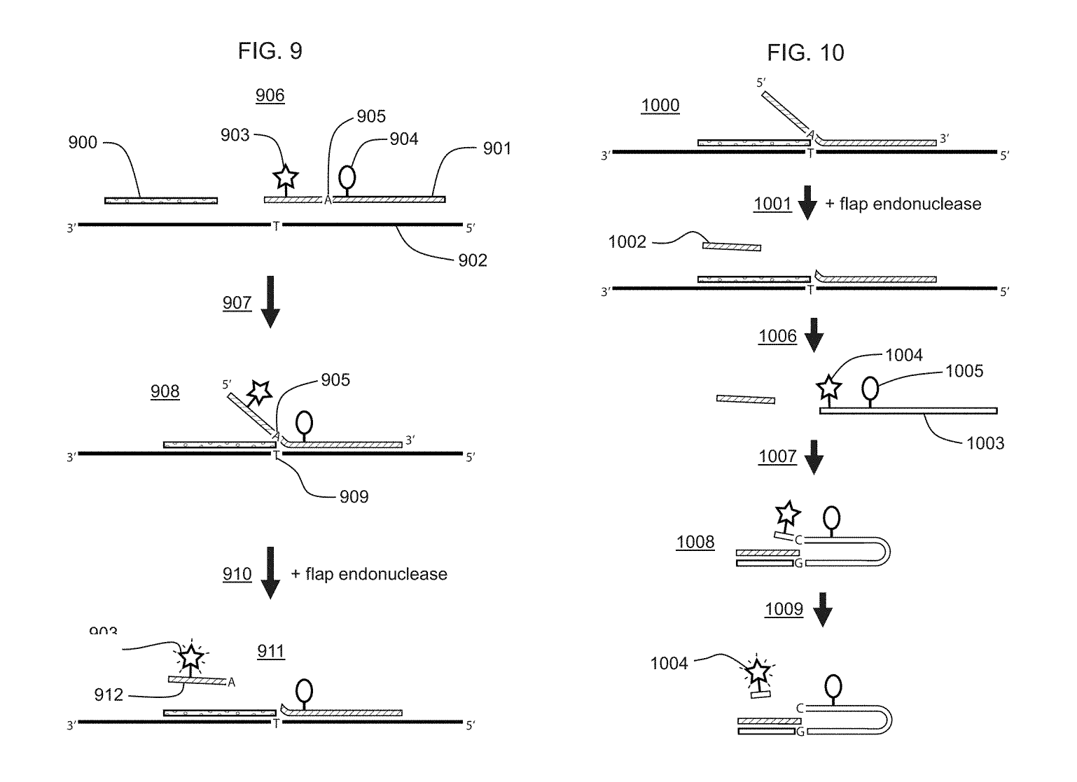

An embodiment of a direct detection strategy is provided in FIG. 9, employing a displacer oligonucleotide (900), a flap probe (901) to detect a desired edit in a template nucleic acid (902). In the subject example, the flap probe includes a fluorophore (903), the fluorescence of which is quenched by a quencher (904) also present on the flap probe at sufficient proximity to the fluorophore for quenching. The fluorophore (903) and the quencher (904) are present on opposite sides of the hinge nucleotide (905). The displacer oligonucleotide and the flap probe are contacted (906) with the template nucleic acid that has been previously contacted, e.g., in vitro or in vivo, with one or more gene editing reagents. The components are annealed (907) to form a junction complex (908) aligning the hinge nucleotide (905) with a nucleotide (909) of the template present at the target site when the template nucleic acid has been edited as desired. The annealed junction complex is contacted with a flap endonuclease (910), resulting in cleavage of the flap probe (911), releasing the 5′ flap region (912) of the flap probe thereby unquenching the fluorophore (903). In this embodiment, the cleavage event directly results in the production of a fluorescent signal, through unquenching of the fluorophore, allowing for detection of the desired edit at the target site of the template nucleic acid.

In some instances, useful detection strategies may include indirectly detecting a cleavage event facilitated by formation of a junction complex sufficient for flap endonuclease-mediated cleavage and presence (or addition) of the flap endonuclease in/to the reaction mixture. Indirect detection may involve detecting a free or released 5′ flap region of a flap probe in the reaction mixture through, e.g., utilizing the free or released 5′ flap region to generate a detectable signal. By “free” or “released” in the context of a 5′ flap region of a flap probe is meant that the 5′ flap region of the flap probe is no longer attached to another portion of the flap probe, e.g., the 3′ targeting region of the flap probe. Various approaches to detecting a free or released 5′ flap probe (including an unlabeled free or released 5′ flap probe) may be employed.

In some instances, useful detection strategies may involve a detection cassette. In this context a “detection cassette” is a nucleic acid that may both serve as a template for annealing to a released 5′ flap region of a flap probe and provide a second flap to form a second junction complex that is cleaved by a flap endonuclease.

Useful detection cassettes will include signal generation elements to produce a detectable signal. A detectable signal produced by a detection cassette will vary and may include but are not limited to e.g., fluorescent signals, colorimetric signals, luminescent signals, chromogenic signals, and the like. Useful signal generation elements that may be present on detection cassette include e.g., a fluorophore, a chromophore, a luminophore, an enzyme (e.g., an enzyme that reacts with a substrate to produce a colored reaction product), quenchers, and the like.

Useful signal generation elements include light-sensitive elements that employ Førster resonance energy transfer (FRET) to produce a signal. Such detection cassettes may be referred to as FRET-based detection cassettes and may include e.g., a fluorophore and a quencher as the subject light-sensitive elements. In comparison to a primary flap probe that is used in forming a primary junction complex, which may or may not include attached light-sensitive elements (e.g., fluorophore/quencher), a FRET-based detection cassette will generally include attached light-sensitive elements (e.g., fluorophore/quencher) to facilitate generation of a detectable signal.

An embodiment of a detection strategy employing a detection cassette is depicted in FIG. 10. A flap probe that does not include attached fluorophore/quencher is contacted with a displacer oligonucleotide and a template nucleic acid that includes a desired edit at a target site to form a junction complex (1000). The addition of flap endonuclease (1001) results in cleavage of the flap probe thereby releasing the 5′ flap region (1002) (for simplicity, here and hereafter the hinge nucleotide is not specifically depicted on the released 5′ flap region). At this point, a detection cassette (1003), which includes a fluorophore (1004) that is quenched by a quencher (1005) also present on the detection cassette at sufficient proximity to the fluorophore for quenching, is also added to the reaction mixture (1006). Alternatively, in some instances, the detection cassette may be present in the reaction mixture prior to this step. The released 5′ flap region is then annealed (1007) to the detection cassette based on complementarity shared between the released 5′ flap region and the detection cassette. A junction complex (1008) that includes the detection cassette and the 5′ flap region is formed and, through the action of a flap endonuclease, the detection cassette is cleaved (1009) thereby unquenching the fluorophore (1004) and producing a detectable signal. The flap endonuclease that cleaves the detection cassette may, in some instances, be the flap endonuclease added earlier in the assay (e.g., added for cleavage of the initial junction complex, e.g., at (1001) as depicted). In some instances, the flap endonuclease that cleaves the detection cassette may be added following annealing of the released 5′ flap region to the detection cassette (e.g., at (1009)).

In some instances, useful detection strategies may employ a second flap probe in addition to the flap probe that, together with the displacer oligonucleotide, forms the junction complex at the edited target site. In some instances, such second flap probes will not form a junction complex with the displacer oligonucleotide at the edited target site that is sufficient for flap endonuclease-mediated cleavage. However, in some instances, a second flap probe may be configured to form a junction complex at an unedited target site or a target site that includes an undesired edit or a target site with an alternative edit, such as an alternative desired edit. Second flap probes may serve various functions within a subject detection strategy, including but not limited to e.g., to increase the signal-to-noise ratio of the detection strategy, to provide a positive indication of an undesired event (e.g., an unedited target site or a misedited target site), to provide a positive indication of an alternative desired event, and the like.

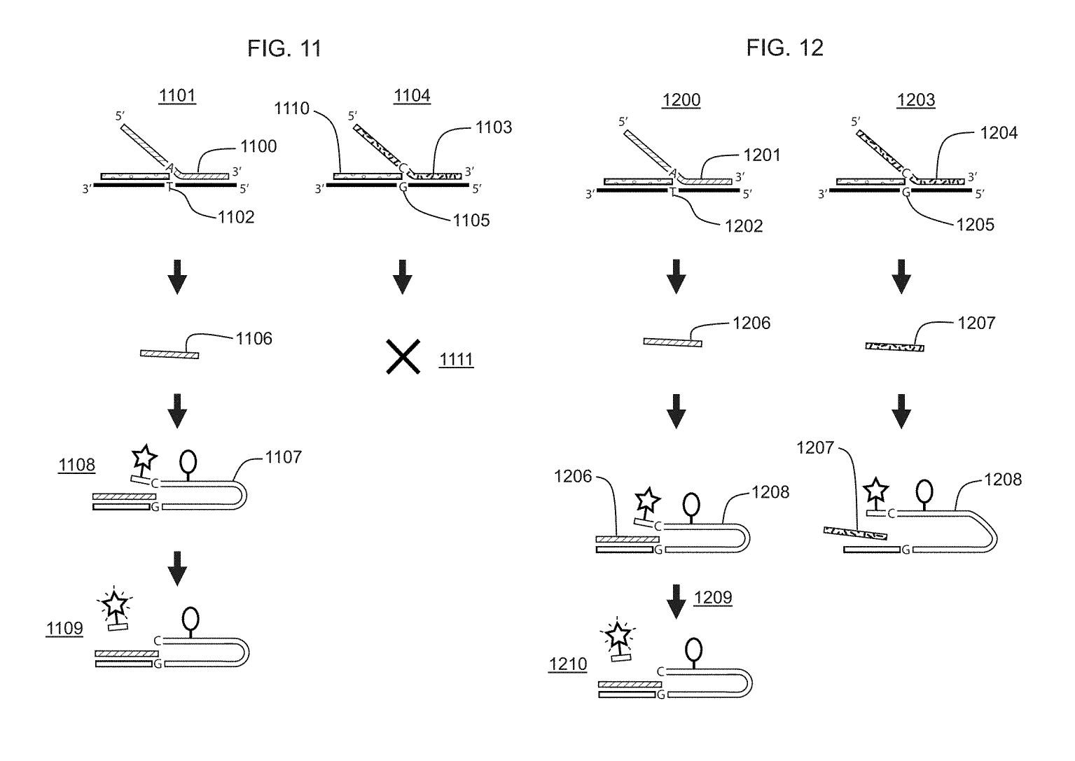

An embodiment of a detection strategy employing first flap probe (1100) configured to form a junction complex (1101) at a target site comprising a desired edit (1102) and a second flap probe (1103) configured to form a complex (1104) at an unedited target site (1105) is depicted in FIG. 11. When the junction complex (1101) is formed with the first flap probe (1100) at the target site that includes the desired edit in the presence of flap endonuclease, cleavage occurs to generate a released 5′ flap region (1106) of the flap probe. The released 5′ flap region (1106) can subsequently be detected in various ways, including e.g., through the use of a detection cassette (1107) to which the released 5′ flap region anneals (1108) and results in generation of a detectable signal (1109).

In this embodiment, the second flap probe (1103) may be configured such that annealing of the second flap probe and the displacer oligonucleotide (1110) to the unedited target site results in a complex in which the flap probe cannot be cleaved by the flap endonuclease at the unedited target site. Accordingly, no cleavage occurs at the unedited target site and the reaction does not generate free 5′ flap region of the second flap probe (1111). This lack of cleavage of the flap probe at the unedited target site may, in some instances, decrease background and increase the signal-to-noise (i.e., signal-to-background) ratio of the detection reaction.

Any convenient method of preventing cleavage of a flap probe by the flap endonuclease may be employed, including e.g., where the flap probe contains a cleavage blocking moiety. Such cleavage blocking moieties may include synthetic molecules attached to the flap probe that block cleavage by the flap endonuclease, a secondary structure of the flap probe that blocks cleavage by the flap endonuclease, and the like. Any convenient cleavage-blocking secondary structure may find use in such strategies, including e.g., self-hybridizing stem-loop or hairpin structures. An example of a flap probe containing a hairpin secondary structure that blocks cleavage by the flap endonuclease is depicted in FIG. 14. Other examples of cleavage-blocked flap probes and methods of blocking cleavage by flap endonuclease are described in Spiro et al., (1999) Molecular Cell. 4(6):1079-1085; the disclosure of which is incorporated herein by reference in its entirety. Cleavage blocking moieties may function in various ways including e.g., by blocking the binding or association of the flap endonuclease with the complex or the nucleic acid that would otherwise be cleaved, by preventing a 5′ nuclease activity required for the cleavage event, and the like.

In another embodiment, as depicted in FIG. 12, a junction complex (1200) is formed with the first flap probe (1201) at the target site that includes the desired edit (1202) and a junction complex (1203) is formed with the second flap probe (1204) at the unedited target site (1205). The formed junction complexes result in flap endonuclease-mediated cleavage and release of the first (1206) and second (1207) 5′ flap regions of the first (1201) and second (1204) flap probes, respectively. The released first 5′ flap region (1206) is configured to anneal to a detection cassette (1208), resulting in flap endonuclease-mediated cleavage (1209) of the detection cassette and generation of a detectable signal (1210). The released second 5′ flap region (1207) is configured not to anneal or hybridize to a detection cassette (1208). Thus, the released second 5′ flap region does not result in flap endonuclease-mediated cleavage of the detection cassette and no a detectable signal is generated in response to the released second 5′ flap region. This lack of flap endonuclease-mediated cleavage in response to the released second 5′ flap region may, in some instances, decrease background and increase the signal-to-noise (i.e., signal-to-background) ratio of the detection reaction.

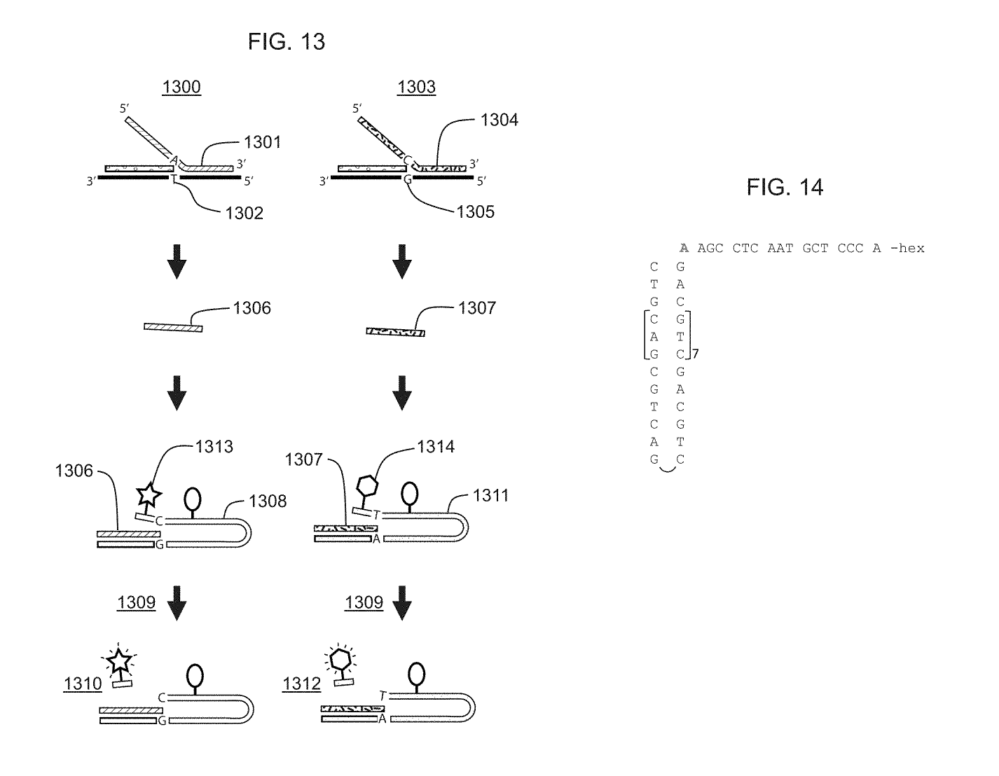

In some embodiments, a second flap probe directed to an unedited target site may be employed to positively identify the unedited target site, and or, for example, genotype a sample. For example, as depicted in FIG. 13, a junction complex (1300) is formed with the first flap probe (1301) at the target site that includes the desired edit (1302) and a junction complex (1303) is formed with the second flap probe (1304) at the unedited target site (1305). The formed junction complexes result in flap endonuclease-mediated cleavage and release of the first (1306) and second (1307) 5′ flap regions of the first (1301) and second (1304) flap probes, respectively. The released first 5′ flap region (1306) is configured to anneal to a first detection cassette (1308), resulting in flap endonuclease-mediated cleavage (1309) of the first detection cassette and generation of a first detectable signal (1310). The released second 5′ flap region (1307) is configured to anneal to a second detection cassette (1311), resulting in flap endonuclease-mediated cleavage (1309) of the second detection cassette and generation of a second detectable signal (1312). In some instances, the first (1310) and second (1312) signals produced by cleavage of the first (1308) and second (1311) detection cassettes may be of different character (e.g., different wavelengths, e.g., due to the first detection cassette having a first fluorophore (1313) that is of a different emission wavelength as compared to a second fluorophore (1314) present on the second detection cassette) or otherwise independently distinguishable.

In some instances, the use of two different signals produced that differentiate an edited target site from an unedited target site, or two differently edited target sites, allows e.g., for a comparison to be made between the relative presence of edited vs. unedited target sites, or the relative amounts of two differently edited target sites, etc. In some instances, the use of two different signals allows for quantification of the relative amount of target sites that are edited as desired as compared to the amount of target sites that are unedited, and vice versa. In some instances, the use of two different signals allows for quantification of the relative amount of target sites that are differently edited (as desired or undesired). In some instances, the use of two different signals allows for the identification of a sample or a cell as having only the desired edit, only an undesired edit, only unedited target, only an alternative desired edit, or some combination thereof. As described above, the different signals may be signals of different character (i.e., differing wavelength, differing color, different type (e.g., fluorescent vs. chromogenic), etc.).

Where a polypoid cell that has been contacted with one or more gene editing reagents is the source of nucleic acids, or amplification products thereof, assayed according to the herein described methods, dual-signal (e.g., dual-color) analysis may be employed to determine the presence or number of copies of each allele (e.g., edited allele, unedited allele, misedited allele, etc.) of interest present in the cell. For example, in the case of a diploid cell, dual-signal analysis may be employed to determine whether the cell is homozygous for a desired edit, homozygous for an unedited target or heterozygous at the subject target site. Methods may thus be employed to effectively genotype a sample. Such analysis is not limited to diploid cells and may, in some instances, find use in cells of greater ploidy, including e.g., tetraploid cells, hexaploid cells, etc.

As will be readily appreciated, in many instances employing signal generating systems that require two elements, such as FRET-based detection, the positions of the light-sensitive elements present on a subject nucleic acid may be modified and/or swapped provided the elements remain in such proximity as is necessary to generate the required interaction (e.g., a necessary FRET interaction) that is utilized to produce a detectable signal following the subject cleavage event.

A generated detectable signal may be detected by various convenient means, including e.g., using a fluorescent plate reader, an imager (e.g., a microscope), a RT-qPCR machine, and the like. As shown in the above embodiments, detected signal may be indicative of the presence of the desired edit at a target site. In some instances, a lack of signal (e.g., a lack of detected fluorescence signal) may be indicative of the absence of the desired edit at the target site. In addition, in some embodiments, detected fluorescence may be indicative of the presence of an undesired edit or a lack of an edit at a target site (i.e., “an unedited” target site) or an alternative edit at the target site. Numerous variations on these detection strategies may be implemented and the described embodiments may be readily modified for detection of particular edits, assaying of specific target sites, etc., as desired.

The described assays are not limited to the detection of an edit at a single site and may be readily employed to detect various edits at two or more sites, including were such multiplex analyses are performed simultaneously or sequentially using a single reaction mixture. The two or more sites analyzed in such approaches may be on the same or different nucleic acid strands and may be some distance of nucleotides apart, including e.g., 20 or more nt apart, 100 or more nt apart, etc. Any of the above described detection strategies, utilizing flap probe and displacer oligonucleotide targeting a first site, may be duplicated or combined with any other detection strategy, utilizing flap probe and displacer oligonucleotide targeting a second site, in order to perform two-site detection. In some instances, two-site detection, or detection of more than two sites, may employ two or more different detectable signals, e.g., to allow for differentiation of which signal corresponds with which site and the detected edit thereof.

The methods of the disclosure can be used to detect a deletion event. A deletion event may occur as a result of a gene editing event (e.g., a gene-editing event to create a knockout). A deletion can comprise any length. Deletions can comprise a few nucleotides, for example an indel comprising at least, or at most, 1, 2, 3, 4, or 5 or more nucleotides in length. A deletion can be tens, hundreds, or thousands of nucleotides in length (e.g., from 10 to 100 to 1000 to 10000 to 1000000 nt). A deletion can comprise one or more gene sequences. A deletion can comprise one or more regulatory elements controlling a gene (e.g., transcriptional and translational start sites, introns, exons, alternative splice sites, promoters, 5′ UTRs, 3′ UTRs and the like). A flap probe can be prepared to target the wildtype sequence at a pre-edited target site. If the target site is not edited, the flap probe can produce a signal according to any of the detection methods described herein. If the target site is edited and replaced with a deletion the flap probe may not produce a signal. The lack of signal can indicate that a deletion gene editing event occurred at the target site targeted by the flap probe.

The methods of the disclosure can be used to detect an insertion event. The insertion can be introduced through any means including, for example, gene-editing, homologous recombination, or chromosomal rearrangements. An insertion can comprise a sequence that was not in the original target nucleic acid. For example, an oligonucleotide can be inserted into a target nucleic acid (e.g., a gene, locus, allele, etc.). The inserted oligonucleotide can be at least or at most 10, 20, 30, 40, 50, 60, 70, 80, or 90 or more nucleotides in length. An insertion can comprise any length. Insertions can comprise a few nucleotides, for example an indel comprising at least, or at most, 1, 2, 3, 4, or 5 or more nucleotides in length. An insertion can be tens, hundreds, or thousands of nucleotides in length (e.g., from 10 to 100 to 1000 to 10000 to 1000000 nt). An insertion can comprise one or more gene sequences. An insertion can comprise one or more regulatory elements controlling a gene (e.g., transcriptional and translational start sites, introns, exons, alternative splice sites, promoters, 5′ UTRs, 3′ UTRs and the like). A flap probe and a displacer oligonucleotide can be designed to target a target site within the inserted oligonucleotide according to the methods of the disclosure (e.g., targeting the 5′ and 3′ target site adjacent regions, respectively). For example, both the flap probe and the displacer oligonucleotide can be designed to hybridize to regions within the inserted oligonucleotide. In this way, the methods of the disclosure can be used for detecting an insertion event.

In both detecting insertions and deletions the flap probes and displacer oligonucleotides can be designed to hybridize to regions adjacent to, within, or both adjacent to and within or proximal to the insertion or deletion.

### Gene Editing

As summarized above, the methods of the present disclosure generally relate to gene editing, detecting a desired gene editing event, detecting a cell edited as desired, and/or assessing the efficacy of gene editing, including e.g., assessing the efficacy of one or more gene editing reagents. Various methods of gene editing may be employed, including e.g., those methods capable of introducing a single nucleotide substitution, those methods capable of introducing a site-directed deletion and those methods capable of introducing site-directed insertion. Useful site-directed gene editing methods, described in more detail below, include methods that employ a nuclease to cleave one or both strands of a target nucleic acid molecule. The cleaved target nucleic acid may be subsequently repaired, e.g., through homology directed repair (HDR), to introduce the edit at a desired location.

Many gene editing systems, including e.g., Clustered Regularly Interspaced Short Palindromic Repeat (CRISPR)/nuclease systems, Transcription Activator-Like Effector Nuclease (TALEN) systems, Zinc Finger Nuclease (ZFN) systems, and the like, are well developed and can produce single or double strand-breaks with high fidelity. However, the efficiency of such systems for precise gene editing remains low, often requiring the screening of numerous clones in order to identify relatively few individuals containing the desired edit.

The herein described methods provide for rapid screening of nucleic acids and/or cells that have been contacted with gene editing reagents to positively identify nucleic acids and/or cells that contain a desired gene edit. In some instances, the methods of the present disclosure may be employed to detect a gene editing event in a clonal population of cells generated from a cell that has been contacted with a gene editing reagent. In some instances, the methods of the present disclosure may be employed to detect a gene editing event in a heterogeneous population of cells that has been contacted with a gene editing reagent. Heterogeneous populations of cells include naturally heterogeneous populations, such as populations collected or derived from tissue of cultures, as well as artificial heterogeneous populations, such as pooled populations of various different clonal populations and the like. The herein described methods also provide for rapid screening of gene editing reagents to positively identify those agents that effectively and/or most efficiently produce a desired edit.

Gene editing may be performed in vitro, ex vivo, in vivo, in ovo, and the like. For example, in some instances, a target nucleic acid may be contacted with one or more gene editing reagents in vitro in the absence of a host cell. In some instances, a host cell may be contacted with one or more gene editing reagents and, when present, the editing may occur in the living cell. Such host cells may be present in various forms including e.g., in a suitable vessel (e.g., a tube), in a cell culture, in an organism, etc. Cells may be collected from a subject, contacted with one or more gene editing reagents (ex vivo) and returned to the subject. Cells may be contacted with one or more gene editing reagents while present in a host organism. Cells may be contacted with one or more gene editing reagents for various purposes, including e.g., to correct a mutation present in the cell (i.e., correct a mutant allele to a wild-type allele) introduce a mutation into the cell (i.e., change a wild-type allele to a mutant allele), modify a disease-associated allele (e.g., change a disease-associated allele to an allele that is not disease-associated, introduce a disease-associated allele into a cell to study the influence of the allele on the disease, etc.), and the like

Various convenient methods of contacting a cell with one or more gene editing reagents may be employed including but not limited to e.g., transfection of gene-editing reagents or nucleic acids encoding such agents, transduction of gene-editing reagents, electroporation of gene-editing reagents, and the like.

The one or more gene editing reagents may be configured to produce a desired edit (e.g., a substitution, a deletion, an insertion, etc.) at a target site of a template nucleic acid. Useful template nucleic acids that may be edited will vary and will include cell-free (i.e., isolated) nucleic acids as well as nucleic acids present within a host cell. In some instances, a subject nucleic acid template targeted for editing will be a genomic nucleic acid of a cell, including e.g., genomically integrated nucleic acids, such as nucleic acids integrated in a prokaryotic genome, nucleic acids integrated in a eukaryotic genome and the like.

Methods of site-directed introduction of a desired edit will vary and may include introducing a site directed cleavage event, e.g., through the use of a site-directed nuclease (e.g., a CRISPR/Cas9 nuclease, a TALEN nuclease, a ZFN, and the like) followed by a specific repair event at the site cleaved by the site-directed nuclease. Such methods of specific repair may include, e.g., homologous recombination, including homology directed repair (HDR).

HDR, or similar methods, may be employed to introduce a desired mutation in a site-directed nuclease cleaved target nucleic acid, such as a genomic DNA target nucleic acid. Genomic DNA to be modified and detected according to the subject methods will vary and may include but is not limited to e.g., the genomic DNA of an animal model (e.g., used in a research setting), the genomic DNA of a plant or fungus (e.g., used in a research setting or an agricultural setting), the genomic DNA of a bacteria (e.g., used in a research setting, an agricultural setting, an industrial setting or a medical setting), the genomic DNA of an archaea (e.g., used in a research setting, an agricultural setting, an industrial setting or a medical setting),), the genomic DNA of livestock (e.g., used in a research setting, an agricultural setting, an industrial setting or a medical setting), the genomic DNA of a pet or companion animal (e.g., used in a veterinary setting), the genomic DNA of a human subject (e.g., used in a medical or clinical setting), and the like.

Animal DNA genomes that may be modified according to the methods as described herein include but are not limited to e.g., mammalian genomes including e.g., murines (rats, mice), non-human primates, humans, canines, felines, ungulates (e.g., equines, bovines, ovines, porcines, caprines), lagomorphs, etc. In some instances, a modified genome may be a plant genome, an invertebrate (e.g., a cnidarian, an echinoderm, a worm, a fly, etc.) genome, a non-mammalian vertebrate (e.g., a fish (e.g., zebrafish, puffer fish, gold fish, etc.)) genome, an amphibian (e.g., salamander, frog, etc.) genome, a reptile genome, a bird genome, a an ungulate (e.g., a goat, a pig, a sheep, a cow, etc.) genome, a rodent (e.g., a mouse, a rat, a hamster, a guinea pig) genome, etc.

Methods of homology directed repair of a DNA genome may be performed for various purposes including but not limited to e.g., the introduction of a heterologous gene or gene fragment into the genome of an organism, the correction of a deleterious mutation in the genome of an organism, the introduction of a mutation, and the like. Following the homologous recombination event the genome may be modified and thus referred to as a modified genome. Homology directed repair of a subject DNA genome may be performed in vitro, ex vivo, in utero or in vivo.

Useful site-directed nucleases, for introducing a one or more site-specific cleavage events that are repaired as desired by HDR, will vary and the selection of which may depend on e.g., the target nucleic acid to be modified (e.g., the source of the target nucleic acid to be modified and, e.g., whether human or animal target nucleic acid is to be modified), the type of modification being performed, etc. Any convenient targeting nuclease may find use in the methods as described herein.

In some instances, an instant method of editing may include the use of a Cas9 nuclease, including natural and engineered Cas9 nucleases. Useful Cas9 nucleases include but are not limited to e.g., Streptococcus pyogenes Cas9 and variants thereof, Staphylococcus aureus Cas9 and variants thereof, Actinomyces naeslundii Cas9 and variants thereof, Cas9 nucleases also include those discussed in PCT Publications Nos. WO 2013/176772 and WO2015/103153 and those reviewed in e.g., Makarova et al. (2011) Nature Reviews Microbiology 9:467-477, Makarova et al. (2011) Biology Direct 6:38, Haft et al. (2005) PLOS Computational Biology 1:e60 and Chylinski et al. (2013) RNA Biology 10:726-737, the disclosures of which are incorporated herein by reference in their entirety. In some instances, a non-Cas9 CRISPR nuclease may be employed, including but not limited to e.g., Cpf1.

Cas9 nucleases are used in the CRISPR/Cas9 system of gene editing. In the CRISPR/Cas9 system a chimeric RNA containing the target sequence (i.e., the “guide RNA” or “small guide RNA (sgRNA)”, which collectively contains a crRNA and a tracrRNA) guides the Cas9 nuclease to cleave the DNA at a specific target sequence defined by the sgRNA. The specificity, efficiency and versatility of targeting and replacement by HDR is greatly improved through the combined use of various homology-directed repair strategies and CRISPR nucleases (see e.g., Gratz et al. (2014) Genetics. 196(4)961-971; Chu et al. (2015) Nature. 33:543-548; Hisano et al. (2015) Scientific Reports 5: 8841; Farboud & Meyer (2015) Genetics, 199:959-971; Merkert & Martin (2016) Stem Cell Research 16(2):377-386; the disclosures of which are incorporated herein by reference in their entirety). The CRISPR system offers significant versatility in gene editing in part because of the small size and high frequency of necessary sequence targeting elements within host genomes. CRISPR guided Cas9 nuclease requires the presence of a protospacer adjacent motif (PAM), the sequence of which depends on the bacteria species from which the Cas9 was derived (e.g. for Streptococcus pyogenes the PAM sequence is “NGG”) but such sequences are common throughout various target nucleic acids. The PAM sequence directly downstream of the target sequence is not part of the guide RNA but is obligatory for cutting the DNA strand. Synthetic Cas9 nucleases have been generated with novel PAM recognition, further increasing the versatility of targeting, and may be used in the methods described herein.

CRISPR related gene editing components, including Cas9 nucleases and non-Cas9 nucleases, are readily available as encoding plasmids from various sources including but not limited to those available from Addgene (Cambridge, Mass.) which may be ordered online at www(dot)addgene(dot)org.

CRISPR/nuclease systems, as used herein, include Cas9 nuclease systems, systems using Cas9 variants (e.g., nickase variants, fusion variants, and the like) and systems using non-Cas9 CRISPR/nucleases and variants thereof. Nucleases of such systems include but are not limited to e.g., Class 2 type VI-A CRISPR effector C2c2 (LshC2c2), Francisella novicida Cas9 (FnCas9), Staphylococcus aureus Cas9 (SaCas9), Streptococcus thermophilus Cas9 (St1Cas9), Neisseria meningitidis Cas9 (NmCas9), Francisella novicida Cpf1 (FnCpf1), Acidaminococcus sp Cpf1 (AsCpf1), Lachnospiraceae bacterium Cpf1 (LbCpf1), SpCas9-nickase, eSpCas9, Split-SpCas9, dSpCas9-Fokl, SpCas9-cytidine deaminase, dSpCas9-gene expression functional domains, dSpCas9-Tet1 and -Dnmt3a, and the like; including e.g., those described in Murovec et al., Plant Biotechnol J. 2017 August; 15(8): 917-926; the disclosure of which is incorporated herein by reference in its entirety.

In methods of gene editing as described herein, CRISPR nucleases, including Cas9 nucleases and non-Cas9 nucleases, may be provided as a protein, including e.g., where such protein contains an attached transduction domain or is provided with one or more transduction reagents, or a combination thereof. In some instances, a CRISPR nuclease may be expressed from a plasmid or integrated into a host genome. Various methods of performing CRISPR/Cas9 mediated genome modification, including the conditions permissive for CRISPR/Cas9 mediated homology-directed repair in various settings, including in vivo and in vitro settings include but are not limited to e.g., those described in the references cited herein.

Exogenous cellular materials can be introduced to cells using any convenient protocol. Materials which may be added to cells include nucleic acids and proteins. Transfection is the introduction of foreign DNA into cells by methods which are readily available. These methods include, but are not limited to, natural competence for nucleic acid uptake in bacteria, transformation, transfection with transfection reagents including viruses (e.g., AAV or Lentivirus), electroporation, or in gesicles or micelles. Nucleic acids may be introduced into cells in single- or double-stranded form such as ssDNA fragments, dsDNA fragments, or as a plasmid. Exogenous proteins can be added to cells in any way known in the art. Proteins may be introduced into cells in a complex with a nucleic acid, such as with ribonucleoproteins, and electroporated into cells. In some instances, a CRISPR/Cas9 ribonucleoprotein complex may be electroporated into cells with along with a donor nucleic acid for homology directed repair following site-specific cleavage by the nuclease.

Proteins may be introduced into cells via transduction, protein domains for which are readily available. For example, a protein transduction domain (PTD) may be a canonical protein transduction domain (e.g., an antennapedia PTD, HIV Tat PTDs, poly-arginine PTDs, poly-lysine PTDs, and the like) or a distributed protein transduction domain (e.g., as described in U.S. Pat. No. 9,127,283; the disclosure of which is incorporated herein by reference in its entirety).

In some instances, an instant method of gene editing and detection may include the use of a zinc-finger nuclease (ZFN). ZFNs consist of the sequence-independent Fokl nuclease domain fused to zinc finger proteins (ZFPs). ZFPs can be altered to change their sequence specificity. Cleavage of targeted DNA requires binding of two ZFNs (designated left and right) to adjacent half-sites on opposite strands with correct orientation and spacing, thus forming a Fokl dimer. The requirement for dimerization increases ZFN specificity significantly. Three or four finger ZFPs target ˜9 or 12 bases per ZFN, or ˜18 or 24 bases for the ZFN pair. ZFN pairs have been used for gene targeting at specific genomic loci in insect, plant, animal and human cells. The specificity, efficiency and versatility of targeting and replacement of homologous recombination is greatly improved through the combined use of various homology-directed repair strategies and ZFNs (see e.g., Urnov et al. (2005) Nature. 435(7042):646-5; Beumer et al (2006) Genetics. 172(4):2391-2403; Meng et al (2008) Nat Biotechnol. 26(6):695-701; Perez et al. (2008) Nat Biotechnol. 26(7):808-816; Hockemeyer et al. (2009) Nat Biotechnol. 27(9):851-7; the disclosures of which are incorporated herein by reference in their entirety).

In general, one ZFN site can be found every 125-500 bp of a random genomic sequence, depending on the assembly method. Methods for identifying appropriate ZFN targeting sites include computer-mediated methods e.g., as described in e.g., Cradick et al. (2011) BMC Bioinformatics. 12:152, the disclosure of which is incorporated herein by reference in its entirety.

ZFN related components, including ZFN nucleases, are readily available as encoding plasmids from various sources including but not limited to those available from Addgene (Cambridge, Mass.) which may be ordered online at www(dot)addgene(dot)org.

In methods of gene editing and detection as described herein ZFN nucleases, may be provided as a protein, including e.g., where such protein contains an attached transduction domain or is provided with one or more transduction reagents, or a combination thereof. In some instances, a ZFN nuclease may be expressed from a plasmid or integrated into a host genome. Various methods of performing ZFN mediated genome modification, including the conditions permissive for ZFN mediated homology-directed repair in various settings, including in vivo and in vitro settings include but are not limited to e.g., those described in the references cited herein.

In some instances, an instant method of genomic DNA modification may include the use of a transcription activator-like effector nuclease (TALEN). Similar in principle to the ZFN nucleases, TALENs utilize the sequence-independent Fokl nuclease domain fused to Transcription activator-like effectors (TALEs) proteins that, unlike ZNF, individually recognize single nucleotides. TALEs generally contain a characteristic central domain of DNA-binding tandem repeats, a nuclear localization signal, and a C-terminal transcriptional activation domain. A typical repeat is 33-35 amino acids in length and contains two hypervariable amino acid residues at positions 12 and 13, known as the “repeat variable di-residue” (RVD). An RVD is able to recognize one specific DNA base pair and sequential repeats match consecutive DNA sequences. Target DNA specificity is based on the simple code of the RVDs, which thus enables prediction of target DNA sequences. Native TALEs or engineered/modified TALEs may be used in TALENs, depending on the desired targeting.

TALENs can be designed for almost any sequence stretch. Merely the presence of a thymine at each 5′ end of the DNA recognition site is required. The specificity, efficiency and versatility of targeting and replacement of homologous recombination is greatly improved through the combined use of various homology-directed repair strategies and TALENs (see e.g., Zu et al. (2013) Nature Methods. 10:329-331; Cui et al. (2015) Scientific Reports 5:10482; Liu et al. (2012) J. Genet. Genomics. 39:209-215, Bedell et al. (2012) Nature. 491:114-118, Wang et al. (2013) Nat. Biotechnol. 31:530-532; Ding et al. (2013) Cell Stem Cell. 12:238-251; Wefers et al. (2013) Proc. Natl. Acad. Sci. U.S.A, 110:3782-3787; the disclosures of which are incorporated herein by reference in their entirety).

TALEN related components, including TALEN nucleases, are readily available as encoding plasmids from various sources including but not limited to those available from Addgene (Cambridge, Mass.) which may be ordered online at www(dot)addgene(dot)org.

In methods of gene editing and detection as described herein TALEN nucleases, may be provided as a protein, including e.g., where such protein contains an attached transduction domain or is provided with one or more transduction reagents, or a combination thereof. In some instances, a TALEN nuclease may be expressed from a plasmid or integrated into a host genome. Various methods of performing TALEN mediated genome modification, including the conditions permissive for TALEN mediated homology-directed repair in various settings, including in vivo and in vitro settings include but are not limited to e.g., those described in the references cited herein.

Where a cell or a population of cells is contacted with a gene editing reagent in performing a method as described herein, the cell or cells may be sorted. Such sorting may be performed before contacting the cell or cells with the gene editing reagent(s), after the cell or cells are contacted with the gene editing reagent(s) or both before and after. For example, in some instances, cells may be collected (e.g., from a subject) and may be sorted to select a particular cell or a particular cell type with which to contact with the one or more gene editing reagents.

In some instances, cells contacted with the one or more gene editing reagents may be sorted, e.g., to isolate individual cells for various purposed, including cloning and/or use in a detection assay as described herein. As an example, a plurality of cells may be contacted with one or more gene editing reagents and the cells may be sorted into individual vessels of a multi-vessel device (e.g., individual wells of a multi-well device). Sorting may be performed according to any convenient method including e.g., where the target cells are sorted cytometrically. Cytometric sorting of the present methods will vary and may include but is not limited to e.g., sorting performed using a flow cytometer, sorting performed using a cell cytometer, sorting performed using a microfluidics cell sorter, and the like.

In some instances, cell sorting may employ a protocol to isolate individual cells, including but not limited to e.g., a limiting dilution protocol. Limiting dilution sorting may or may not be employed and, where employed, may be used at various points in a particular procedure. For example, in some instances, cells contacted with one or more gene editing reagents are sorted according to a limiting dilution protocol to isolate individual cells, e.g., to be individually cloned, to be individually screened for the presence of a desired edit, etc.

Once in individual vessels, the cells may, e.g., be cloned to generate clones of each individual cell. The generated clones may be used in various downstream procedures. For example, in some instances a clone of the cell may be retained and a separate clone of the same cell may be subjected to a detection assay, as described herein, to detect whether cell, and thus the corresponding retained clone, contains the desired edit. Where multi-vessel devices are employed, a subject procedure, e.g., a cloning procedure, a detection assay, etc., may be performed on all vessels of the device or a portion of the vessels of the device. For example, in some instances, an entire multi-vessel device (i.e., cells in each vessel of the device) may be screened for the presence/absence of a desired gene edit and, where the desired edit is detected, the corresponding clone(s) may be retrieved and employed in various downstream uses, including e.g., those described herein.

Produced clonal populations may be screened individually or they may be pooled into a heterogenous population for screening. Pooling may, in some instances, allow for rapid identification of the presence or absence of a desired edit within a population of clones that may or may not, as desired, be further screened individually. As described herein, the instant methods are applicable to screening and detection in both clonal and heterogeneous cell populations. In some instances, screening of heterogenous populations may allow for the detection of multiple different editing events in the population (e.g., an insertion event and a single nucleotide change). Multiplex screening of heterogeneous populations may, in some instances, employ multiple flap probe-displacer oligonucleotide pairs, including where such pairs are each directed to a specific edit, as desired.

Following the production of an edited nucleic acid and/or contacting a target nucleic acid with a gene editing reagent, as described herein, the edited and/or contacted nucleic acid may be purified or washed prior to one or more subsequent steps. Nucleic acids may be prepared (including by lysing cells, with or without purification) from clonal populations or heterogeneous cell populations as desired. Useful methods of purifying and/or washing generated nucleic acid molecules include but are not limited to e.g., column-based dsDNA purification (e.g., DNA purification columns), nucleic acid extraction methods (e.g., alcohol extraction), gel-based purification methods, and the like.

In some instances, following the production of an edited nucleic acid and/or contacting a target nucleic acid with a gene editing reagent, as described herein, the edited and/or contacted nucleic acid may not be purified or washed prior to one or more subsequent steps. For example, in some instances, a nucleic acid may be contacted with a gene editing reagent and, following such contacting, the nucleic acid may be immediately employed (with or without intervening amplification) in a detection assay as described herein. In some instances, a cell may be contacted with a gene editing reagent and, following such contacting, the cell may be lysed and immediately employed (with or without intervening amplification) in a detection assay as described herein.

Cells and/or nucleic acids contacted with one or more gene editing reagents, including reagents of CRISPR/nuclease systems, TALEN systems and ZFN systems, may be edited at a low frequency. Thus, where a population of cells is contacted with such reagents, only a relatively small number of cells may contain the desired edit. Accordingly, the instant methods may be employed to identify such cells. By “low frequency” and “relatively small” in this context is meant that the desired event happens in some instances at a frequency of less than 10%, including less than 5%, and including less than 1%, and thus the cells having the desired event may represent less than 10%, including less than 5%, and including less than 1%, of the overall population.

### Assessing Editing Reagent Efficacy

As summarized above, also provided are methods of assessing the efficacy of one or more gene editing reagents. Such methods may find use, e.g., in screening a population of gene editing reagents for the ability to produce a desired edit at a frequency greater than other reagents of the population. Reagents identified as efficacious gene editing reagents may be employed in downstream gene editing. Such an approach may result in the final gene editing process being more efficient that it would have been without the subject assessment and selection of efficacious gene editing reagent(s).

Method of assessing the efficacy of gene editing reagents may involve contacting a population of nucleic acids or cells with a population of different gene editing reagents and detecting cells that have been edited as desired by the reagents. In some instances, the relative frequency of produced desired edits may be quantified for each reagent of the population or a portion thereof. Methods that may be employed for detecting cells that have been edited as desired by the reagents include those of the above described methods. Specifically, any detection method employing a flap endonuclease-mediated cleavage and/or any of the detection strategies described above may be employed in the instant methods of assessing the efficacy of one or more gene editing reagents.

Such methods of assessing the efficacy of one or more gene editing reagents, as well as the method of detecting editing cells described above, may be non-diagnostic methods. As the methods may be “non-diagnostic methods,” they may be methods that do not determine a disease (e.g., sickness) or condition (e.g., a genetic predisposition) in a living organism, such as a mammal, e.g., a human. As such, methods of the instant disclosure may not, in some instances, be methods that are employed to determine whether a living subject has a given disease or condition.

Methods for assessing the efficacy of one or more gene editing reagents may determine whether a target nucleic acid comprises a desired edit at the target site based on detection of a released 5′ flap region to assess the efficacy of the gene editing reagent with which the nucleic acid, or a cell containing the nucleic acid, or an amplification product thereof, was contacted.

In some instances, methods assessing the efficacy of one or more gene editing reagents may involve the detection of a detectable signal produced in response to release of a 5′ flap region of a flap probe indicating an assayed condition, e.g., the presence or absence of the desired edit. Such methods may employ direct detection of cleavage by flap endonuclease or indirect detection, e.g., through the use of one or more detection cassettes. In some instances, multiple different signals may be employed e.g., to allow for quantification of the relative presence of desired edits versus unedited target sites in a particular sample or population.

In some instances, the subject methods may assess the efficacy of a plurality of gene editing reagents each combined into an individual reaction mixture present in an individual reaction vessel. Any convenient vessel, e.g., tubes, dishes, culture flasks, etc., may be employed. The plurality of gene editing reagents may all target the same edit site or two or more different target sites may be targeted. For example, the plurality of gene editing reagents may all target the same edit site and the plurality of gene editing reagents may all be configured to introduce the same edit (e.g., replace the same nucleotide, introduce the same deletion, introduce the same insertion, etc. In such instances, the efficacy of the different gene editing reagents may be directly compared to identify which reagents are efficacious and/or which reagent(s) are the most efficacious. Any corresponding population of different gene editing reagents may be evaluated for relative efficacy in producing a desired edit including e.g., where the plurality of gene editing reagents comprise a plurality of different gRNAs, a plurality of different TAL effector DNA-binding domains, a plurality of different ZFNs, combinations thereof and the like.

In some instances, assessments of gene editing reagent efficacy may be performed in multiplex. Including e.g., where individual reaction vessels of a multi-vessel device are employed (e.g., a multi-well plate). According the reagents (i.e., gene editing reagents, detection assay reagents, cells, etc.) may be arrayed for side-by-side assessment in multiplex fashion and/or subsequently processed through method steps by transfer of all or a portion of the contents of the individual vessels into a subsequent multi-vessel device.

Assessments of the efficacy of gene editing reagents using methods that detect the presence of desired edits, as described herein, may further include one or more control reactions or reference standards. Useful controls include positive controls (e.g., cells or nucleic acids known to contain the desired edit, gene editing reagents that are known to be efficacious or provide a reference level of operability, etc.). In some instances, positive controls may confirm the presence of an edited nucleotide at an edit site of a control target nucleic acid. Useful positive controls may also include essentially any nucleic acid with an edit, including a complete or partial insertion edit, a complete or partial deletion edit, and the like. Useful controls include negative controls (e.g., cells or nucleic acids known not to contain the desired edit, gene editing reagents that are known to not be efficacious or provide a reference level of inoperability, etc.). In some instances, negative controls may confirm the absence of the edited nucleotide. Useful negative controls may also include essentially any nucleic acid with a partial or absent edit, including a partial or absent insertion edit, a partial or absent deletion edit, and the like. Experimental reagents (i.e., gene editing reagents for which efficacy is unknown) may, in some instances, be evaluated in comparison to controls. Experimental reagents may, in some instances, be evaluated internally, e.g., by comparison relative to other experimental reagents.

Once efficacious gene editing reagents are identified, through the methods described herein, such agents may be employed in editing cells to produce the desired edits. Such edited cells may or may not be further screened for the presence/absence of the desired edit(s), e.g., through the use of methods described above. Cells edited as desired, and positively identified as such, may find use in a wide variety of applications, described in more detail below.

### Uses of Edited Cells

As summarized above, in some instances, the methods described may be employed to produce an edited cell having a desired edit, where such edits may include one or more single nucleotide changes, an insertion, a deletion or some combination thereof. In some instances, the instant methods may include preparing a clone of a cell that has been contacted with a gene editing reagent and maintaining the clone while a determination as to whether the desired gene edit has actually been produced in the cell. Such clones may be maintained in any convenient manner, including e.g., continued culture, cryopreservation, developing or incorporating the clone into a living multicellular organism, and the like.

In some instances, the subject methods may provide for rapid screening of cells that have been contacted with one or more gene editing reagents to identify one or more clones of the cells that contain a desired gene edit. Such screening may be performed on a plurality of cells that have been contacted with a gene editing reagent. Clones of the cells undergoing screening may be maintained in such a manner that the cellular source of each clone is retained. Any convenient method of tracking the source of each clone may be employed, including but not limited to e.g., labeling a vessel containing each clone with an identifier that corresponds to the cellular source that is subsequently or concurrently assayed to determine if it contains the desired gene edit. In instances where the plurality of cells contacted with the gene editing reagent(s) are sorted into individual vessels of a multi-vessel device (e.g., a multi-well plate), clones of the cell in each vessel may be arrayed into a corresponding multi-vessel device, e.g., with a similar arrangement. This approach may, for example, allow for the identification of a clone corresponding with a particular source cell by identifying the vessel at a position, e.g., column and row, in a multi-vessel device containing clones that corresponds with the position, e.g., column and row, in a multi-vessel device containing the source cells of the particular cell. In some instances, multiple individual containers, e.g., tubes, culture flasks, etc., may be employed rather than a multi-vessel device. Various other methods of retaining the relationship between source cells and clones will be readily apparent.

Once a clone is identified as containing a desired edit, the clone may be retrieved and utilized in various downstream applications. Retrieval of the clone may vary depending on how the clone is maintained, and may include but is not limited to e.g., obtaining an aliquot from a cell culture of the clone, thawing a cryopreserved clone, obtaining a cellular sample from a multicellular organism produced from the clone, obtaining a cellular sample from a multicellular organism into which the clone has been incorporated, etc. In some instances, the clone may be further confirmed as containing the desired edit prior to, during or after further downstream applications. Various methods may be employed for confirming the presence of the desired edit, including but not limited to e.g., sequencing nucleic acid from the clone that has been identified as containing the edit according to the methods described herein, including where any convenient and appropriate sequencing method is employed, such as e.g., Sanger sequencing or other high fidelity sequencing method, such as e.g., SMRT sequencing and the like. In some instances, confirmation that the clone contains the desired edit may not be performed and/or may not be necessary.

As summarized above, various downstream applications may employ edited cells identified according to the methods described herein, such methods may include but are not limited to e.g., environmental applications, agricultural applications, drug screening applications, direct medical applications (e.g., corrective mutation applications), and the like.

In some instances, cells identified as edited cells may be employed in developing CRISPR/Cas9, TALEN or ZFN homology-directed repair modified animals, including mammals, e.g., non-human mammals. Such systems may be employed to produce mammals with modified genomes. For example, mammal embryos (e.g., bovine embryos, porcine embryos, mouse embryos, and human embryos) may be contacted with one or more gene editing reagents, and contacted mammal embryos that have been edited as desired may be screened and identified according to the methods described herein.

From mammalian embryos identified as having a desired edit, mammals with the correctly modified genomes may be produced. Non-limiting examples of mammals with genomes modified as desired include horn-less cows, goats that produce more cashmere, mouse models that overcome hereditary deafness, mouse models that overcome Huntington's disease, and monkey model system embryos with disrupted dystrophin genes and the like. In some instances, human mammalian cells may be of interest, including e.g., stems cells of cystic fibrosis patients with corrected genomes. Non-limiting examples of mammals, mammalian research models, modified mammalian cell models, etc., with genomes modified as desired include e.g., those described in Carlson et al (Nature 2016) Production of hornless dairy cattle from genome-edited cell lines; Wang et al (PLOS 2016) Disruption of FGF5 in Cashmere Goats Using CRISPR/Cas9 Results in More Secondary Hair Follicles and Longer Fibers; Regalado, Antonio (MIT Technology Review 2017) “The Easiest Place to Use CRISPR Might Be in Your Ear”; Schwank, Gerald et al. (Cell Stem Cell 2013) “Functional Repair of CFTR by CRISPR/Cas9 in Intestinal Stem Cell Organoids of Cystic Fibrosis Patients”; Yang, Su et al (JCI 2017) “CRISPR/Cas-9-mediated gene editing ameliorates neurotoxicity in mouse model of Huntington's disease”; Chen, Yongchang (Human Molecular Genetics 2015) “Functional disruption of the dystrophin gene in rhesus monkey using CRISPR/Cas9”; the disclosures of which are incorporated herein by reference in their entirety.

In some instances, cells identified as edited cells may be employed in developing methods to improve the safety of xenotransplantation. Such improvements may be employed in organ transplants. For example, mammalian cells may be contacted with one or more gene editing reagents and contacted mammal cells that have been edited as desired may be screened and identified according to the methods described herein. From the mammal cells identified as having the desired edit, mammalian cells containing genomes modified as desired and/or post-embryonic mammals containing the desired edit may be produced. Non-limiting examples of mammal cells, which may be used to produce post-embryonic mammals, containing modified genomes include those having inactivation of PERV genes used to generate improved pig organs for xenotransplantation, including e.g., those described in Niu, Dong et al (Science 2017) “Inactiation of porcine endogenous retrovirus in pigs using CRISPR-Cas9”; Feng, Wanyou et al (International Journal of Molecular Sciences 2015). “The Potential of Combination of CRISPR/Cas9 and Pluripotent Stem Cells to Provide Human Organs from Chimaeric Pigs”; Servick, Kelly (Science Mag 2017) “CRISPR slices virus genes out of pigs, but will it make organ transplants to humans safer?”; PG Pub No. US20170251646A1; the disclosures of which are incorporated herein by reference in their entirety.

As another example, cells identified as edited cells may be employed in developing RNA-guided CRISPR gene drive systems. Such systems may be employed in modified organisms including those that influence human health as disease vectors (e.g. mosquitos). In some instances, gene drive systems may be employed for the reduction or elimination of a species. For example, mosquito embryos may be contacted with one or more gene editing reagents and the contacted mosquito embryos that have been edited as desired, i.e., to incorporate a subject gene drive system, may be screened according to the methods described herein. From the mosquito embryos identified as having the desired edit, mosquitos containing components of a gene drive system may be produced. Non-limiting examples of organisms that may be modified to contain components of a gene drive system include but are not limited to e.g., RNA-guided CRISPR gene drive containing mosquitos, fruit flies, and RNA-guided CRISPR gene drive containing flour beetles, including e.g., those described in Hammond et al (Nature Biotechnology 2015), “A CRISPR-Cas9 gene drive system targeting female reproduction in the malaria mosquito vector Anopheles gambiae”; PG Pub No. US20160333376A1; Gantz, Valentino et al (Science 2015) “The mutagenic chain reaction: A method for converting heterozygous to homozygous mutations”; Drury, Douglas et al (Science 2015) “CRISPR/Cas9 gene drives in genetically variable and nonrandomly mating wild populations”; the disclosures of which are incorporated herein by reference in their entirety.

In some instances, cells identified as edited cells may be employed in developing RNA-guided CRISPR, TALEN or ZFN genome edited plants. Such plants include altered agriculture genomes having certain beneficial characteristics for agricultural practices. For example, plant cells (e.g., tomato cells) may be contacted with one or more gene editing reagents and the contacted plant cells that have been edited as desired may be screened and identified according to the methods described herein. From the plant cells identified as having the desired edit, plants with the desired genome edits may be produced. Non-limiting examples of plant cells with useful genome edits include, tomatoes with better yield, wheat safe for those with Celiac disease, herbicide-resistant rice plants, canker-resistant citrus, and drought-resistant plants including e.g., those described in U.S. Pat. No. 9,750,214; Shimatani, Zenpei et al (Nature Biotechnology 2017) “Targeted base editing in rice and tomato using a CRISPR-Cas9 cytidine deaminase fusion; Peng A et al (Plant Biotechnology 2017) “Engineering canker-resistant plants through CRISPR/Cas9-targeted editing of the susceptibility gene CsLOB1 promoter in citrus”; the disclosures of which are incorporated herein by reference in their entirety.

In some instances, cells identified as edited cells may be employed in developing RNA-guided CRISPR, TALEN or ZFN genome edited fungi, including e.g., agriculturally relevant fungi. Such fungi may include altered genomes having certain beneficial characteristics for agricultural practices. For example, fungi cells (e.g., mushroom cells) may be contacted with one or more gene editing reagents and the contacted fungi cells that have been edited as desired may be screened and identified according to the methods described herein. From the fungal cells identified as having the desired edit fungi with the desired genome edits may be produced. Non-limiting examples of fungal cells with useful genome edits include non-browning mushrooms, and edited filamentous fungi, including e.g., those described in Waltz (Nature 2016) “Gene-edited CRISPR mushroom escapes US regulation”; PG Pub Nos. 20170114361; Nødvig, Christina et al (PLOS One 2015) “A CRISPR-Cas9 System for Genetic Engineering of Filamentous Fungi”; the disclosures of which are incorporated herein by reference in their entirety.

In some instances, cells identified as edited cells may be employed in developing RNA-guided CRISPR, TALEN or ZFN genome editing to treat disease. Such systems may be employed in corrective mutations. For example, human cells (e.g., human stem cells, human progenitor cells, human somatic cells, etc.) may be contacted with one or more gene editing reagents and the contacted human cells that have been edited as desired may be screened and identified according to the methods described herein. From the human cells identified as having the desired edit additional humans cells and/or tissues/organs containing the corrective mutations may be produced. Non-limiting examples of human cells containing corrective mutations include cells that are resistant to HIV/AIDS, cells that are resistant to specific allergies, cells corrected for sickle cell anemia, and cells for treating cancer, and the like, including e.g., those described in U.S. Pat. No. 9,757,420; PG Pub Nos. UA20170183413A1; Paul, John et al (Nature Methods 2017), “Combinatorial CRISPR-Cas9 screens for de novo mapping of genetic interactions”; Daesik et al (Nature Biotechnology 2017) “Genome-wide target specificities of CRISPR RNA-guided programmable deaminases”; PG Pub No. US20170021011A1; the disclosures of which are incorporated herein by reference in their entirety.

In some instances, cells identified as edited cells may be employed in genome-scale drug screening assays. Such assays may be employed in identifying particular interactions between certain drugs or drug classes and individual genomic factors, e.g., factors that may be present in an individual subject or a population of subjects treated with the drug(s). For example, cells (e.g., mammalian cells, including e.g., cultured or primary mouse cells, cultured or primary human cells, and the like) may be contacted with one or more gene editing reagents and the contacted cells that have been edited as desired may be identified according to the methods described herein. The cells identified as having the desired edit may then be subjected to screening with an array of compounds, e.g., to test the influence of a particular genomic factor on drug response. Alternatively, an array of different gene edits in a plurality of populations of cells may be produced and identified, according to the methods described herein, and each population of the plurality may be screened with an individual drug or a class of drugs. Such approaches may serve to identify the influence of a particular drug or class of drugs across various genomic factors. A non-limiting example of CRISPR-based screening includes genome-wide loss-of-function screening, including but not limited to e.g., that described in Shalem O (Science 2014) “Genome-scale CRISPR-Cas9 knockout screening in human cells”; the disclosure of which is incorporated herein by reference in its entirety.

### Kits

Also provided are kits having one or more components and/or reagents and/or devices, where applicable, for practicing one or more of the above-described methods. The subject kits may vary greatly. Kits of interest include those having one or more reagents mentioned herein, and associated devices where applicable, with respect to the methods of gene editing, determining whether a cell that has been contacted with a gene editing reagent includes a desired gene edit, cloning an identified edited cell, using an identified edited cell, assessing the efficacy of a gene editing reagent, and the like.

Kits for use in methods of the present disclosure may include a flap endonuclease. Flap endonucleases include those enzymes having 5′ nuclease activity that are capable of resolving three stranded junction complexes, as described herein. Flap endonucleases are members of the 5′ nuclease superfamily, whose members perform a wide variety of roles in nucleic acid metabolism using a similar nuclease core domain that displays common biochemical properties and structural features. A detailed review of flap endonuclease structure and function is provided in Finger et al. (2012) Subcell Biochem. 62:301-326; the disclosure of which is incorporated herein by reference in its entirety. Various crystal structures of flap endonucleases have been solved (see e.g., the structures of Protein Data Bank (PDB) ID Nos.: 1A76, 1A77, 1MC8, 1U76, 1U7B, 1UL1, 1 UTS, 1 UT8, 3ORY, 3Q8K, 3Q8L, 3Q8M, 3UVU, 3ZD8, 3ZD9, 3ZDA, 3ZDB, 3ZDC, 3ZDD, 3ZDE, 4WA8, 5E0V, 5FV7, SHML, SHMM, SHNK, 5HP4, 5K97, SKSE, 5T9J and 5UM9; available online at www(dot)rcsb(dot)org).

Useful flap endonucleases include eukaryotic flap endonucleases, including e.g., mammalian flap endonucleases, yeast flap endonucleases (e.g., Saccharomyces cerevisiae (UniProt P26793, RefSeq. NP_012809.1), Schizosaccharomyces pombe (UniProt P39750, RefSeq. NP_594972.1), etc.), invertebrate flap endonucleases (e.g., fly (UniProt Q9VRJ0, RefSeq. NP_647943.2), nematode (UniProt Q9N3T2, RefSeq. NP_491168.1), etc.), plant flap endonucleases (e.g., rice (UniProt Q9SXQ6, RefSeq. XP_015639321.1), Arabidopsis (UniProt O65251, RefSeq. NP_001154740.1), etc.), and the like. Non-limiting examples of mammalian flap endonucleases include e.g., human flap endonucleases (e.g., UniProt P39748, RefSeq. NP_004102.1), mouse flap endonucleases (e.g., UniProt P39749, RefSeq. NP_001258544.1), rat flap endonucleases (e.g., UniProt Q5XIP6, RefSeq. NP_445882.1), bovine flap endonucleases (e.g., UniProt Q58DH8, RefSeq. NP_001030285.1), and the like.

Useful flap endonucleases include prokaryotic flap endonucleases, including e.g., bacterial flap endonucleases (e.g., E. coli flap endonucleases (e.g., UniProt P38506, RefSeq. NP_417278.4), Salmonella flap endonucleases (e.g., UniProt P39369, RefSeq. WP_001312504.1), etc.), and the like. Useful flap endonucleases include archaea flap endonucleases, including e.g., Halobacterium flap endonucleases (e.g., UniProt Q9HQ27, RefSeq. WP_010902986.1), Thermococcus flap endonucleases (e.g., UniProt Q5JGN0, RefSeq. WP_011250232.1), Pyrobaculumand flap endonucleases (e.g., UniProt Q8ZYN2, GenBank AE009441), Archaeoglobus flap endonucleases (e.g., Archaeoglobus fulgidus FEN1, UniProt O29975, RefSeq. WP_010877775.1), and the like.

In some instances, a useful flap endonuclease may be FEN-1, including wild-type and recombinantly modified versions thereof. Useful flap endonucleases include thermostable and thermolabile flap endonucleases, including e.g., thermostable FEN-1, thermolabile FEN-1, and the like. Useful flap endonucleases include but are not limited to commercially available flap endonucleases including e.g., thermostable FEN1 available from New England Biolabs, Inc. (Ipswich, Mass.), and the like.

Useful final concentrations of the components of a cleavage reaction involving a junction complex and a flap endonuclease will vary. In some instances, a flap endonuclease enzyme may be employed at a final concentration in a subject reaction mixture ranging from less than 0.1 mg/mL to 0.5 mg/mL or more, including but not limited to e.g., 0.1 to 0.4 mg/mL, 0.1 to 0.3 mg/mL, 0.1 to 0.2 mg/mL, 0.2 to 0.5 mg/mL, 0.2 to 0.4 mg/mL, 0.2 to 0.3 mg/mL, 0.3 to 0.4 mg/mL, etc. In some instances, a suitable concentration of flap endonuclease may be determined empirically, e.g., as based on one or more of the following criteria: Z-factor (e.g., equal to or higher than 0.6), ratio between positive and negative (e.g., equal to or higher than 6) and/or ratio between the positive and the background (e.g., equal to or higher than 3).

The stock and final (i.e., reaction) concentrations of the various nucleic acid-based reagents (e.g., flap probe, displacer oligonucleotide, detection cassettes, etc.) will also vary depending on particular reaction conditions and the edit to be detected. For example, the final concentration in a subject reaction mixture of individual nucleic acid-based components may range from less 10 nM to 10 μM or more, including but not limited to e.g., 10 nM to 1 μM, 10 nM to 750 nM, 10 nM to 500 nM, 10 nM to 100 nM, 50 nm to 100 nM, 100 nM to 1 μM, 200 nM to 1 μM, 300 nM to 1 μM, 400 nM to 1 μM, 500 nM to 1 μM, 600 nM to 1 μM, 700 nM to 1 μM, etc.

Kits of the present disclosure may include one or more detection cassettes. Such kits may include a single detection cassette or multiple. For example, in some instances, a kit may include multiple detection cassettes that bind the same 5′ flap region but produce different signals (e.g., signals of different colors or wavelengths) to allow for user selection of the signal produced in the reaction. In some instances, kits may include one or more pairs of detection cassette and flap probe, where such pairs may be compatible (i.e., the detection cassette detects the released 5′ flap region of the probe). Kits may be configured to include two or more pairs of detection cassette and flap probe, where such pairs may be identifiable in a reaction by different detectable signals.

Kits may include detection components specifically designed to identify an edit and a lack of an edit at the same target site (i.e., identify the edited and unedited target site). Components for identifying the edit site may be configured to produce a first signal and components for identifying the unedited site may be configured to produce a second signal, where the first and second signals are able to be differentiated (e.g., by color, wavelength, etc.).

Kits may include one or more reagents for performing control reactions or reference standard reagents. Useful controls include positive controls (e.g., cells or nucleic acids known to contain the desired edit at a target site of a control target nucleic acid, including flap probes specific for the positive controls, displacer oligonucleotides specific for the positive controls, etc.). In some instances, positive controls may confirm the presence of an edited nucleotide at an edit site of a control target nucleic acid. Useful positive controls may also include essentially any nucleic acid with an edit, including a complete or partial insertion edit, a complete or partial deletion edit, and the like. Useful controls include negative controls (e.g., cells or nucleic acids known not to contain the desired edit at a target site of a control target nucleic acid, etc.). Negative controls useful in the kits of the present disclosure may include essentially any nucleic acid with a partial or absent edit, including a partial or absent insertion edit, a partial or absent deletion edit, and the like. In some instances, negative controls may confirm the absence of the edited nucleotide. In some instances, negative controls may confirm the presence of an undesired edit, a partial edit, or the like. Experimental reagents (i.e., gene editing reagents for which efficacy is unknown) may, in some instances, be evaluated in comparison to controls. Experimental reagents may, in some instances, be evaluated internally, e.g., by comparison relative to other experimental reagents.

Kits may include certain combinations of components in a single reaction vessel. For example, the single container kit may include an endonuclease and a detection cassette. The kit may further include a gene-editing reagent. The single container may further include a control nucleic acid, such as control target nucleic acid comprising the edited nucleotide at the edit site or a negative control nucleic acid comprising an unedited edit site. A kit may further include a single container containing a displacer oligonucleotide, and a flap probe. In some instances, one or more of these components may be present separately (i.e., in a separate container within the kit). In some instances, certain nucleic acid reagents may be supplied by or separately ordered/acquired by a user. For example, a user may separately acquire displacer oligonucleotide and/or flap probe, e.g., displacer oligonucleotide and/or flap probe directed to a specific target site and/or edit of interest to the user.

Kits may include different components of different detection strategies, including where the different components are provided to allow a user to select between two or more detection strategies for a particular reaction. For example, in some instances, a kit may include two or more different flap probes or two or more different detection cassettes compatible with two at least two different detection strategies. In some instances, among two or more different flap probes at least one of the two or more different flap probes blocks the activity of the flap endonuclease when hybridized to its target site. In some instances, among two or more different flap probes at least one of the two or more different flap probes comprises a 5′ flap region that has a sequence configured, when released from the at least one flap probe, to not hybridize to the detection cassette. In some instances, where two or more different detection cassettes are provided, the two or more different detection cassettes may include at least two different detectable signal producing systems (e.g., systems of different types (e.g., chromogenic and fluorescent, etc.), systems of different color or wavelength (e.g., two different fluorescence emission wavelengths, etc.), and the like).

Kits may include or exclude various other components, including but not limited to e.g., an amplification polymerase buffer, an annealing buffer, an endonuclease buffer, a nucleic acid dilution buffer, an amplification polymerase, a nucleic acid extraction reagent, a cell lysis reagent or a combination thereof. In some instances, a kit may include instructions for producing or assembling one or more of the above described components. In some instances, a provided annealing buffer may include exogenous DNA or a kit may include annealing buffer and exogenous DNA separately with instructions to combine them in a reaction mixture. Subject kits may include a nucleic acid extraction reagent and a cell lysis reagent, where such reagents may be in different or the same container.

Kits may further include one or more reagents for gene editing, including e.g., a component of a Clustered Regularly Interspaced Short Palindromic Repeat (CRISPR)/nuclease system (including e.g., a guide RNA (gRNA), a nuclease, etc.), a component of a Transcription Activator-Like Effector Nuclease (TALEN) system (including e.g., a TAL effector DNA-binding domain containing protein, etc.), a component of a Zinc-Finger Nuclease (ZFN) system (e.g., including e.g., a ZFN, etc.), and the like. In some instances, the kits may further include one more components for carrying out or enhancing the rate of HDR. In some instances, a kit may include a plurality of gene editing reagents, including e.g., a plurality of one or a combination of reagents of the systems described herein.

In addition to the above components, the subject kits may further include (in some embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), portable flash drive, Hard Drive etc., on which the information has been recorded. Yet another form of these instructions that may be present is a website address which may be used via the internet to access the information at a removed site.

The following example(s) is/are offered by way of illustration and not by way of limitation.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and Takara.

### Example 1: Assay Workflow

An assay for the detection of a desired single nucleic acid edit made in a target genome of a cell was developed. Genomic DNA was extracted from the interrogated cells and used as a template in a PCR reaction. The PCR product was then hybridized with two different complementary probes: the displacer oligo (that hybridizes 5′ from the interrogated base and has an extra base in the 3′) and the flap probe (that hybridizes with the interrogated base, extends 3′ with a targeting region that is complementary to the target nucleic acid sequence 5′ adjacent to the target site and has a non-complementary sequence in the 5′ flap region forming a flap structure). FEN-1 recognizes complete base-pairing between the flap probe and the template in the formed three-dimensional structure and subsequently cleaves this flap. This cleaved oligo acted in its turn as a displacer probe in a secondary reaction where the template and the flap probe are in the same oligonucleotide forming a hairpin structure (FRET cassette). The cleavage of the flap oligo in this second reaction released a fluorophore generating a fluorescence signal detected via plate reader. The flap probe and the displacer oligo are complementary to the target sequence and their Tm may be in the range of 60-63° C. and 70-72° C., respectively.

### Example 2: Determination of FEN-1 Concentration

The activity levels of FEN-1 can affect signal generated in the assay. The FEN-1 enzyme may be titrated to determine which concentration allows for a maximum ratio between the positive signal and the negative signal or the background signal (no template, NT).

Oligos encoding for the MTHFR gene encoding for the WT or mutation A1298C were used as templates for the reaction in 1 nM concentration. The oligo (10 ul at 1 nM) was mixed with 1 ul of displacer oligo (1 uM), 0.5 ul of flap probe (20 uM) and 3.5 ul of annealing buffer. The mixture was transferred to a PCR machine and the probes were annealed to the template with the following program:

Step 1. 95° C. 5 min

Step 2. From 95° C. to 63° C. at 0.1° C./s

Step 3. 63° C. for 10 min

Step 4. 63° C. forever

To each annealing reaction, 1 ul of FEN-1 was added in different concentrations with 0.5 ul of FRET cassette (10 uM) and 3.5 ul of reaction buffer. The mixture was incubated 1 h 15 minutes at 63° C. After the incubation time, water (35 ul) was added in each well and the fluorescence was detected using a plate reader (Excitation: 485 nm/Emission: 535 nm)

The most suitable concentration of FEN-1 fulfilled the following criteria:
Z-factor equal to or higher than 0.6, where the Z-factor is calculated as:

$$Z factor = 1 - \frac{3 ( {σ}_{p} + {σ}_{n} )}{\vert {μ}_{p} - {μ}_{n} \vert} ,$$

where μₚ, σₚ, μₙ and σₙ are the means (μ) and standard deviations (σ) of the positive (p) and negative (n) controls.
Ratio between positive and negative equal to or higher than 6
Ratio between the positive and the background equal to or higher than 3

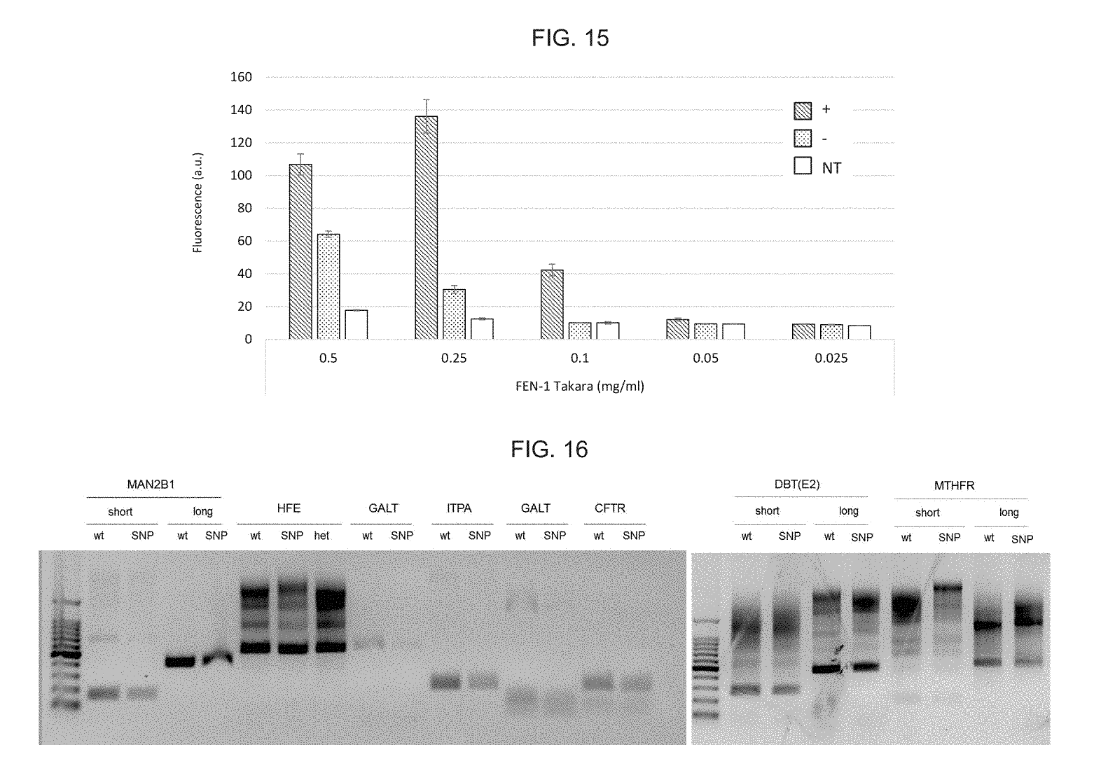

As shown in FIG. 15, the Takara FEN-1 as reference enzyme produced a strong difference in signal to noise between positive and negative control at 0.25 mg/mL. This example demonstrates that a suitable concentration of FEN-1 for the assay may be determined through titration of a control reaction.

### Example 3: DNA Fragment Size

The assay was tested using starting amplicons of varying size to assess the influence of this factor on reaction success. As a template, 10 ng of genomic DNA (obtained from Coriell repositories) was amplified using Terra polymerase with the following the program:

| | | | | |
|---|---|---|---|---|
| 1 | cycle | 98° C. | 2 | min |
| 32 | cycles | 98° C. | 10 | s |
| | | 60° C. | 15 | s |
| | | 68° C. | 1 | min |

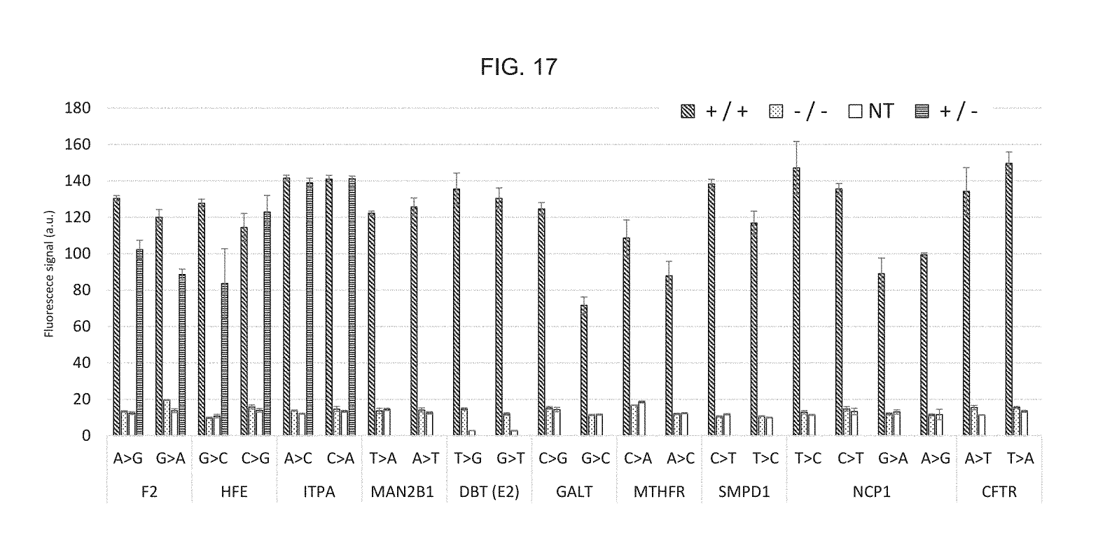

After PCR amplification, the samples were analyzed via agarose gel to visualize the produced amplicon sizes. As shown in FIG. 16, the fragments range from 170 bp to 650 bp and had different degrees of purity. All fragments were used for the detection of different transitions in various genomic loci. All of differently sized fragments were successfully used as templates for detection using FEN-1 (as shown in FIG. 16). This demonstrates that a wide range of DNA fragment sizes may be employed in the assay.

### Example 4: Templates Homogenous and Heterogeneous at the Target Site

The performance of the assay in the presence of homogenous and heterogeneous template was assessed using various SNPs in human genes as model single nucleotide edits. As a template, 10 ng of genomic DNA (obtained from Coriell repositories) was amplified using Terra polymerase as described in Example 3.

After PCR amplification, PCR products were diluted 1/40 and used as templates in the detection of SNPs using FEN-1. The diluted PCR product was mixed with 1 ul of displacer oligo (1 uM), 0.5 ul of flap probe (20 uM) and 3.5 ul of annealing buffer. The mixture was transferred to a PCR machine and the probes were annealed to the template with the following program:

Step 1. 95° C. 5 min

Step 2. From 95° C. to 63° C. at 0.1° C./s

Step 3. 63° C. for 10 min

Step 4. 63° C. forever

Next the reaction with FEN-1 enzyme was performed. To each annealing reaction, 1 ul of FEN-1 in different concentrations was added with 0.5 ul of FRET cassette (10 uM) and 3.5 ul of reaction buffer. This mixture was incubated 1 h 15 minutes at 63° C. After incubation, water (35 ul) was added in each well and the fluorescence was detected using a plate reader (Excitation: 485 nm/Emission: 535 nm). All transitions were successfully detected, even in the cases where the sample was heterozygous for the SNP (FIG. 17). This demonstrates the effectiveness of the assay to detect a desired edit regardless of whether the sample is homogenous or heterogeneous with respect to the target site.

### Example 5: Analyzing Human SNPs as a Proxy for Single Nucleotide Change Detection

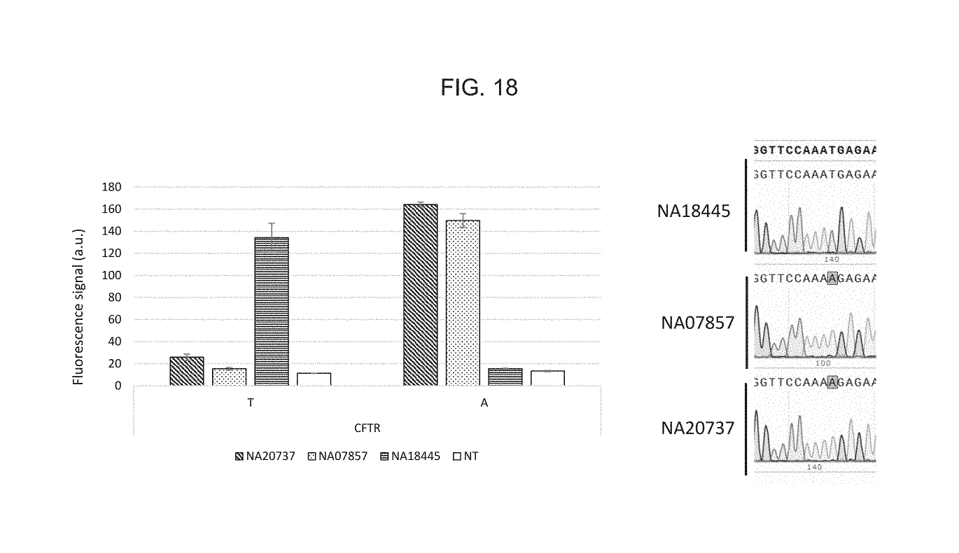

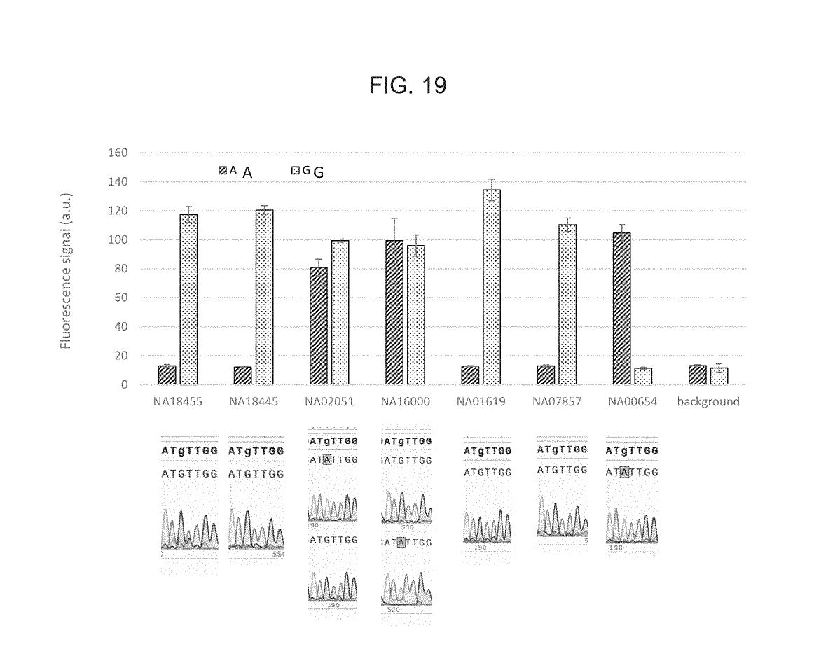

The herein described FEN-1-based assay was further evaluated using various human SNPs as a proxy for single nucleotide edits, such as for genotyping a sample. As shown in FIG. 18, in the case of CFTR mutation, the FEN-1 method of the disclosure was able to detect that the sample (from Coriell NA20737) had a T-to-A transition at nucleotide 3434 (3434T>A) which converts the met-1101 codon (ATG) to a lys (AAG), resulting in a missense mutation in exon17b in the CFTR gene. The fluorescence levels in this case were similar to the ones obtained using sample NA07857 which is reported in the Coriell database to have the same T>A transition. The assay also detected that the sample NA18445 was wild type (T) in that position. Both results were confirmed by Sanger sequencing as also depicted in FIG. 18.

The methods of the disclosure were also able to genotype different samples by interrogating their homozygosity or heterozygosity. FIG. 19 shows interrogation of the mutation A>G at nucleotide 2572 (c.2572A>G) in gene NCP1 in different samples. Only the sample NA18445 was reported to carry this mutation in the database (positive control). The assay was able to determine which samples had a G or A as well as their heterozygosity using two independent experimental flap probes designed to detect G or A. As shown in FIG. 19, samples NA02051 and NA16000 were heterozygous, while all other samples were homozygous. Sample NA00654 was detected in the FEN-1 assay to be WT with an A at the relevant site, which was confirmed by Sanger sequencing, also shown in FIG. 19. The bolded sequences in the sanger sequencing plots indicate the reference sequence and the unbolded sequences in the sanger sequencing plots indicate the sample sequence. Using human SNPs as a proxy for edited nucleic acids, these data further demonstrate the ability of the assay of the subject disclosure to detect single nucleotide changes in a target genomic DNA, and thereby genotype a sample

### Example 6: Application of the Detection Assay in Cells

Human SNPs were used as a proxy for single nucleotide edits to assess the application of the detection assay in cells. Fibroblasts that carried different transitions in various genes (obtained from Coriell repositories) were plated at different cell numbers ranging from 16.50E5 to 0.01E5 cells per well in 96-well plate. Each transition has two different cell lines, one carrying the desired SNP and the other one being wild type on that site. After 48 hours, genomic DNA was extracted using MightyPrep (Takara Bio USA, Mountain View, Calif.) reagent. Cells were washed with PBS, 50 ul of MightyPrep reagent was added followed by incubation at 95° C. for 10 minutes. Afterwards, the plate was centrifuged and the supernatant was transferred to another plate.

As a template, 2 ul of the genomic DNA was amplified using Terra polymerase following the program:

| | | | | |
|---|---|---|---|---|
| 1 | cycle | 98° C. | 2 | min |
| 32 | cycles | 98° C. | 10 | s |
| | | 60° C. | 15 | s |
| | | 68° C. | 1 | min |

With the following mix:
2 ul of genomic DNA
12.5 ul of Buffer
0.5 ul of polymerase
0.75 ul of each primer (10 uM)
8.5 ul of water

After PCR amplification, PCR products were diluted 1/40 and used as templates in the detection of SNPs using FEN-1. The diluted PCR product was mixed with 1 ul of displacer oligo (1 uM), 0.5 ul of flap probe (20 uM) and 3.5 ul of annealing buffer. The mixture was transferred to a PCR machine and the probes were annealed to the template with the following program:

Step 1. 95° C. 5 min

Step 2. From 95° C. to 63° C. at 0.1° C./s

Step 3. 63° C. for 10 min

Step 4. 63° C. forever

The next step was the reaction with FEN-1 enzyme. To each annealing reaction, 1 ul of FEN-1 in different concentrations was added with 0.5 ul of FRET cassette (10 uM) and 3.5 ul of reaction buffer. This mixture was incubated 1 h 15 minutes at 63° C.

After the incubation time, water (35 ul) was added in each well and the fluorescence was detected using a plate reader (Excitation: 485 nm/Emission: 535 nm).

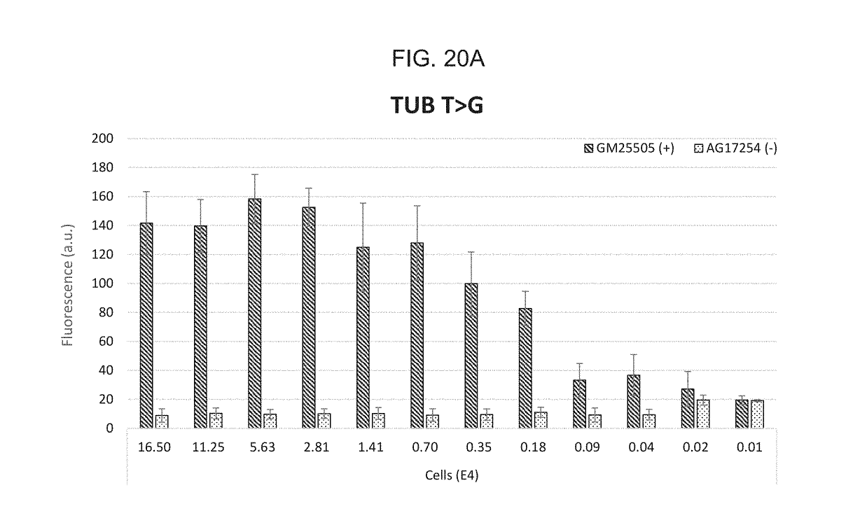

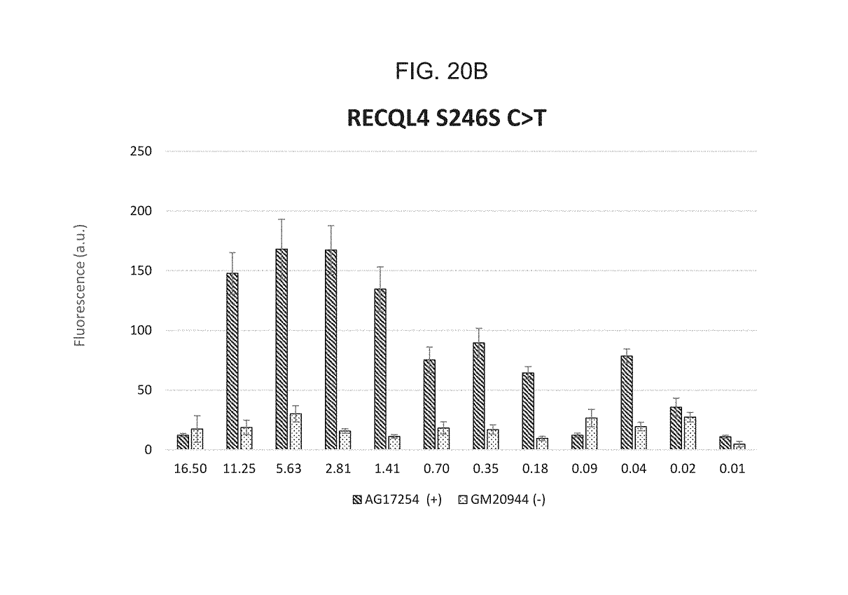

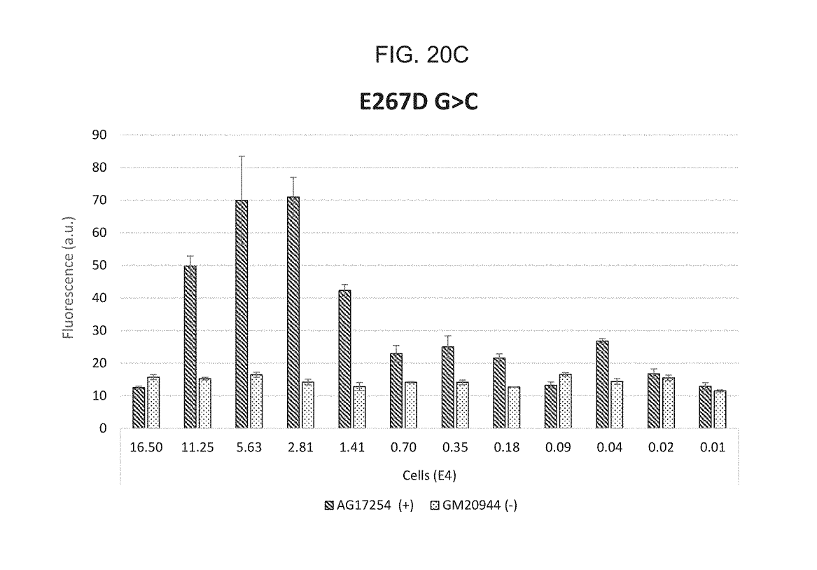

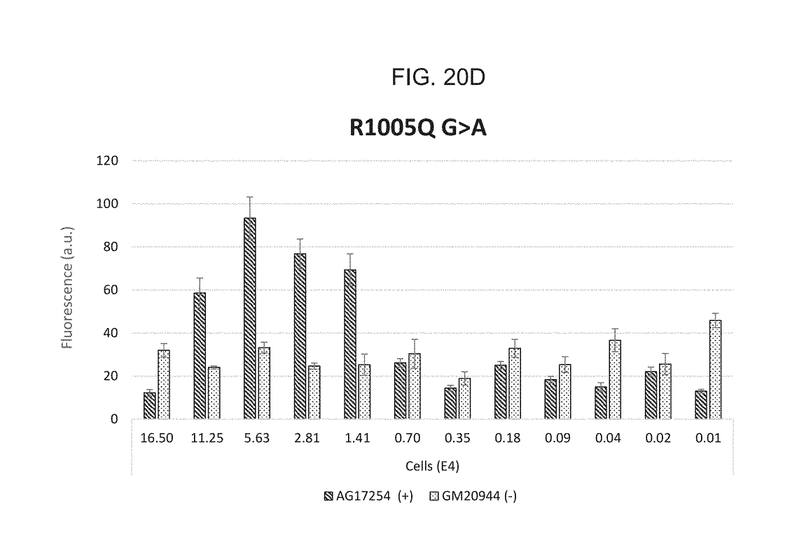

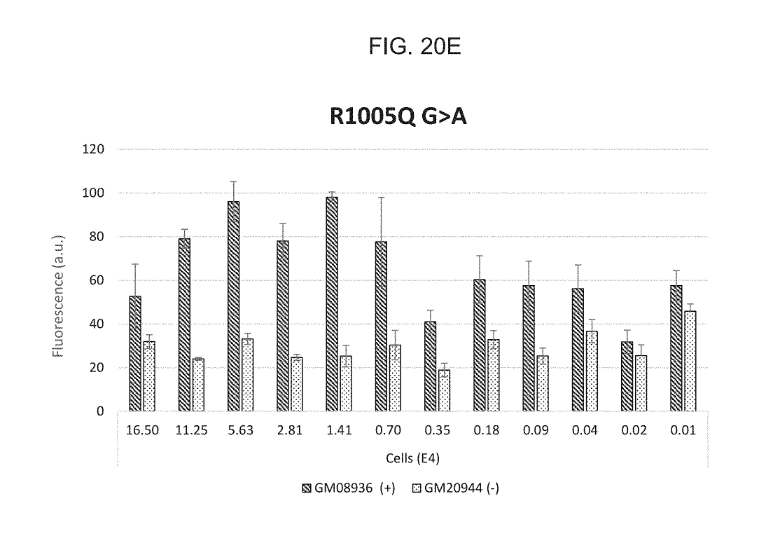

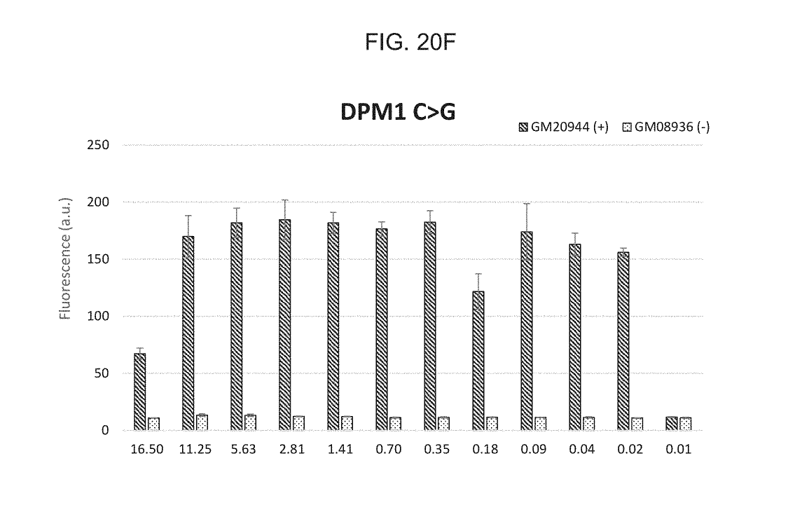

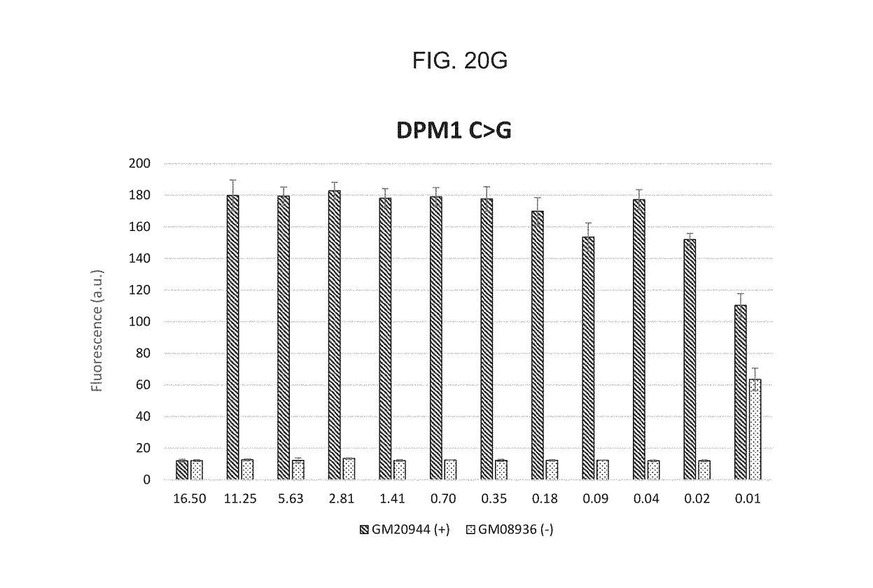

Each experiment was performed in triplicates. The range of cell number that gave the best results was between 11.25E4 to 0.7E4 for various SNPs and genes (see FIG. 20A-20G; FIG. 20F and FIG. 20G were produced using templates of different lengths, 700 bp and 300 bp, respectively). For example, as shown in FIG. 20A, the highest signal was achieved with a cell number ranging from 16.50E4 to 0.7E4. This example demonstrates the application of the detection assay from human cells cultured at various starting plating numbers.

### Example 7: Assay Sensitivity

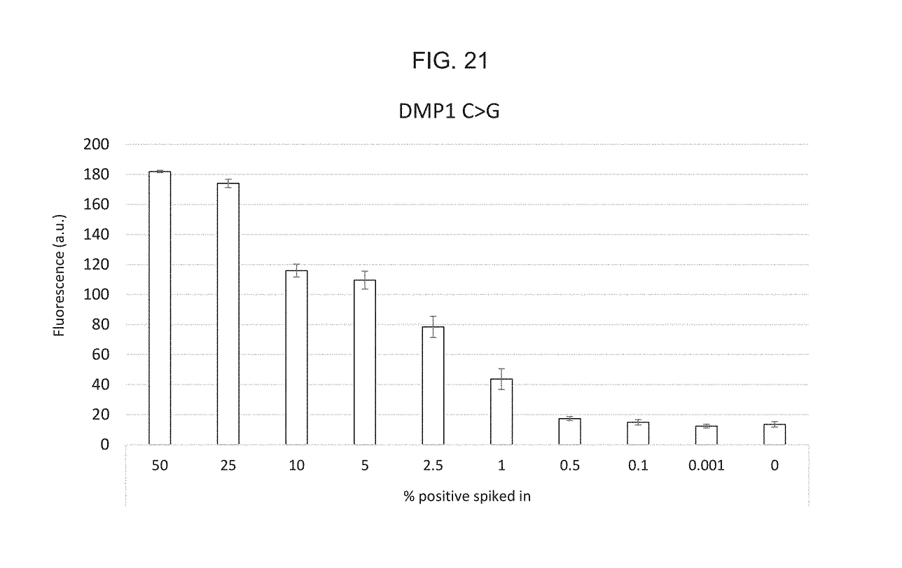

The sensitivity of the assay was evaluated taking DMP1 C>G transition SNP as a test case to serve as a proxy for a single nucleotide edit. Different percentages of PCR diluted product having a G at the transition site (G/PCR) (acting as positive sample) were spiked in a PCR diluted product having a C at the transition site (C/PCR) (acting as negative sample). Following the protocol described in FIG. 21, the G nucleotide at the target site was detected in the different C/PCR diluted product samples containing varying percentages of spiked in G/PCR diluted product. As it shown in the graph (FIG. 21), the method can detect positive sample spiked into negative sample at percentages as low as 1%.

### Example 8: Dual Detection

The FEN-1-based detection assay was configured for dual detection of two different nucleotides at a target site within a genome. Dual-detection of both bases was performed using FRET cassettes with different emission wavelengths. Oligos encoding for the Prothrombin gene encoding for the WT or mutation G20210A were used as templates for the reaction in 1 nM concentration. The oligo (10 ul at 1 nM) was mixed with 1 ul of displacer oligo (1 uM), 0.5 ul of flap probe designed to detect G (20 uM) and with a flap sequence complementary to a FRET cassette with emission in the green channel, 0.5 ul of flap probe designed to detect A (20 uM) and with a flap sequence complementary to a FRET cassette with emission in the red channel and 3 ul of annealing buffer. The mixture was transferred to a PCR machine and the probes were annealed to the template with the following program:

Step 1. 95° C. 5 min

Step 2. From 95° C. to 63° C. at 0.1° C./s

Step 3. 63° C. for 10 min

Step 4. 63° C. forever

Next, the FEN-1 enzyme was added to the reaction. Specifically, to each annealing reaction, 1 ul of FEN-1 in different concentrations was added with 0.5 ul of the green channel emission FRET cassette (10 uM), 0.5 ul of the red channel emission FRET cassette (10 uM) and 3 ul of reaction buffer. This mixture was incubated 1 h 15 minutes at 63° C.

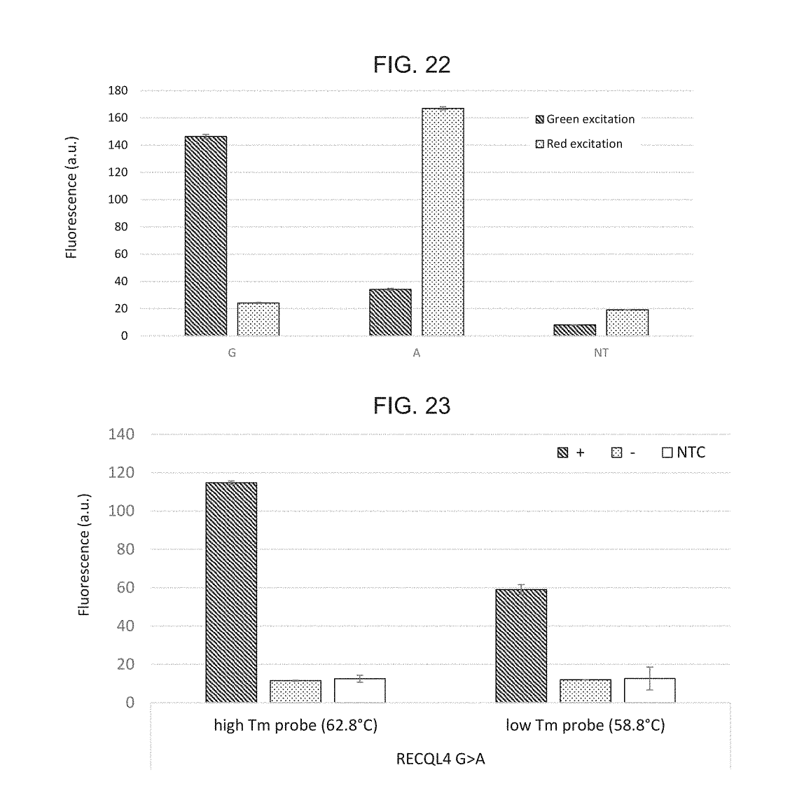

After the incubation, water (35 ul) was added in each well and the fluorescence was detected using a plate reader (Excitation: 485 nm/Emission: 535 nm for the green channel and Excitation: 540 nm/Emission: 577 nm). As shown in FIG. 22, both the green and red channels detected the specific SNPs corresponding to each nucleotide. This example demonstrates effective simultaneous dual detection of two different single nucleotide changes using detection cassettes with different wavelengths.

### Example 9: Detection Using Flap Probes with Various Tm's

Following the protocol described in Example 4, the RECQL4 transition G>A was detected using flap probes with various different Tm's. Efficient detection was achieved using probes with Tm's above 58.8° C., as exemplified by the 62.8° C. Tm probe shown in FIG. 23. When the utilized flap probe had a lower Tm of 58.8° C., the fluorescence signal decreased, reducing the ratio of positive vs negative signal.

### Example 10: Increased Signal with Addition of Exogenous DNA

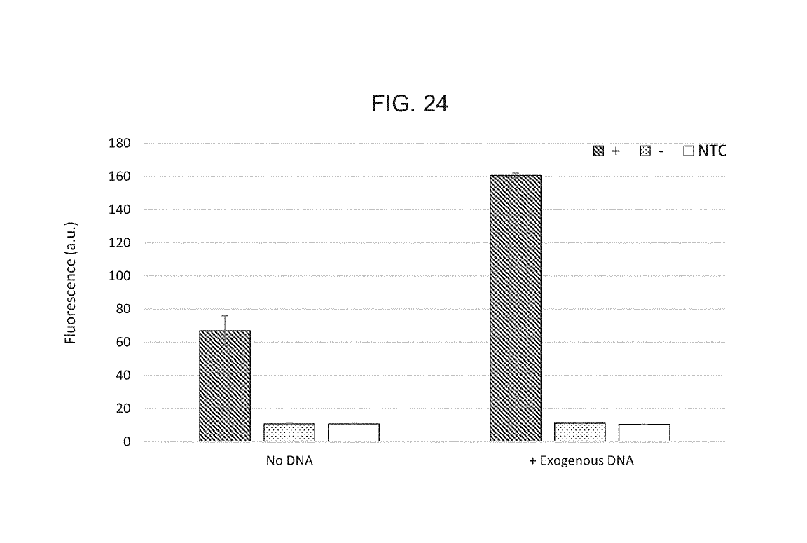

Following the protocol described in Example 4, the SMPD1 transition T>C was detected in parallel with and without exogenous DNA added to the annealing buffer (FIG. 24). Where employed, 5 ng/ul of exogenous DNA added to the annealing buffer increased the obtained positive signal and therefore the ratio between positive and negative signal. This example demonstrates that the addition of exogenous DNA in the annealing buffer can increase the fluorescence signal in the positive samples, resulting in an increased positive-to-negative signal ratio.

### Example 11: Increased Signal-to-Noise Using Cleavage-Blocking Flap Probes

FEN-1 activity can be inhibited by DNA secondary structure if trinucleotide repeats are present in the flap probe. Thus, the effect of adding a second cleavage-blocking flap probe that targets an unedited nucleotide target site was tested following the protocol previously described herein. As a test case, an A>G (negative>positive) transition at the position 20210 in the prothrombin gene was used.

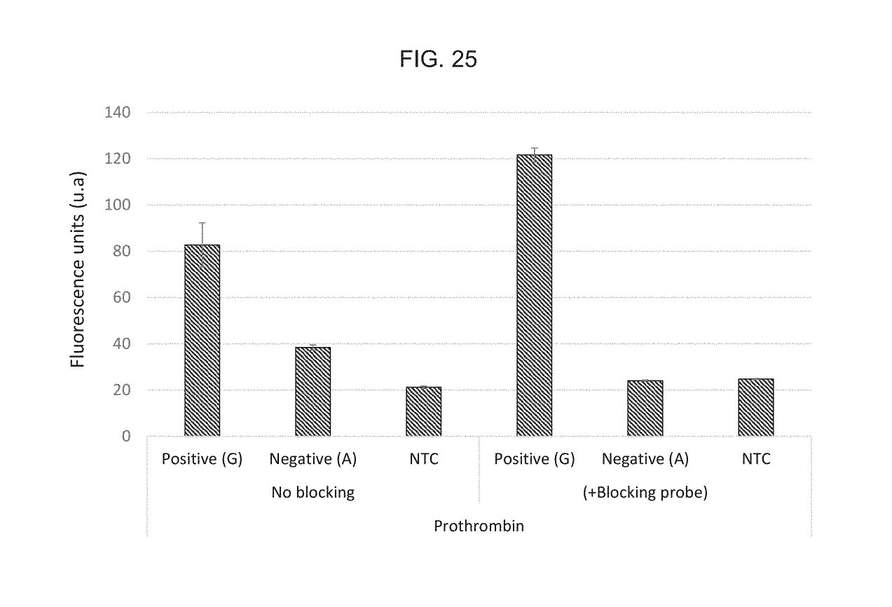

As shown in FIG. 25, two distinct flap probes were employed. The first one targeted the G target site (termed “Positive (G)”) and had a standard sequence as described herein. The second flap probe targeted the A target site (termed “Negative (A)”) and had extra trinucleotide repeats that inhibit FEN-1 activity. The flap probe targeting the G was tested without (“No blocking”, lanes 1-3) and with (“+Blocking probe”, lanes 4-6) the second flap probe against A added in a 1:3 ratio. Fluorescence produced as a result from the generation free flap probe against G was measured in each reaction. As shown in FIG. 25, when the second flap probe that included secondary structure blocking cleavage and that targeted the A target site was added, the difference between the positive signal (G) and the negative signal (A) was increased as compared to where no blocking probe against A was employed.

This example demonstrates that the addition of secondary structure blocking cleavage of a flap probe, targeted to an unedited target site, can increase the positive signal and decrease the negative signal, resulting in an increased positive-to-negative signal ratio.

### Example 12: Detection of Heterozygosity by Dual Detection Cassettes

Two detection cassettes with two different fluorophores were used to detect whether alleles in a sample are homozygous WT, homozygous edited with a SNP, or heterozygous. The Prothrombin gene was amplified from three different genomic DNA samples (Coriell) encoding for homozygous (G or A) or heterozygous (G+A) at the position 20210. The PCR product was mixed with 1 ul of displacer oligo (1 uM), 0.5 ul of flap probe designed to detect G (20 uM) and comprising a flap sequence complementary to a FRET cassette with emission in the green channel, 0.5 ul of flap probe designed to detect A (20 uM) and comprising a flap sequence complementary to a FRET cassette with emission in the red channel, and 3 ul of annealing buffer.

The mixture was transferred to a PCR machine and the probes were annealed to the template with the following program:

Step 1. 95° C. 5 min

Step 2. From 95° C. to 63° C. at 0.1° C./s

Step 3. 63° C. for 10 min

Step 4. 63° C. for ever

In the next step the reaction with FEN-1 enzyme was performed. To each annealing reaction, 1 ul of FEN-1 in different concentrations was added with 0.5 ul of FRET cassette with green channel emission (10 uM), 0.5 ul of FRET cassette with red channel emission (10 uM) and 3 ul of reaction buffer. This mixture was incubated 1 h 15 minutes at 63° C. After the incubation, water (35 ul) was added in each well and the fluorescence was assayed using a plate reader (Excitation: 485 nm/Emission: 535 nm for the green channel and Excitation: 561 nm/Emission: 612 nm for the red channel).

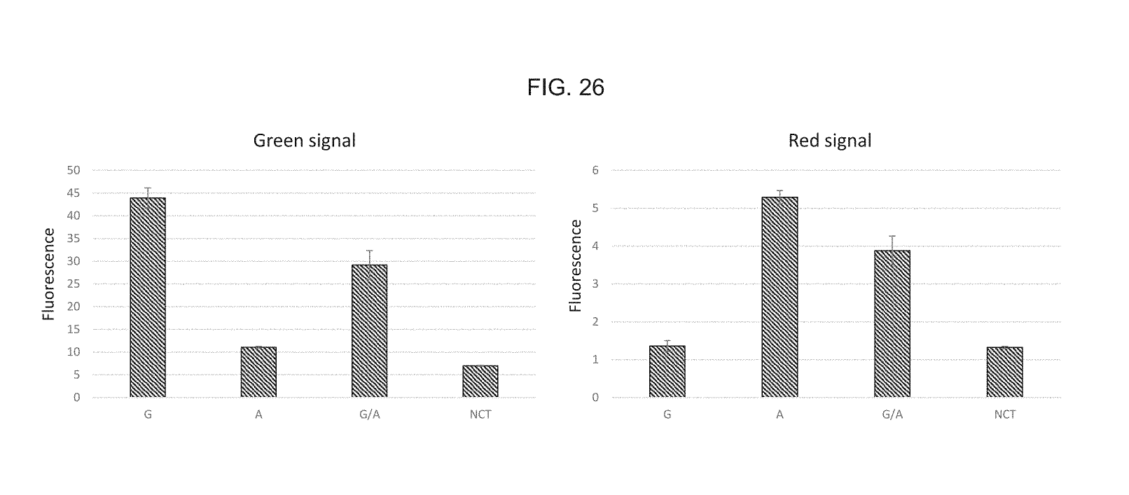

As shown in FIG. 26, samples homozygous for either G or A showed a higher signal in either the green or red channels respectively. The sample that was heterozygous for G/A showed positive signals in both the green and red channels. This assay shows that the use of two different detection cassettes can be employed to identify sample heterozygosity and sample homozygosity. (NTC=no template control).

### Example 13: Workflows for the Detection of Single-Base Substitutions or Longer Knock-in Insertions

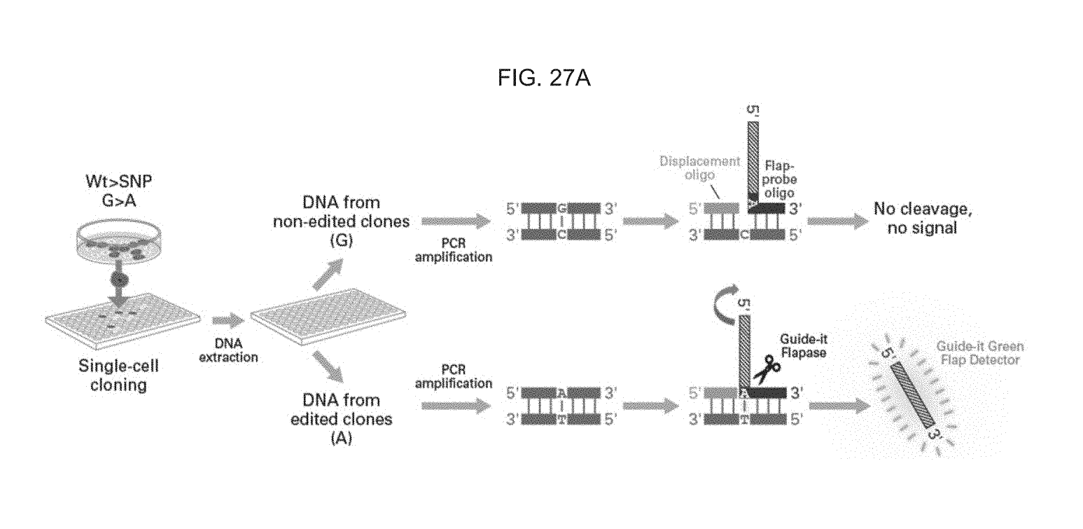

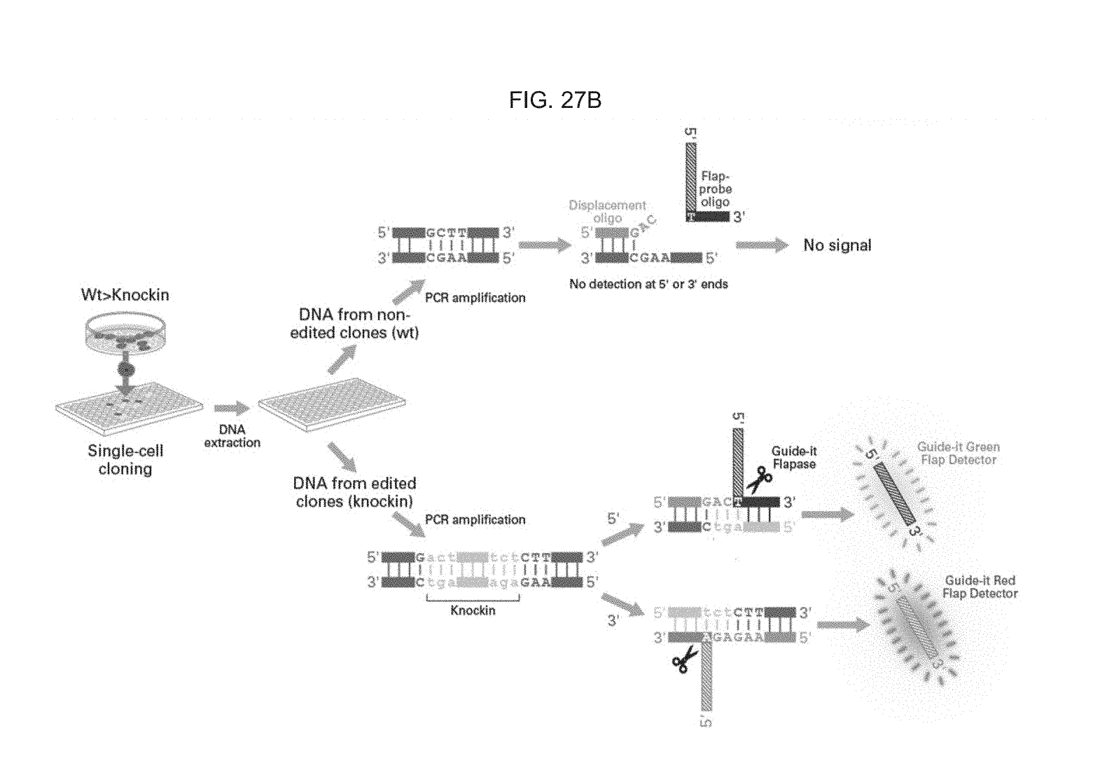

Schematics depicting non-limiting examples of a workflow for the detection of single-base substitutions (FIG. 27A) and a workflow for the detection of longer knock-in insertions (FIG. 27B) are provided.

The example workflow depicted in FIG. 27A shows the detection of a G>A transition (where G is the wild-type base edited to an A). After the genome editing event, single cells are isolated via FACS or limiting dilution and expanded to clonal cell lines that can be either wild-type or successfully edited. After DNA extraction and subsequent PCR amplification of the target site, the PCR product is annealed with two different complementary oligo probes—the displacement oligo and the flap-probe oligo that are designed to hybridize with the target site in the region adjacent to the interrogated base (defined as the actual SNP base for which screening is performed). After the annealing of the two DNA oligos with the PCR product, Guide-it Flapase recognizes a complete base-pairing between the flap-probe oligo and the PCR product in the resulting three-dimensional structure and subsequently cleaves the flap. This cleavage event causes the release of the flap oligo, which is detected downstream by the Guide-it Flap Detector, generating a fluorescence signal that can be measured using a plate reader.

As depicted in FIG. 27B, for the detection of longer knock-ins, the PCR product is annealed with two different sets of displacement and flap-probe oligos: one set that hybridizes with the 5′ end of the insert, and the other with the 3′. Each flap-probe oligo has a specific fixed and distinct flap sequence that allows for the generation of a green or a red fluorescence signal. If the homologous recombination (HR) event has been successful and seamless, the full hybridization of the probes at both ends (5′ and 3′) will generate both green and red fluorescence signals after the cleavage of the respective flap oligos by the Guide-it Flapase. Detection of only one signal (e.g., red or green) would indicate an insertion truncated on either the 5′ or 3′ end, respectively.

### Example 14: Detection of SNP in Heterogenous Edited Samples and Clonal Cell Lines

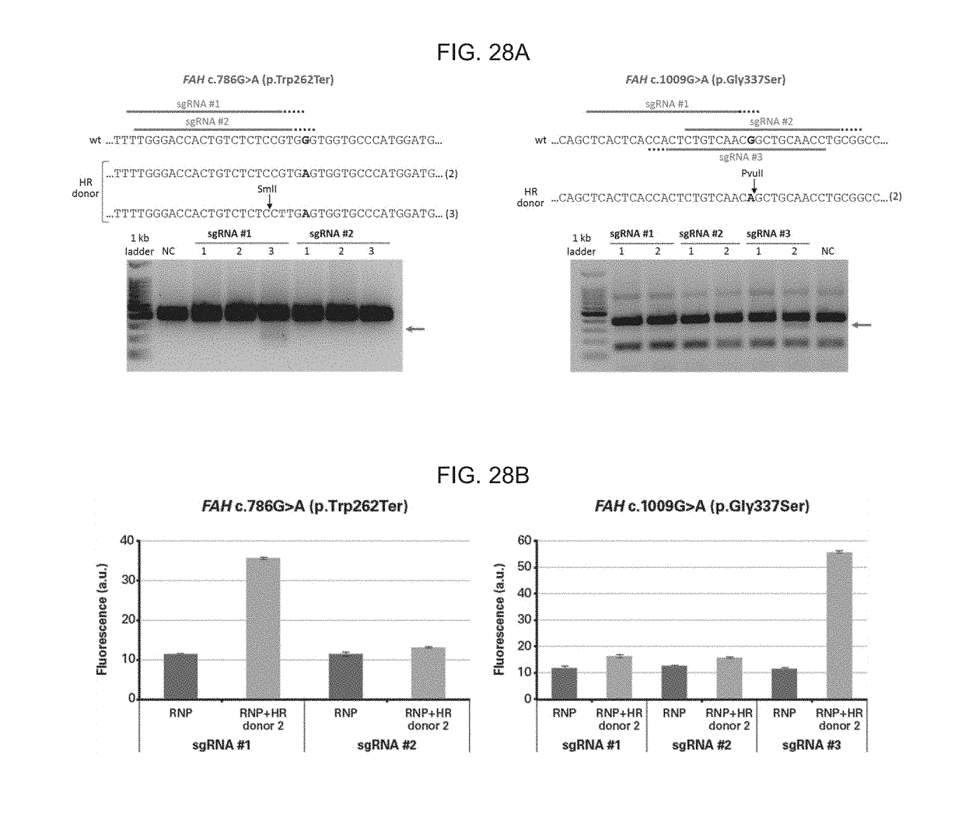

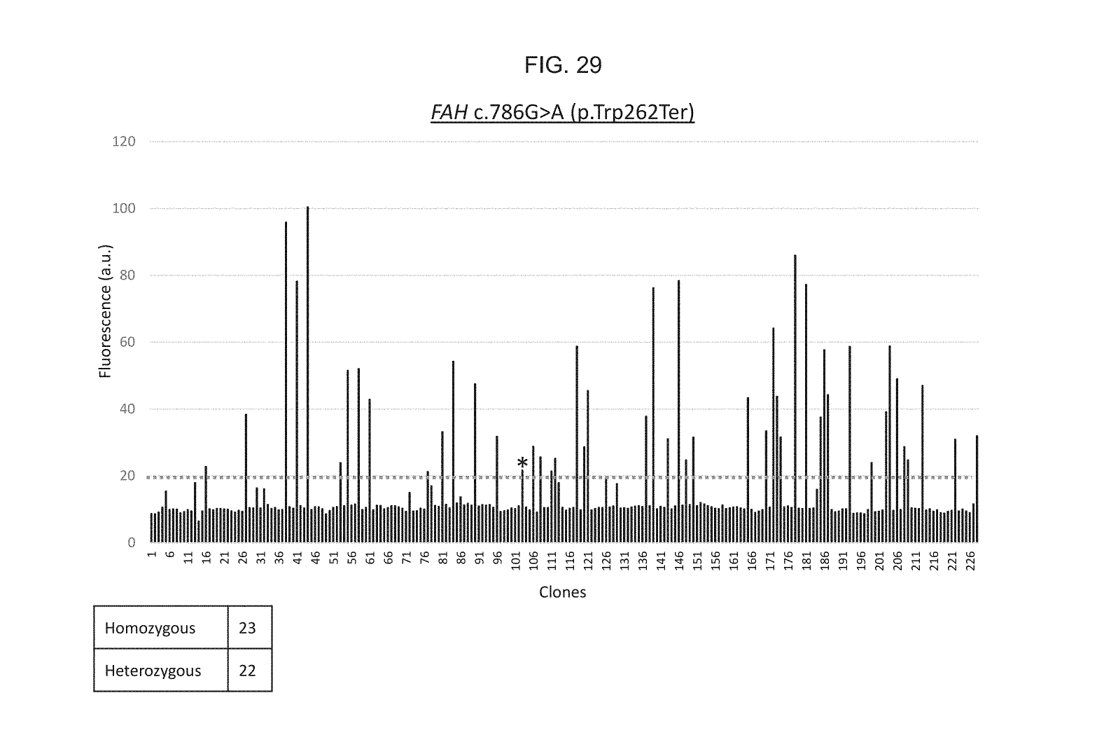

Detection of tyrosinemia-related SNPs in human induced pluripotent stem cells (hiPSCs) using the Guide-it SNP Screening Kit is shown in FIG. 28A-28B.

In FIG. 28A two SNPs in the FAH locus related to tyrosinemia (p.Gly337Ser and p.Trp262Ter) were generated in cells from hiPSCs by electroporation of the Cas9 RNP complex together with a short oligo encoding the SNP acting as the homologous recombination (HR) donor. This editing is schematized in the nucleic acid sequences provided. Different single guide RNAs (sgRNAs) (indicated by #1, #2 and #3) were tested and an Restriction Fragment Length Polymorphism (RFLP) assay was used to detect the HR in the pool of edited cells (see gel images provided). In the case of FAH Gly337Ser, the edited base (shown in bold) introduced a new restriction site for the Pvull enzyme; in the case of Trp262Ter, a second template (HR donor 3) with another base mutation (shown in small caps) was used to generate a new restriction site for the enzyme Smll. In each case, the HR event could be detected in only one of the sgRNAs used (as seen in the gel images—the band of interest is marked with an arrow); (NC) negative control; (1) cells electroporated with RNP with no HR donor; (2) RNP+HR donor 2; (3) RNP+HR donor 3 with an extra base change encoding a restriction site.

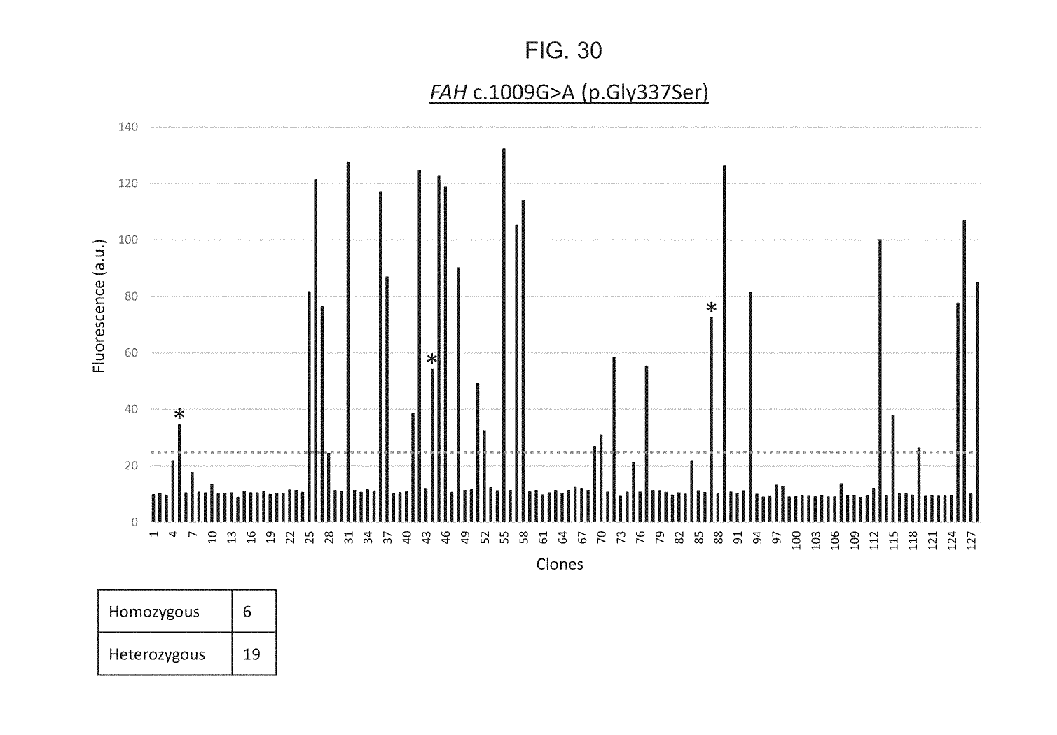

In FIG. 28B the Guide-it SNP Screening Kit was also used to detect the successful HR event in the pool of edited cells before single-cell isolation. After extraction of the genomic DNA with column purification, and amplification of the target region by PCR, the DNA sample was hybridized with a displacement oligo and a flap-probe oligo designed to detect the SNP. In both cases, the fluorescent signal correlated with the result obtained by the RFLP assay. Single cells from the FAH c.786G>A RNP+HR donor 2 sgRNA#1 population and the FAH c.1009G>A RNP+HR donor 2 sgRNA#3 population were isolated and analyzed as shown in FIG. 29 and FIG. 30, respectively.

With reference to FIG. 29, single cells were isolated by limiting dilution and expanded. Forty-five days after seeding, clonal cell lines were interrogated for the respective SNPs. In each case, approximately 19-24% of the clonal cell lines generated a positive fluorescent signal. The correlation between the fluorescence above a specific detection signal (dotted line) and the existence of the SNPs in the interrogated base was confirmed by Sanger sequencing in all the tested clonal cell lines to be homo- or heterozygous. Non-clonal samples are marked with an asterisk.

With reference to FIG. 30, single cells were isolated by limiting dilution and expanded. Forty-five days after seeding, clonal cell lines were interrogated for the respective SNPs. In each case, approximately 19-24% of the clonal cell lines generated a positive fluorescent signal. The correlation between the fluorescence above a specific detection signal (dotted line) and the existence of the SNPs in the interrogated base was confirmed by Sanger sequencing in all the tested clonal cell lines to be homo or heterozygous. Non-clonal samples are marked with an asterisk.

Collectively, these data demonstrate the detection of SNPs in heterogenous edited samples containing pooled cells as well as produced clonal cell lines. Moreover, SNP detection by the described assay was independently verified in both cases by RFLP assay and Sanger sequencing.

### Example 15: Detection of Large Insertions in a Heterogenous Edited Population and Clonal Cell Lines

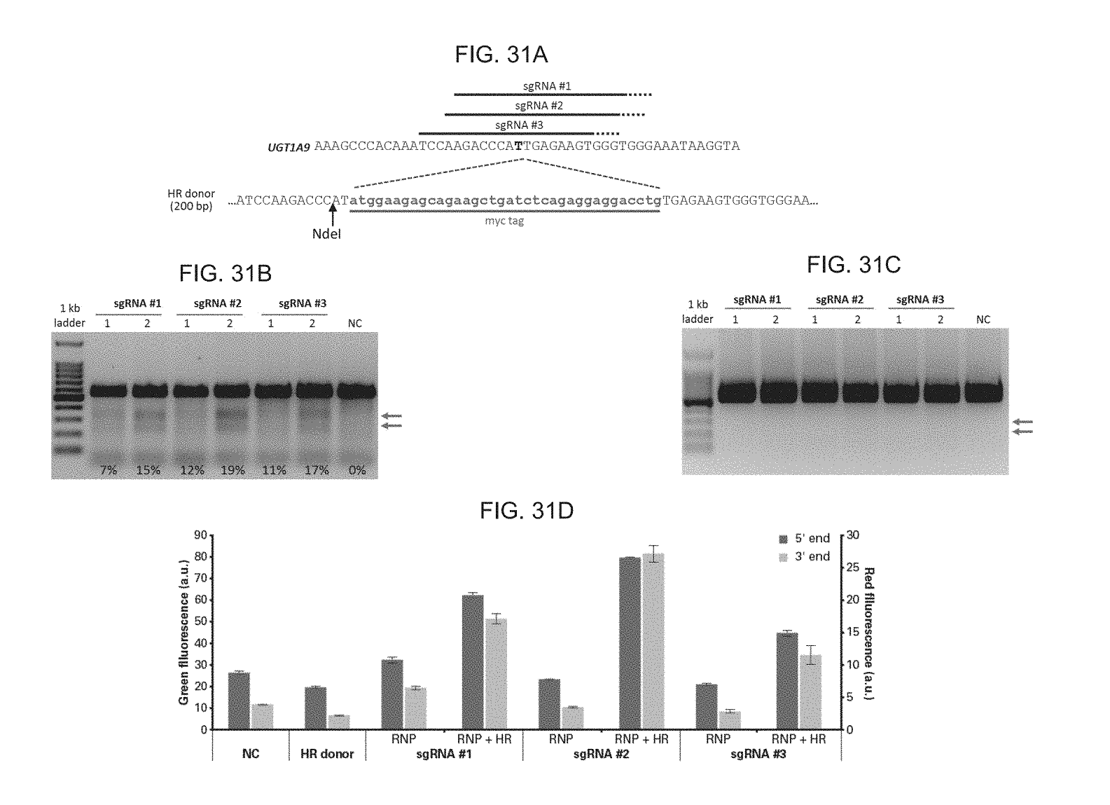

Detection of the tagging of the UGT1A9 gene with a myc tag in hiPSCs is demonstrated with reference to FIGS. 31A-31D and FIGS. 32-33.

The editing strategy employed is schematized in FIG. 31A. HiPSCs were electroporated with Cas9 RNP complex together with an oligo encoding for a myc tag flanked by homology arms allowing for integration at the 3′-end of the UGT1A9 gene. Three different sgRNAs (#1, #2, and #3) targeting an area around the insertion site (see bold thymidine in UGT1A9 sequence) were tested. Insertion of the myc tag introduces a NdeI restriction enzyme recognition site upstream of the insertion site (indicated by an arrow).

Guide-it Mutation Detection Kit was used to detect editing events at the target site: (1) RNP; (2) RNP+ donor; (NC) Negative control as shown in FIG. 31B. The bands of interest are marked with arrows. For comparison purposes, detection of the desired insertion was performed using an RFLP assay. As shown in FIG. 31C, no successful HR events could be detected in any case when the RFLP assay was used to detect the knock-in (KI) in the pool of edited cells (the arrows indicate the theoretical position of the undetected bands).

As depicted in FIG. 31D, the Guide-it KI Screening Kit was used to detect the successful, full-length HR events in the pool of edited cells. After extraction of genomic DNA and amplification of the target region by PCR, the DNA samples were hybridized with two sets of probes designed to detect the presence of correct insertion at the 5′ and 3′ ends. As can be seen in the data, the highest signal was obtained from the population electroporated with an RNP complex containing sgRNA #2. This population was chosen for the subsequent single-cell clone isolation.

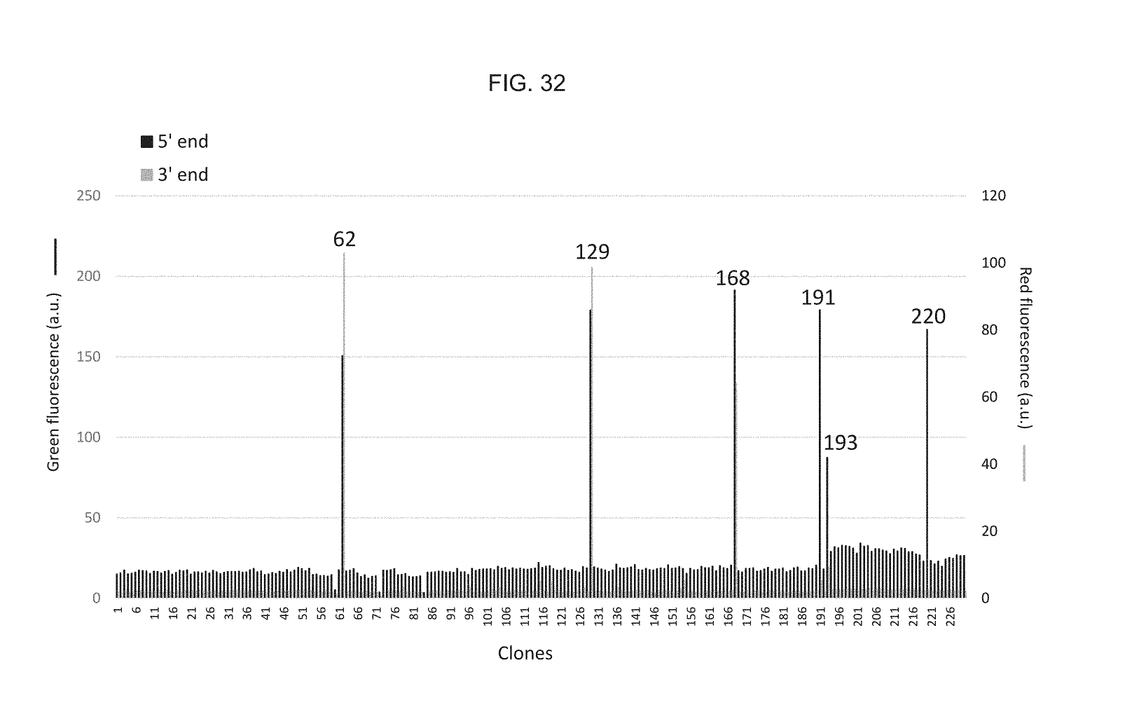

For the single-cell clone isolation and detection depicted in FIG. 32, single cells were isolated by flow cytometry and expanded. Thirty days after seeding, clonal cell lines were interrogated for myc-tag insertion using the Guide-it KI Screening Kit. Out of 230 clones, only three were positive for a correct insertion at both ends (green and red fluorescence could be detected in clones 62, 129 and 168, indicated in FIG. 32). Three other clones only gave a positive signal related to a correct 5′ insertion (i.e., only a green signal could be detected in clones 191, 193 and 220, indicated in FIG. 32).

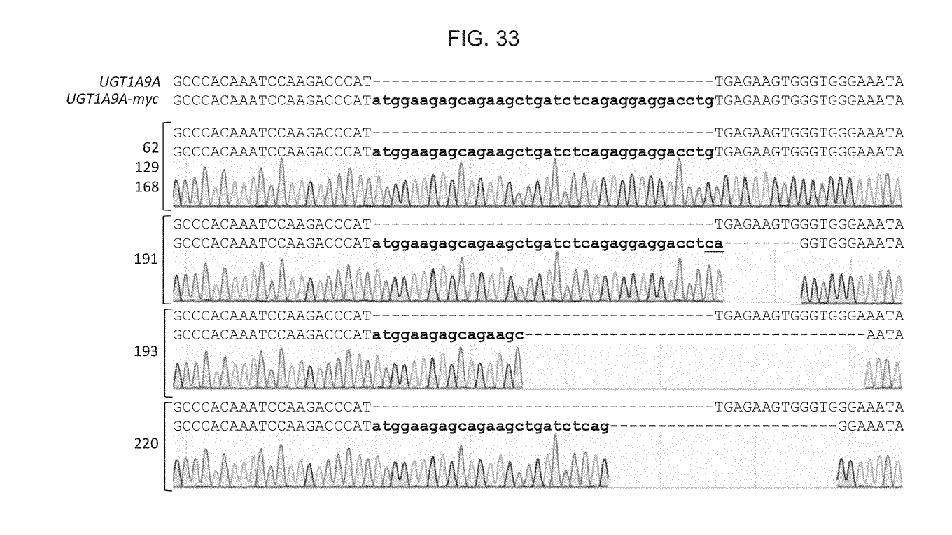

The results from the Guide-it KI Screening kit were corroborated by Sanger sequencing as shown in FIG. 33. The three positive clones (62, 129 and 168) were heterozygous for the insertion (lowercase) with one allele encoding for the wild-type sequence. No homozygous clone for the insertion was obtained. The other three clones (191, 193 and 220) were also heterozygous with an allele encoding for the wild-type and the other allele encoding a truncated myc tag (extra bases are underlined).

Collectively, these data demonstrate the detection of large insertions in heterogenous edited samples of pooled cells as well as produced clonal cell lines. In addition, RFLP analysis was insufficient for detection in this case. Moreover, using the dual-end (i.e., paired 5′-end and 3′-end insertion) detection strategy allowed for the successfully differentiation of correctly edited loci (i.e., complete and proper insertion of the myc tag encoding sequence) as compared to incorrectly edited loci (i.e., truncated insertions and insertions containing spurious extra base insertions) as independently verified by Sanger sequencing.

Notwithstanding the appended claims, the disclosure is also defined by the following clauses:

1. A method of identifying whether a cell that has been contacted with a gene editing reagent has been edited at a target site, the method comprising:
a) combining nucleic acids from the cell, or an amplification product thereof, with:
i) a displacer oligonucleotide comprising a 5′ targeting region that is complementary to a sequence of the nucleic acid 3′ adjacent to the target site; and
ii) a flap probe comprising a 3′ targeting region that is complementary to a sequence of the nucleic acid 5′ adjacent to the target site and a 5′ flap region comprising a hinge nucleotide;
into a reaction mixture under conditions sufficient to produce a junction complex comprising the nucleic acid or amplification product thereof, the displacer oligonucleotide and the flap probe, wherein hybridization of the flap probe 3′ targeting region to the nucleic acid aligns the hinge nucleotide with the target site;
b) contacting the reaction mixture with a flap endonuclease under conditions sufficient to release the 5′ flap region from the junction complex when the hinge nucleotide is complementary to the edited target site; and
c) assaying for the released 5′ flap region, wherein presence of the released 5′ flap region indicates that the cell has been edited at the target site and absence of the released 5′ flap region indicates that the cell has not been edited at the target site.

2. The method according to Clause 1, further comprising obtaining the cell from a plurality of cells that have been contacted with the gene editing reagent.

3. The method according to Clause 2, wherein obtaining the cell comprises cell sorting.

4. The method according to Clauses 2 or 3, wherein the cell is a clone produced from the plurality.

5. The method according to Clause 4, further comprising producing the clone.

6. The method according to any of the preceding clauses, further comprising lysing the cell prior to the combining.

7. The method according to any of the preceding clauses, wherein the combining and the contacting are performed in a reaction vessel of a multi-vessel device.

8. The method according to any of Clauses 2 to 7, further comprising combining into a plurality of reaction mixtures, each in a separate reaction vessel:
nucleic acids from a cell of the plurality, or an amplification product thereof;
the displacer oligonucleotide; and
the flap probe.

9. The method according to Clause 8, further comprising identifying whether each cell of the plurality has been edited at the target site.

10. The method according to any of the preceding clauses, further comprising amplifying the nucleic acids or a portion thereof comprising the target site.

11. The method according to Clause 10, wherein the nucleic acids or the portion thereof is amplified prior to the combining.

12. The method according to any of the preceding clauses, further comprising isolating the nucleic acids prior to the combining.

13. The method according to any of the preceding clauses, wherein the nucleic acids are genomic nucleic acids.

14. The method according to any of the preceding clauses, wherein the flap endonuclease is a FEN1 flap endonuclease.

15. The method according to any of the preceding clauses, wherein the target site comprises a disease associated allele.

16. The method according to any of Clauses 1 to 14, wherein the target site does not comprise a disease associated allele.

17. The method according to any of the preceding clauses, wherein the edit comprises a mutation.

18. The method according to Clause 17, wherein the mutation is selected from the group consisting of: a single nucleotide change, an insertion, a deletion or a combination thereof.

19. The method according to Clause 18, wherein the mutation comprises an insertion of a coding sequence.

20. The method according to Clause 19, wherein the coding sequence is a heterologous coding sequence.

21. The method according to any of the preceding clauses, further comprising contacting the cell with the gene editing reagent.

22. The method according to any of the preceding clauses, wherein the gene editing reagent is a component of a Clustered Regularly Interspaced Short Palindromic Repeat (CRISPR)/nuclease system.

23. The method according to Clause 22, wherein the CRISPR/nuclease system comprises a guide RNA (gRNA).

24. The method according to any of Clauses 1 to 21, wherein the gene editing reagent is a component of a Transcription Activator-Like Effector Nuclease (TALEN) system.

25. The method according to Clause 24, wherein the TALEN system comprises a TAL effector DNA-binding domain.

26. The method according to any of Clauses 1 to 21, wherein the gene editing reagent is a component of a Zinc-Finger Nuclease (ZFN) system.

27. The method according to Clause 26, wherein the ZFN system comprises a ZFN.

28. The method according to any of the preceding clauses, wherein assaying for the released 5′ flap region comprises assaying for a detectable signal.

29. The method according to Clause 28, wherein the detectable signal is a fluorescent signal.

30. The method according to Clause 29, wherein the flap probe comprises a quenched fluorophore and the fluorescent signal results from unquenching of the quenched fluorophore.

31. The method according to Clause 29, wherein the fluorescent signal is generated when the released 5′ flap region hybridizes with a detection cassette, comprising a quenched fluorophore, to form a junction complex that is cleaved by the flap endonuclease, thereby unquenching the quenched fluorophore.

32. The method according to any of the preceding clauses, wherein the reaction mixture is contacted with a second flap probe comprising a 3′ targeting region and a 5′ flap region comprising a hinge nucleotide that is complementary to the unedited target site.

33. The method according to Clause 32, wherein the second flap probe is unable to be cut by the flap endonuclease when hybridized to the unedited target site.

34. The method according to Clause 32, wherein the 5′ flap region of the second flap probe has a sequence that is configured to not hybridize, when released, with a detection cassette present in the reaction mixture.

35. The method according to Clause 32, wherein the 5′ flap region of the second flap probe has a sequence that is configured to hybridize to a detection cassette, comprising a quenched fluorophore, to form a junction complex that is cleaved by the flap endonuclease, thereby unquenching the quenched fluorophore.

36. The method according to any of the preceding clauses, wherein the reaction mixture comprises:
a first detection cassette that hybridizes with the 5′ flap region that, when released, indicates that the cell has been edited at the target site; and
a second detection cassette that hybridizes with a 5′ flap region of a second flap probe that, when released, indicates that the cell has not been edited at the target site.

37. The method according to Clause 36, wherein the first detection cassette, prior to hybridizing with the released 5′ flap probe, comprises a quenched fluorophore of a first wavelength and the second detection cassette, prior to hybridizing with the released 5′ flap probe, comprises a quenched fluorophore of a second wavelength.

38. The method according to Clause 37, wherein detection of a fluorescent signal at the first wavelength indicates that the cell has been edited at the target site and detection of a fluorescent signal at the second wavelength indicates that the cell has not been edited at the target site.

39. The method according to any of the preceding clauses, wherein the flap probe comprises an extension preventing moiety.

40. The method according to any of the preceding clauses, wherein the method further comprises maintaining a separate clone of the cell.

41. The method according to Clause 1, wherein the method further comprises repeating steps (a) through (c) to identify whether the cell has been edited to comprise a second edit at a second target site.

42. The method according to Clause 41, wherein repeating the method comprises a displacer oligonucleotide and a flap probe each specific to the second target site and configured for determining whether the cell comprises the second edit at the second target site by assaying for a released 5′ flap region of the flap probe specific for the second target site.

43. A method of selecting an edited clone, the method comprising:
a) contacting a plurality of cells with a gene editing reagent;
b) generating a plurality of clones of individual cells of step (a); and
c) identifying whether a clone of step (b) has been edited at a target site according to the method of any of Clauses 1 to 42 to select an edited clone.

44. The method according to Clause 43, wherein the method further comprises assaying a phenotype of the selected edited clone.

45. The method according to Clause 43, wherein the method further comprises assaying a phenotype of a cell of the plurality prior to step a) or step b).

46. The method according to Clause 44, wherein the assayed phenotype is compared to a corresponding phenotype in a corresponding unedited cell.

47. The method according to any of Clauses 44 to 46, wherein the phenotype comprises a response to a stimulus.

48. The method according to Clause 47, wherein the stimulus is a pharmacological agent.

49. The method according to any of Clauses 43 to 48, wherein the plurality of cells is a plurality of prokaryotic cells.

50. The method according to Clause 49, wherein the plurality of prokaryotic cells comprise bacteria.

51. The method according to any of Clauses 43 to 48, wherein the plurality of cells is a plurality of eukaryotic cells.

52. The method according to Clause 51, wherein the plurality of eukaryotic cells comprise a culture of unicellular eukaryotes.

53. The method according to Clause 52, wherein the culture of unicellular eukaryotes comprises unicellular fungi or unicellular algae.

54. The method according to Clause 51, wherein the plurality of eukaryotic cells is a primary cell culture derived from a multicellular organism.

55. The method according to Clause 51, wherein the plurality of eukaryotic cells is an immortalized cell line derived from a multicellular organism.

56. The method according to Clause 51, wherein the method comprises collecting the plurality of eukaryotic cells from a multicellular organism that was administered the gene editing reagent.

57. The method according to any of Clauses 54 to 56, further comprising incorporating the selected edited clone into a multicellular host.

58. The method according to Clause 57, wherein the multicellular host is an embryo.

59. The method according to any of Clauses 53 to 58, wherein the multicellular organism is selected from the group consisting of: an invertebrate, a vertebrate, a plant, and a fungus.

60. A method of determining whether a polyploid cell, that has been contacted with a gene editing reagent, contains an edit at a target site, an unedited target site or both, the method comprising:
a) combining nucleic acids from the cell, or an amplification product thereof, with:
i) a displacer oligonucleotide specific for the target site;
ii) a first flap probe specific for the edit at the target site and comprising a first 5′ flap region;
iii) a second flap probe specific for the unedited target site and comprising a second 5′ flap region;
into a reaction mixture under conditions sufficient to produce first and second junction complexes comprising the displacer oligonucleotide and either the first flap probe annealed to the edited target site or the second flap probe annealed to the unedited target site;
b) contacting the reaction mixture with:
i) a flap endonuclease under conditions sufficient to release the first and second 5′ flap regions from the first and second junction complexes;
and
c) assaying for the first and second 5′ flap regions, wherein detection of the first 5′ flap region indicates that the polyploid cell contains the edit at the target site, detection of the second 5′ flap region indicates that the polyploid cell contains the unedited the target site, and detection of both the first and second 5′ flap regions indicates that the polyploid cell contains both the edit at the target site and the unedited target site.

61. The method according to Clause 60, wherein the first flap probe when cleaved by the flap endonuclease produces a first detectable signal and the second flap probe when cleaved by the flap probes produces a second detectable signal.

62. The method according to Clause 61, wherein the first flap probe comprises a first fluorophore and the second flap probe comprises a second fluorophore.

63. The method according to clause 60, wherein the contacting further comprises contacting the reaction mixture with a first detection cassette specific for the released first 5′ flap region and configured to produce a first detectable signal and a second detection cassette specific for the released second 5′ flap region and configured to produce a second detectable signal.

64. The method according to any of Clauses 61 to 63, wherein the first detectable signal and the second detectable signal are fluorescent signals of different wavelengths.

65. The method according to any of Clauses 60 to 64, wherein the edit is a single nucleotide change.

66. The method according to any of Clauses 60 to 65, wherein the gene editing reagent is a component of a Clustered Regularly Interspaced Short Palindromic Repeat (CRISPR)/nuclease system.

67. The method according to Clause 66, wherein the CRISPR/nuclease system comprises a guide RNA (gRNA).

68. The method according to any of Clauses 60 to 65, wherein the gene editing reagent is a component of a Transcription Activator-Like Effector Nuclease (TALEN) system.

69. The method according to Clause 68, wherein the TALEN system comprises a TAL effector DNA-binding domain.

70. The method according to any of Clauses 60 to 65, wherein the gene editing reagent is a component of a Zinc-Finger Nuclease (ZFN) system.

71. The method according to Clause 70, wherein the ZFN system comprises a ZFN.

72. The method according to any of Clauses 60 to 71, wherein the method further comprises maintaining a separate clone of the polyploid cell.

73. The method according to any of Clauses 60 to 72, wherein the polyploid cell is a diploid cell.

74. The method according to Clause 73, wherein the diploid cell is a mammalian cell.

75. A method of assessing the efficacy of a gene editing reagent, the method comprising:
a) combining a nucleic acid that has been contacted with the gene editing reagent that is configured to produce an edit at a target site with:
i) a displacer oligonucleotide comprising a 5′ targeting region that is complementary to a sequence of the nucleic acid 3′ adjacent to the target site; and
ii) a flap probe comprising a 3′ targeting region that is complementary to a sequence of the nucleic acid 5′ adjacent to the target site and a 5′ flap region comprising a hinge nucleotide;
into a reaction mixture under conditions sufficient to produce a junction complex comprising the nucleic acid, the displacer oligonucleotide and the flap probe, wherein hybridization of the flap probe 3′ targeting region to the nucleic acid aligns the hinge nucleotide with the target site;
b) contacting the reaction mixture with a flap endonuclease under conditions sufficient to release the 5′ flap region from the junction complex when the hinge nucleotide is complementary to the edited target site; and
c) assaying for the released 5′ flap region to assess the efficacy of the gene editing reagent.

76. The method according to Clause 75, further comprising amplifying the nucleic acid or a portion thereof comprising the target site.

77. The method according to Clause 76, wherein the nucleic acid or the portion thereof is amplified prior to the combining.

78. The method according to any of Clauses 75 to 77, further comprising isolating the nucleic acid prior to the combining.

79. The method according to any of Clauses 75 to 78, wherein the flap endonuclease is a FEN1 flap endonuclease.

80. The method according to any of Clauses 75 to 79, wherein the target site comprises a disease associated allele.

81. The method according to any of Clauses 75 to 80, wherein the edit comprises a mutation.

82. The method according to any of Clauses 75 to 81, further comprising contacting the nucleic acid with the gene editing reagent.

83. The method according to any of Clauses 75 to 82, wherein the gene editing reagent is a component of a Clustered Regularly Interspaced Short Palindromic Repeat (CRISPR)/nuclease system.

84. The method according to Clause 83, wherein the CRISPR/nuclease system comprises a guide RNA (gRNA).

85. The method according to any of Clauses 75 to 82, wherein the gene editing reagent is a component of a Transcription Activator-Like Effector Nuclease (TALEN) system.

86. The method according to Clause 85, wherein the TALEN system comprises a TAL effector DNA-binding domain.

87. The method according to any of Clauses 75 to 82, wherein the gene editing reagent is a component of a Zinc-Finger Nuclease (ZFN) system.

88. The method according to Clause 87, wherein the ZFN system comprises a ZFN.

89. The method according to any of Clauses 75 to 88, wherein assaying for the released 5′ flap region comprises assaying for a fluorescent signal.

90. The method according to Clause 89, wherein the flap probe comprises a quenched fluorophore and the fluorescent signal results from unquenching of the quenched fluorophore.

91. The method according to Clause 89, wherein the fluorescent signal is generated when the released 5′ flap region hybridizes with a detection cassette, comprising a quenched fluorophore, to form a junction complex that is cleaved by the flap endonuclease, thereby unquenching the quenched fluorophore.

92. The method according to Clause 91, comprising contacting the reaction mixture with the detection cassette.

93. The method according to any of Clauses 75 to 82, wherein the reaction mixture further comprises a second flap probe comprising a 3′ targeting region and a 5′ flap region comprising a hinge nucleotide that is complementary to an unedited target site.

94. The method according to Clause 93, wherein the second flap probe is unable to be cut by the flap endonuclease when hybridized to the unedited target site.

95. The method according to Clause 93, wherein the 5′ flap region of the second flap probe has a sequence that is configured to not hybridize, when released, with a detection cassette present in the reaction mixture.

96. The method according to Clause 93, wherein the 5′ flap region of the second flap probe has a sequence that is configured to hybridize, when released, to a second detection cassette to generate a second fluorescent signal.

97. The method according to any of Clauses 93 to 96, wherein the method comprises comparing detection of the 5′ flap region released from the first flap probe with detection of the 5′ flap region released from the second flap probe.

98. The method according to any of Clauses 75 to 97, wherein the flap probe comprises an extension preventing moiety.

99. The method according to Clause 98, wherein the extension preventing moiety comprises a 3′ hexanediol.

100. The method according to any of Clauses 75 to 99, wherein the method assesses the efficacy of a plurality of gene editing reagents each combined into an individual reaction mixture present in an individual reaction vessel.

101. The method according to Clause 100, wherein the plurality of gene editing reagents all target the same target site.

102. The method according to Clauses 100 or 101, wherein the plurality of gene editing reagents are all configured to introduce the same edit.

103. The method according to any of Clauses 100 to 102, wherein the plurality of gene editing reagents comprise a plurality of different gRNAs.

104. The method according to any of Clauses 100 to 102, wherein the plurality of gene editing reagents comprise a plurality of different TAL effector DNA-binding domains.

105. The method according to any of Clauses 100 to 102, wherein the plurality of gene editing reagents comprise a plurality of different ZFNs.

106. The method according to any of Clauses 75 to 105, wherein the method is a non-diagnostic method.

107. A method of editing a nucleic acid, the method comprising:
a) assessing the efficacy of a plurality of gene editing reagents using the method according to any of Clauses 75 to 106;
b) selecting a gene editing reagent based on the assessed efficacy; and
c) editing the nucleic acid using the selected gene editing reagent.

108. The method according to Clause 107, wherein the nucleic acid is genomically integrated.

109. The method according to Clause 108, wherein the nucleic acid is integrated in a prokaryotic genome.

110. The method according to Clause 108, wherein the nucleic acid is integrated in a eukaryotic genome.

111. A kit comprising:
a flap endonuclease;
a detection cassette; and
a set of positive control nucleic acids that confirm the presence of an edited nucleotide at an edit site of a control target nucleic acid.

112. The kit according to Clause 111, wherein the set of positive control nucleic acids comprises a displacer oligonucleotide, a flap probe and the control target nucleic acid comprising the edited nucleotide at the edit site.

113. The kit according to Clauses 111 or 112, wherein the set of positive control nucleic acids are present in a single container.

114. The kit according to any of Clauses 111 to 113, further comprising a set of negative control nucleic acids that confirm the absence of the edit.

115. The kit according to Clause 114, wherein the set of negative control nucleic acids comprises a displacer oligonucleotide, a flap probe and a negative control nucleic acid comprising an unedited target site.

116. The kit according to Clauses 114 or 115, wherein the set of negative control nucleic acids are present in a single container.

117. The kit according to any of Clauses 111 to 116, wherein the flap endonuclease is a FEN1 flap endonuclease.

118. The kit according to any of Clauses 111 to 117, wherein the detection cassette comprises a fluorophore and a quencher.

119. The kit according to any of Clauses 111 to 118, wherein the flap probe comprises an extension preventing moiety.

120. The kit according to Clause 119, wherein the extension preventing moiety comprises a 3′ hexanediol.

121. The kit according to any of Clauses 111 to 120, further comprising an annealing buffer.

122. The kit according to any of Clauses 111 to 121, further comprising an endonuclease buffer.

123. The kit according to any of Clauses 111 to 122, further comprising a nucleic acid dilution buffer.

124. The kit according to any of Clauses 111 to 123, further comprising an amplification polymerase.

125. The kit according to any of Clauses 111 to 124, further comprising an amplification polymerase buffer.

126. The kit according to any of Clauses 111 to 125, further comprising a nucleic acid extraction reagent.

127. The kit according to any of Clauses 111 to 126, further comprising a cell lysis reagent.

128. The kit according to Clause 127, wherein the nucleic acid extraction reagent and the cell lysis reagent are in the same container.

129. The kit according to any of Clauses 111 to 128, wherein the kit further comprises two or more different flap probes for the set of positive control nucleic acids.

130. The kit according to any of Clauses 111 to 129, further comprising a second detection cassette.

131. The kit according to any of Clauses 111 to 130, further comprising instructions for identifying whether a cell that has been contacted with a gene editing reagent has been edited at a target site according to any of the methods of Clauses 1 to 42.

132. The kit according to any of Clauses 111 to 130, further comprising instructions for identifying whether a polypoid cell that has been contacted with a gene editing reagent contains an edit at a target site, an unedited target site or both according to any of the methods of Clauses 60 to 74.

133. The kit according to any of Clauses 111 to 130, further comprising instructions for assessing the efficacy of a gene editing reagent according to any of the methods of Clauses 75 to 106.

134. The kit according to any of Clauses 111 to 133, further comprising a gene editing reagent.

135. The kit according to Clause 134, wherein the gene editing reagent is a reagent of a Clustered Regularly Interspaced Short Palindromic Repeat (CRISPR)/nuclease editing system.

136. The kit according to Clause 134, wherein the gene editing reagent is a reagent of a Transcription Activator-Like Effector Nuclease (TALEN) editing system.

137. The kit according to Clause 134, wherein the gene editing reagent is a reagent of a Zinc-Finger Nuclease (ZFN) editing system.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of identifying whether a cell that has been contacted with a gene editing reagent comprises a desired edit, the method comprising:
a) combining nucleic acids from the cell, or an amplification product thereof, with:
i) a displacer oligonucleotide comprising a 5′ targeting region comprising at least a portion that is complementary to a sequence of the desired edit, a region of the nucleic acid 3′ of the desired edit, or both; and
ii) a flap probe comprising:
a 3′ targeting region comprising at least a portion that is complementary to a sequence of the desired edit, a region of the nucleic acid 5′ of the desired edit, or both; and
a 5′ flap region comprising a hinge nucleotide;
into a reaction mixture under conditions sufficient to produce a junction complex sufficient for cleavage by a flap endonuclease, the junction complex comprising the nucleic acid or amplification product thereof, the displacer oligonucleotide and the flap probe;
b) contacting the reaction mixture with the flap endonuclease under conditions sufficient to release the 5′ flap region from the junction complex; and
c) assaying for the released 5′ flap region, wherein presence of the released 5′ flap region indicates that the cell comprises the desired edit and absence of the released 5′ flap region indicates that the cell does not comprise the desired edit.

2. The method according to claim 1, wherein the cell is a cell of a heterogeneous cell population and step a) comprises combining nucleic acids from the heterogeneous cell population with the displacer oligonucleotide and flap probe.

3. The method according to claim 1, further comprising obtaining the cell from a plurality of cells that have been contacted with the gene editing reagent.

4. The method according to claim 3, wherein obtaining the cell comprises cell sorting.

5. The method according to claim 3 or 4, wherein the cell is a clone produced from the plurality.

6. The method according to claim 5, further comprising producing the clone.

7. The method according to any of the preceding claims, further comprising lysing the cell or heterogeneous cell population prior to the combining.

8. The method according to any of the preceding claims, wherein the combining and the contacting are performed in a reaction vessel of a multi-vessel device.

9. The method according to any of claims 3 to 7, further comprising combining into a plurality of reaction mixtures, each in a separate reaction vessel:
nucleic acids from a cell of the plurality, or an amplification product thereof;
the displacer oligonucleotide; and
the flap probe.

10. The method according to claim 9, further comprising identifying whether each cell of the plurality comprises the desired edit.

11. The method according to any of the preceding claims, further comprising amplifying the nucleic acids or a portion thereof comprising a target site to which the desired edit is targeted.

12. The method according to claim 11, wherein the nucleic acids or the portion thereof is amplified prior to the combining.

13. The method according to any of the preceding claims, further comprising isolating the nucleic acids prior to the combining.

14. The method according to any of the preceding claims, wherein the nucleic acids are genomic nucleic acids.

15. The method according to any of the preceding claims, wherein the flap endonuclease is a FEN1 flap endonuclease.

16. The method according to any of claims 11 to 15, wherein the target site comprises a disease associated allele.

17. The method according to any of claims 11 to 15, wherein the target site does not comprise a disease associated allele.

18. The method according to any of the preceding claims, wherein the desired edit is selected from the group consisting of: a single nucleotide change, an insertion, a deletion or a combination thereof.

19. The method according to claim 18, wherein the desired edit comprises an insertion of a coding sequence.

20. The method according to claim 19, wherein the coding sequence is a heterologous coding sequence.

21. The method according to any of the preceding claims, further comprising contacting the cell with the gene editing reagent.

22. The method according to any of the preceding claims, wherein the gene editing reagent is a component of a Clustered Regularly Interspaced Short Palindromic Repeat (CRISPR)/nuclease system.

23. The method according to claim 22, wherein the CRISPR/nuclease system comprises a guide RNA (gRNA).

24. The method according to any of claims 1 to 21, wherein the gene editing reagent is a component of a Transcription Activator-Like Effector Nuclease (TALEN) system.

25. The method according to claim 24, wherein the TALEN system comprises a TAL effector DNA-binding domain.

26. The method according to any of claims 1 to 21, wherein the gene editing reagent is a component of a Zinc-Finger Nuclease (ZFN) system.

27. The method according to claim 26, wherein the ZFN system comprises a ZFN.

28. The method according to any of the preceding claims, wherein assaying for the released 5′ flap region comprises assaying for a detectable signal.

29. The method according to claim 28, wherein the detectable signal is a fluorescent signal.

30. The method according to claim 29, wherein the flap probe comprises a quenched fluorophore and the fluorescent signal results from unquenching of the quenched fluorophore.

31. The method according to claim 29, wherein the fluorescent signal is generated when the released 5′ flap region hybridizes with a detection cassette, comprising a quenched fluorophore, to form a junction complex that is cleaved by the flap endonuclease, thereby unquenching the quenched fluorophore.

32. The method according to any of the preceding claims, wherein the reaction mixture is contacted with a second flap probe comprising a 3′ targeting region and a 5′ flap region comprising a hinge nucleotide, the second flap probe configured to:
indicate a lack of the desired edit;
indicate a 3′ or 5′ junction of the desired edit;
indicate an undesired edit at the target site to which the desired edit is targeted; or
indicate an edit at a different target site from which the desired edit is targeted.

33. The method according to claim 32, wherein the reaction mixture is contacted with a second displacement oligonucleotide.

34. The method according to claim 33, wherein the second displacement oligonucleotide is configured to form a junction complex with the second flap probe at a different target site from which the desired edit is targeted.

35. The method according to any of claims 32 to 34, wherein the first flap probe and the second flap probe collectively indicate the 3′ and the 5′ junctions of the desired edit.

36. The method according to claim 32, wherein the second flap probe indicates a lack of the desired edit by forming a complex insufficient for cleavage by the flap endonuclease when hybridized to the target site.

37. The method according to any of claims 32 to 34, wherein the 5′ flap region of the second flap probe has a sequence that is configured to not hybridize, when released, with a detection cassette present in the reaction mixture.

38. The method according to any of claims 32 to 35, wherein the 5′ flap region of the second flap probe has a sequence that is configured to hybridize to a detection cassette, comprising a quenched fluorophore, to form a junction complex that is cleaved by the flap endonuclease, thereby unquenching the quenched fluorophore.

39. The method according to any of the preceding claims, wherein the reaction mixture comprises:
a first detection cassette that hybridizes with the 5′ flap region that, when released, indicates that the cell comprises the desired edit; and
a second detection cassette that hybridizes with a 5′ flap region of a second flap probe that, when released, indicates that the cell comprises:
a lack of the desired edit;
a 3′ or 5′ junction of the desired edit;
an undesired edit at the target site to which the desired edit is targeted; or
an edit at a different target site from which the desired edit is targeted.

40. The method according to claim 39, wherein the first detection cassette, prior to hybridizing with the released 5′ flap probe, comprises a quenched fluorophore of a first wavelength and the second detection cassette, prior to hybridizing with the released 5′ flap probe, comprises a quenched fluorophore of a second wavelength.

41. The method according to claim 40, wherein detection of a fluorescent signal at the first wavelength indicates that the cell comprises the desired edit and detection of a fluorescent signal at the second wavelength indicates:
the lack of the desired edit;
presence of the 3′ or 5′ junction of the desired edit;
presence of the undesired edit at the target site; or
presence of the edit at the different target site.

42. The method according to claim 41, wherein detection of the fluorescent signal at the first wavelength and the fluorescent signal at the second wavelength indicates that the cell comprises both the 3′ and the 5′ junction of the desired edit.

43. The method according to any of the preceding claims, wherein the flap probe comprises an extension preventing moiety.

44. The method according to any of the preceding claims, wherein the method further comprises maintaining a separate clone of the cell.

45. The method according to any of the preceding claims, wherein the method further comprises repeating steps (a) through (c) to identify whether the cell has been edited to comprise a second desired edit at a second site different from the first desired edit.

46. The method according to claim 45, wherein repeating the method comprises a displacer oligonucleotide and a flap probe each specific to the second site and configured for determining whether the cell comprises the second desired edit at the second site by assaying for a released 5′ flap region of the flap probe specific for the second site.

47. A method of selecting an edited clone, the method comprising:
a) contacting a plurality of cells with a gene editing reagent;
b) generating a plurality of clones of individual cells of step (a); and
c) identifying whether a clone of step (b) comprises a desired edit according to the method of any of claims 1 to 46 to select an edited clone.

48. The method according to claim 47, wherein the method further comprises assaying a phenotype of the selected edited clone.

49. The method according to claim 47, wherein the method further comprises assaying a phenotype of a cell of the plurality prior to step a) or step b).

50. The method according to claim 48, wherein the assayed phenotype is compared to a corresponding phenotype in a corresponding unedited cell.

51. The method according to any of claims 48 to 50, wherein the phenotype comprises a response to a stimulus.

52. The method according to claim 51, wherein the stimulus is a pharmacological agent.

53. The method according to any of claims 47 to 52, wherein the plurality of cells is a plurality of prokaryotic cells.

54. The method according to claim 53, wherein the plurality of prokaryotic cells comprise bacteria.

55. The method according to any of claims 47 to 52, wherein the plurality of cells is a plurality of eukaryotic cells.

56. The method according to claim 55, wherein the plurality of eukaryotic cells comprise a culture of unicellular eukaryotes.

57. The method according to claim 56, wherein the culture of unicellular eukaryotes comprises unicellular fungi or unicellular algae.

58. The method according to claim 55, wherein the plurality of eukaryotic cells is a primary cell culture derived from a multicellular organism.

59. The method according to claim 55, wherein the plurality of eukaryotic cells is an immortalized cell line derived from a multicellular organism.

60. The method according to claim 55, wherein the method comprises collecting the plurality of eukaryotic cells from a multicellular organism that was administered the gene editing reagent.

61. The method according to any of claims 58 to 60, further comprising incorporating the selected edited clone into a multicellular host.

62. The method according to claim 61, wherein the multicellular host is an embryo.

63. The method according to any of claims 57 to 62, wherein the multicellular organism is selected from the group consisting of: an invertebrate, a vertebrate, a plant, and a fungus.

64. A method of determining whether a polyploid cell, that has been contacted with a gene editing reagent, contains one or more desired edits, the method comprising:
a) combining nucleic acids from the cell, or an amplification product thereof, with:
i) one or more displacer oligonucleotides;
ii) a first flap probe comprising a first 5′ flap region;
iii) a second flap probe comprising a second 5′ flap region;
into a reaction mixture under conditions sufficient to produce first and second junction complexes sufficient for cleavage by a flap endonuclease each complex comprising a displacer oligonucleotide and either the first flap probe or the second flap probe;
b) contacting the reaction mixture with the flap endonuclease under conditions sufficient to release the first and second 5′ flap regions from the first and second junction complexes; and
c) assaying for the first and second 5′ flap regions

65. The method according to claim 64, wherein the first and second junction complexes comprise different displacer oligonucleotides.

66. The method according to claim 64 or 65, wherein assaying for the first and second 5′ flap regions indicate that the polyploid cell contains no desired edits, one desired edit and at a polyploid locus, or two desired edits at the polyploid locus.

67. The method according to claim 64 or 65, wherein assaying for the first and second 5′ flap regions indicates that the polyploid cell contains no desired edits, one desired edit at a first locus, or two desired edits at two different loci.

68. The method according to claim 64 or 65, wherein assaying for the first and second 5′ flap regions indicates that the polyploid cell contains a 3′ junction of a desired edit, a 5′ junction of the desired edit, or both.

69. The method according to any of claims 64 to 68, wherein the first flap probe when cleaved by the flap endonuclease produces a first detectable signal and the second flap probe when cleaved by the flap endonuclease produces a second detectable signal.

70. The method according to claim 69, wherein the first flap probe comprises a first fluorophore and the second flap probe comprises a second fluorophore.

71. The method according to any of claims 64 to 70, wherein the contacting further comprises contacting the reaction mixture with a first detection cassette specific for the released first 5′ flap region and configured to produce a first detectable signal and a second detection cassette specific for the released second 5′ flap region and configured to produce a second detectable signal.

72. The method according to any of claims 69 to 71, wherein the first detectable signal and the second detectable signal are fluorescent signals of different wavelengths.

73. The method according to any of claims 64 to 72, wherein the edit is a single nucleotide change, a deletion, or an insertion.

74. The method according to any of claims 64 to 73, wherein the gene editing reagent is a component of a Clustered Regularly Interspaced Short Palindromic Repeat (CRISPR)/nuclease system.

75. The method according to claim 74, wherein the CRISPR/nuclease system comprises a guide RNA (gRNA).

76. The method according to any of claims 64 to 73, wherein the gene editing reagent is a component of a Transcription Activator-Like Effector Nuclease (TALEN) system.

77. The method according to claim 76, wherein the TALEN system comprises a TAL effector DNA-binding domain.

78. The method according to any of claims 64 to 73, wherein the gene editing reagent is a component of a Zinc-Finger Nuclease (ZFN) system.

79. The method according to claim 78, wherein the ZFN system comprises a ZFN.

80. The method according to any of claims 64 to 79, wherein the polyploid cell is a cell of a heterogeneous population and step a) comprises combining nucleic acids from the heterogeneous population with the one or more displacer oligonucleotides and the flap probes.

81. The method according to any of claims 64 to 80, wherein the method further comprises maintaining a separate clone of the polyploid cell.

82. The method according to any of claims 64 to 81, wherein the polyploid cell is a diploid cell.

83. The method according to claim 82, wherein the diploid cell is a mammalian cell.

84. A method of assessing the efficacy of a gene editing reagent, the method comprising:
a) combining a nucleic acid that has been contacted with the gene editing reagent that is configured to produce a desired edit with:
i) a displacer oligonucleotide comprising a 5′ targeting region; and
ii) a flap probe comprising a 3′ targeting region and a 5′ flap region comprising a hinge nucleotide;
into a reaction mixture under conditions sufficient to produce a junction complex sufficient for cleavage by a flap endonuclease, the junction complex comprising the nucleic acid, the displacer oligonucleotide and the flap probe;
b) contacting the reaction mixture with the flap endonuclease under conditions sufficient to release the 5′ flap region from the junction complex; and
c) assaying for the released 5′ flap region to assess the efficacy of the gene editing reagent.

85. The method according to claim 84, further comprising amplifying the nucleic acid or a portion thereof comprising a target site to which the desired edit is targeted.

86. The method according to claim 85, wherein the nucleic acid or the portion thereof is amplified prior to the combining.

87. The method according to any of claims 84 to 86, further comprising isolating the nucleic acid prior to the combining.

88. The method according to any of claims 84 to 87, wherein the flap endonuclease is a FEN1 flap endonuclease.

89. The method according to any of claims 84 to 88, wherein the target site to which the desired edit is directed comprises a disease associated allele.

90. The method according to any of claims 84 to 89, wherein the desired edit comprises a single nucleotide substitution, a deletion or an insertion.

91. The method according to any of claims 84 to 90, further comprising contacting the nucleic acid with the gene editing reagent.

92. The method according to any of claims 84 to 91, wherein the gene editing reagent is a component of a Clustered Regularly Interspaced Short Palindromic Repeat (CRISPR)/nuclease system.

93. The method according to claim 92, wherein the CRISPR/nuclease system comprises a guide RNA (gRNA).

94. The method according to any of claims 84 to 91, wherein the gene editing reagent is a component of a Transcription Activator-Like Effector Nuclease (TALEN) system.

95. The method according to claim 94, wherein the TALEN system comprises a TAL effector DNA-binding domain.

96. The method according to any of claims 84 to 91, wherein the gene editing reagent is a component of a Zinc-Finger Nuclease (ZFN) system.

97. The method according to claim 96, wherein the ZFN system comprises a ZFN.

98. The method according to any of claims 84 to 97, wherein assaying for the released 5′ flap region comprises assaying for a fluorescent signal.

99. The method according to claim 98, wherein the flap probe comprises a quenched fluorophore and the fluorescent signal results from unquenching of the quenched fluorophore.

100. The method according to claim 98, wherein the fluorescent signal is generated when the released 5′ flap region hybridizes with a detection cassette, comprising a quenched fluorophore, to form a junction complex that is cleaved by the flap endonuclease, thereby unquenching the quenched fluorophore.

101. The method according to claim 100, comprising contacting the reaction mixture with the detection cassette.

102. The method according to any of claims 84 to 101, wherein the reaction mixture further comprises a second flap probe comprising a 3′ targeting region and a 5′ flap region comprising a hinge nucleotide, the second flap probe configured to:
indicate a lack of the desired edit;
indicate a 3′ or 5′ junction of the desired edit;
indicate an undesired edit at the target site to which the desired edit is targeted; or
indicate an edit at a different target site from which the desired edit is targeted.

103. The method according to claim 102, wherein the reaction mixture is contacted with a second displacement oligonucleotide.

104. The method according to claim 103, wherein the second displacement oligonucleotide is configured to form a junction complex with the second flap probe at a different target site from which the desired edit is targeted.

105. The method according to any of claims 102 to 104, wherein the first flap probe and the second flap probe collectively indicate the 3′ and the 5′ junctions of the desired edit.

106. The method according to claim 102, wherein the second flap probe indicates a lack of the desired edit by forming a complex insufficient for cleavage by the flap endonuclease when hybridized to the target site.

107. The method according to any of claims 102 to 106, wherein the 5′ flap region of the second flap probe has a sequence that is configured to not hybridize, when released, with a detection cassette present in the reaction mixture.

108. The method according to any of claims 102 to 107, wherein the 5′ flap region of the second flap probe has a sequence that is configured to hybridize, when released, to a second detection cassette to generate a second fluorescent signal.

109. The method according to any of claims 102 to 108, wherein the method comprises comparing detection of the 5′ flap region released from the first flap probe with detection of the 5′ flap region released from the second flap probe.

110. The method according to any of claims 84 to 109, wherein the flap probe comprises an extension preventing moiety.

111. The method according to claim 110, wherein the extension preventing moiety comprises a 3′ hexanediol.

112. The method according to any of claims 84 to 111, wherein the method assesses the efficacy of a plurality of gene editing reagents each combined into an individual reaction mixture present in an individual reaction vessel.

113. The method according to claim 112, wherein the plurality of gene editing reagents all target the same target site.

114. The method according to claim 112 or 113, wherein the plurality of gene editing reagents are all configured to introduce the same edit.

115. The method according to any of claims 112 to 114, wherein the plurality of gene editing reagents comprise a plurality of different gRNAs.

116. The method according to any of claims 112 to 114, wherein the plurality of gene editing reagents comprise a plurality of different TAL effector DNA-binding domains.

117. The method according to any of claims 112 to 114, wherein the plurality of gene editing reagents comprise a plurality of different ZFNs.

118. The method according to any of claims 84 to 117, wherein the method is a non-diagnostic method.

119. A method of editing a nucleic acid, the method comprising:
a) assessing the efficacy of a plurality of gene editing reagents using the method according to any of claims 84 to 118;
b) selecting a gene editing reagent based on the assessed efficacy; and
c) editing the nucleic acid using the selected gene editing reagent.

120. The method according to claim 119, wherein the nucleic acid is genomically integrated.

121. The method according to claim 120, wherein the nucleic acid is integrated in a prokaryotic genome.

122. The method according to claim 120, wherein the nucleic acid is integrated in a eukaryotic genome.

123. A kit comprising:
a flap endonuclease;
a detection cassette; and
a set of positive control nucleic acids that confirm the presence of a desired edit of a control target nucleic acid.

124. The kit according to claim 123, wherein the set of positive control nucleic acids comprises a displacer oligonucleotide, a flap probe and the control target nucleic acid comprising the desired edit.

125. The kit according to claim 123 or 124, wherein the set of positive control nucleic acids are present in a single container.

126. The kit according to any of claims 123 to 125, further comprising a set of negative control nucleic acids that confirm the absence of the desired edit.

127. The kit according to claim 126, wherein the set of negative control nucleic acids comprises a displacer oligonucleotide, a flap probe and a negative control nucleic acid comprising an unedited or misedited target site.

128. The kit according to claim 127, wherein the misedited target site comprises a truncated insertion.

129. The kit according to claim 126 or 128, wherein the set of negative control nucleic acids are present in a single container.

130. The kit according to any of claims 123 to 129, wherein the flap endonuclease is a FEN1 flap endonuclease.

131. The kit according to any of claims 123 to 130, wherein the detection cassette comprises a fluorophore and a quencher.

132. The kit according to any of claims 123 to 131, wherein the flap probe comprises an extension preventing moiety.

133. The kit according to claim 132, wherein the extension preventing moiety comprises a 3′ hexanediol.

134. The kit according to any of claims 123 to 133, further comprising an annealing buffer.

135. The kit according to any of claims 123 to 134, further comprising an endonuclease buffer.

136. The kit according to any of claims 123 to 135, further comprising a nucleic acid dilution buffer.

137. The kit according to any of claims 123 to 136, further comprising an amplification polymerase.

138. The kit according to any of claims 123 to 137, further comprising an amplification polymerase buffer.

139. The kit according to any of claims 123 to 138, further comprising a nucleic acid extraction reagent.

140. The kit according to any of claims 123 to 139, further comprising a cell lysis reagent.

141. The kit according to claim 140, wherein the nucleic acid extraction reagent and the cell lysis reagent are in the same container.

142. The kit according to any of claims 123 to 141, wherein the kit further comprises two or more different flap probes and/or two or more different displacement oligonucleotides for the set of positive control nucleic acids.

143. The kit according to any of claims 123 to 142, further comprising a second detection cassette.

144. The kit according to any of claims 123 to 143, further comprising instructions for identifying whether a cell that has been contacted with a gene editing reagent contains a desired edit according to any of the methods of claims 1 to 46.

145. The kit according to any of claims 123 to 143, further comprising instructions for identifying whether a polypoid cell that has been contacted with a gene editing reagent contains one or more desired edits according to any of the methods of claims 64 to 83.

146. The kit according to any of claims 123 to 143, further comprising instructions for assessing the efficacy of a gene editing reagent according to any of the methods of claims 84 to 118.

147. The kit according to any of claims 123 to 146, further comprising a gene editing reagent.

148. The kit according to claim 147, wherein the gene editing reagent is a reagent of a Clustered Regularly Interspaced Short Palindromic Repeat (CRISPR)/nuclease editing system.

149. The kit according to claim 147, wherein the gene editing reagent is a reagent of a Transcription Activator-Like Effector Nuclease (TALEN) editing system.

150. The kit according to claim 147, wherein the gene editing reagent is a reagent of a Zinc-Finger Nuclease (ZFN) editing system.
